(19) United States
(12) Patent Application Publication
(10) Pub. No.: US 2025/0188020 A1
(43) Pub. Date: Jun. 12, 2025

(54) LIPIDS FOR USE IN LIPID NANOPARTICLE FORMULATIONS

(71) Applicant: Acuitas Therapeutics, Inc., Vancouver (CA)

(72) Inventors: Xinyao Du, Richmond (CA); Steven M. Ansell, Vancouver (CA); Jason Samuel Tan, Vancouver (CA); Polina Blagojevic, Vancouver (CA); Stephen Paul Arns, Vancouver (CA); Julia Gatenyo, Vancouver (CA)

(21) Appl. No.: 18/886,871

(22) Filed: Sep. 16, 2024

Related U.S. Application Data
(63) Continuation of application No. 18/066,954, filed on Dec. 15, 2022, now Pat. No. 12,129,223.
(60) Provisional application No. 63/290,398, filed on Dec. 16, 2021.

(51) Int. Cl. C07C 237/06; A61K 9/51; A61K 31/7105; C07C 237/22; C07D 233/64

(52) U.S. Cl. CPC C07C 237/06; A61K 9/5123; A61K 31/7105; C07C 237/22; C07D 233/64; C07C 2601/04

(57) ABSTRACT

Compounds are provided having the following structure:

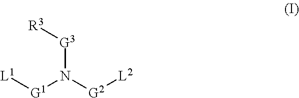

or a pharmaceutically acceptable salt, tautomer or stereoisomer thereof, wherein R³, L¹, L², G¹, G² and G³ are as defined herein. Use of the compounds as a component of lipid nanoparticle formulations for delivery of a therapeutic agent, compositions comprising the compounds and methods for their use and preparation are also provided.

### BACKGROUND

### Technical Field

Embodiments of the present invention generally relate to novel lipids that can be used in combination with other lipid components, such as neutral lipids, cholesterol and polymer conjugated lipids, to form lipid nanoparticles for delivery of therapeutic agents, such as nucleic acids (e.g., oligonucleotides, messenger RNA), both in vitro and in vivo.

### Description of the Related Art

There are many challenges associated with the delivery of nucleic acids to affect a desired response in a biological system. Nucleic acid based therapeutics have enormous potential but there remains a need for more effective delivery of nucleic acids to appropriate sites within a cell or organism in order to realize this potential. Therapeutic nucleic acids include, e.g., messenger RNA (mRNA), antisense oligonucleotides, ribozymes, DNAzymes, plasmids, immune stimulating nucleic acids, antagomir, antimir, mimic, supermir, and aptamers. Some nucleic acids, such as mRNA or plasmids, can be used to effect expression of specific cellular products as would be useful in the treatment of, for example, diseases related to a deficiency of a protein or enzyme. The therapeutic applications of translatable nucleotide delivery are extremely broad as constructs can be synthesized to produce any chosen protein sequence, whether or not indigenous to the system. The expression products of the nucleic acid can augment existing levels of protein, replace missing or non-functional versions of a protein, or introduce new protein and associated functionality in a cell or organism.

Some nucleic acids, such as miRNA inhibitors, can be used to effect expression of specific cellular products that are regulated by miRNA as would be useful in the treatment of, for example, diseases related to deficiency of protein or enzyme. The therapeutic applications of miRNA inhibition are extremely broad as constructs can be synthesized to inhibit one or more miRNA that would in turn regulate the expression of mRNA products. The inhibition of endogenous miRNA can augment its downstream target endogenous protein expression and restore proper function in a cell or organism as a means to treat disease associated to a specific miRNA or a group of miRNA.

Other nucleic acids can down-regulate intracellular levels of specific mRNA and, as a result, down-regulate the synthesis of the corresponding proteins through processes such as RNA interference (RNAi) or complementary binding of antisense RNA. The therapeutic applications of antisense oligonucleotide and RNAi are also extremely broad, since oligonucleotide constructs can be synthesized with any nucleotide sequence directed against a target mRNA. Targets may include mRNAs from normal cells, mRNAs associated with disease-states, such as cancer, and mRNAs of infectious agents, such as viruses. To date, antisense oligonucleotide constructs have shown the ability to specifically down-regulate target proteins through degradation of the cognate mRNA in both in vitro and in vivo models. In addition, antisense oligonucleotide constructs are currently being evaluated in clinical studies.

However, two problems currently face the use of oligonucleotides in therapeutic contexts. First, free RNAs are susceptible to nuclease digestion in plasma. Second, free RNAs have limited ability to gain access to the intracellular compartment where the relevant translation machinery resides. Lipid nanoparticles formed from lipids formulated with other lipid components, such as neutral lipids, cholesterol, PEG, PEGylated lipids, and oligonucleotides have been used to block degradation of the RNAs in plasma and facilitate the cellular uptake of the oligonucleotides.

There remains a need for improved lipids and lipid nanoparticles for the delivery of oligonucleotides. Preferably, these lipid nanoparticles would provide optimal drug:lipid ratios, protect the nucleic acid from degradation and clearance in serum, be suitable for systemic or local delivery, and provide intracellular delivery of the nucleic acid. In addition, these lipid-nucleic acid particles should be well-tolerated and provide an adequate therapeutic index, such that patient treatment at an effective dose of the nucleic acid is not associated with unacceptable toxicity and/or risk to the patient. The present invention provides these and related advantages.

### BRIEF SUMMARY

In brief, embodiments of the present invention provide lipid compounds, including stereoisomers, pharmaceutically acceptable salts, prodrugs or tautomers thereof, which can be used alone or in combination with other lipid components such as neutral lipids, charged lipids, steroids (including for example, all sterols) and/or their analogs, and/or polymer conjugated lipids to form lipid nanoparticles for the delivery of therapeutic agents. In some instances, the lipid nanoparticles are used to deliver nucleic acids such as antisense and/or messenger RNA. Methods for use of such lipid nanoparticles for treatment of various diseases or conditions, such as those caused by infectious entities and/or insufficiency of a protein, are also provided.

In one embodiment, compounds having the following structure (I) are provided:

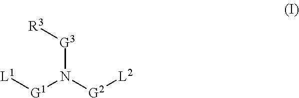

or a pharmaceutically acceptable salt, tautomer, prodrug or stereoisomer thereof, wherein R³, L¹, L², G¹, G², and G³ are as defined herein.

Pharmaceutical compositions comprising one or more of the foregoing compounds of structure (I) and a therapeutic agent are also provided. Also provided are lipid nanoparticles (LNPs) comprising one or more compounds of structure (I). In some embodiments, the pharmaceutical compositions and/or LNPs further comprise one or more components selected from neutral lipids, charged lipids, steroids and polymer conjugated lipids. The disclosed compositions are useful for formation of lipid nanoparticles for the delivery of the therapeutic agent.

In other embodiments, the present invention provides a method for administering a therapeutic agent to a patient in need thereof, the method comprising preparing a composition of lipid nanoparticles comprising the compound of structure (I) and a therapeutic agent and delivering the composition to the patient. In some embodiments the method for administering a therapeutic agent to a patient in need thereof comprises administering an LNP comprising one or more compounds of structure (I) and the therapeutic agent to the patient.

These and other aspects of the invention will be apparent upon reference to the following detailed description.

### DETAILED DESCRIPTION

In the following description, certain specific details are set forth in order to provide a thorough understanding of various embodiments of the invention. However, one skilled in the art will understand that embodiments of the invention may be practiced without these details.

Embodiments of the present invention are based, in part, upon the discovery of novel lipids that provide advantages when used in lipid nanoparticles for the in vivo delivery of an active or therapeutic agent such as a nucleic acid into a cell of a mammal. In particular, embodiments the present invention provides nucleic acid-lipid nanoparticle compositions comprising one or more of the novel lipids described herein that provide increased activity of the nucleic acid and improved tolerability of the compositions in vivo, resulting in a significant increase in the therapeutic index as compared to nucleic acid-lipid nanoparticle compositions previously described. For example, embodiments provide a lipid nanoparticle comprising one or more compounds of structure (I).

In particular embodiments, the present invention provides novel lipids that enable the formulation of improved compositions for the in vitro and in vivo delivery of mRNA and/or other oligonucleotides. In some embodiments, these improved lipid nanoparticle compositions are useful for expression of protein encoded by mRNA. In other embodiments, these improved lipid nanoparticles compositions are useful for upregulation of endogenous protein expression by delivering miRNA inhibitors targeting one specific miRNA or a group of miRNA regulating one target mRNA or several mRNA. In other embodiments, these improved lipid nanoparticle compositions are useful for down-regulating (e.g., silencing) the protein levels and/or mRNA levels of target genes. In some other embodiments, the lipid nanoparticles are also useful for delivery of mRNA and plasmids for expression of transgenes. In yet other embodiments, the lipid nanoparticle compositions are useful for inducing a pharmacological effect resulting from expression of a protein, e.g., increased production of red blood cells through the delivery of a suitable erythropoietin mRNA, or protection against infection through delivery of mRNA encoding for a suitable antigen or antibody.

The lipid nanoparticles and compositions of embodiments of the present invention may be used for a variety of purposes, including the delivery of encapsulated or associated (e.g., complexed) therapeutic agents such as nucleic acids to cells, both in vitro and in vivo. Accordingly, embodiments of the present invention provide methods of treating or preventing diseases or disorders in a subject in need thereof by contacting the subject with a lipid nanoparticle that encapsulates or is associated with a suitable therapeutic agent, wherein the lipid nanoparticle comprises one or more of the novel lipids described herein.

As described herein, embodiments of the lipid nanoparticles of the present invention are particularly useful for the delivery of nucleic acids, including, e.g., mRNA, antisense oligonucleotide, plasmid DNA, microRNA (miRNA), miRNA inhibitors (antagomirs/antimirs), messenger-RNA-interfering complementary RNA (micRNA), DNA, multivalent RNA, dicer substrate RNA, complementary DNA (cDNA), etc. Therefore, the lipid nanoparticles and compositions of embodiments of the present invention may be used to induce expression of a desired protein both in vitro and in vivo by contacting cells with a lipid nanoparticle comprising one or more novel lipids described herein, wherein the lipid nanoparticle encapsulates or is associated with a nucleic acid that is expressed to produce the desired protein (e.g., a messenger RNA or plasmid encoding the desired protein) or inhibit processes that terminate expression of mRNA (e.g., miRNA inhibitors). Alternatively, the lipid nanoparticles and compositions of embodiments of the present invention may be used to decrease the expression of target genes and proteins both in vitro and in vivo by contacting cells with a lipid nanoparticle comprising one or more novel lipids (e.g., a compound of structure (I)) described herein, wherein the lipid nanoparticle encapsulates or is associated with a nucleic acid that reduces target gene expression (e.g., an antisense oligonucleotide or small interfering RNA (siRNA)). The lipid nanoparticles and compositions of embodiments of the present invention may also be used for co-delivery of different nucleic acids (e.g., mRNA and plasmid DNA) separately or in combination, such as may be useful to provide an effect requiring colocalization of different nucleic acids (e.g., mRNA encoding for a suitable gene modifying enzyme and DNA segment(s) for incorporation into the host genome).

Nucleic acids for use with embodiments of this invention may be prepared according to any available technique. For mRNA, the primary methodology of preparation is, but not limited to, enzymatic synthesis (also termed in vitro transcription) which currently represents the most efficient method to produce long sequence-specific mRNA. In vitro transcription describes a process of template-directed synthesis of RNA molecules from an engineered DNA template comprised of an upstream bacteriophage promoter sequence (e.g., including but not limited to that from the T7, T3 and SP6 coliphage) linked to a downstream sequence encoding the gene of interest. Template DNA can be prepared for in vitro transcription from a number of sources with appropriate techniques which are well known in the art including, but not limited to, plasmid DNA and polymerase chain reaction amplification (see Linpinsel, J. L and Conn, G. L., General protocols for preparation of plasmid DNA template and Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012)

Transcription of the RNA occurs in vitro using the linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art. (see, e.g., Losick, R., 1972, In vitro transcription, Ann Rev Biochem v.41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed mRNA is then purified from the undesired components of the transcription or associated reactions (including unincorporated rNTPs, protein enzyme, salts, short RNA oligos, etc.). Techniques for the isolation of the mRNA transcripts are well known in the art. Well known procedures include phenol/chloroform extraction or precipitation with either alcohol (ethanol, isopropanol) in the presence of monovalent cations or lithium chloride. Additional, non-limiting examples of purification procedures which can be used include size exclusion chromatography (Lukavsky, P. J. and Puglisi, J. D., 2004, Large-scale preparation and purification of polyacrylamide-free RNA oligonucleotides, RNA v.10, 889-893), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012). Purification can be performed using a variety of commercially available kits including, but not limited to SV Total Isolation System (Promega) and In Vitro Transcription Cleanup and Concentration Kit (Norgen Biotek).

Furthermore, while reverse transcription can yield large quantities of mRNA, the products can contain a number of aberrant RNA impurities associated with undesired polymerase activity which may need to be removed from the full-length mRNA preparation. These include short RNAs that result from abortive transcription initiation as well as double-stranded RNA (dsRNA) generated by RNA-dependent RNA polymerase activity, RNA-primed transcription from RNA templates and self-complementary 3′ extension. It has been demonstrated that these contaminants with dsRNA structures can lead to undesired immunostimulatory activity through interaction with various innate immune sensors in eukaryotic cells that function to recognize specific nucleic acid structures and induce potent immune responses. This in turn, can dramatically reduce mRNA translation since protein synthesis is reduced during the innate cellular immune response. Therefore, additional techniques to remove these dsRNA contaminants have been developed and are known in the art including but not limited to scaleable HPLC purification (see, e.g., Kariko, K., Muramatsu, H., Ludwig, J. And Weissman, D., 2011, Generating the optimal mRNA for therapy: HPLC purification eliminates immune activation and improves translation of nucleoside-modified, protein-encoding mRNA, Nucl Acid Res, v. 39 e142; Weissman, D., Pardi, N., Muramatsu, H., and Kariko, K., HPLC Purification of in vitro transcribed long RNA in Synthetic Messenger RNA and Cell Metabolism Modulation in Methods in Molecular Biology v.969 (Rabinovich, P. H. Ed), 2013). HPLC purified mRNA has been reported to be translated at much greater levels, particularly in primary cells and in vivo.

A significant variety of modifications have been described in the art which are used to alter specific properties of in vitro transcribed mRNA, and improve its utility. These include, but are not limited to modifications to the 5′ and 3′ termini of the mRNA. Endogenous eukaryotic mRNA typically contain a cap structure on the 5′-end of a mature molecule which plays an important role in mediating binding of the mRNA Cap Binding Protein (CBP), which is in turn responsible for enhancing mRNA stability in the cell and efficiency of mRNA translation. Therefore, highest levels of protein expression are achieved with capped mRNA transcripts. The 5′-cap contains a 5′-5′-triphosphate linkage between the 5′-most nucleotide and guanine nucleotide. The conjugated guanine nucleotide is methylated at the N7 position. Additional modifications include methylation of the ultimate and penultimate most 5′-nucleotides on the 2′-hydroxyl group.

Multiple distinct cap structures can be used to generate the 5′-cap of in vitro transcribed synthetic mRNA. 5′-capping of synthetic mRNA can be performed co-transcriptionally with chemical cap analogs (i.e. capping during in vitro transcription). For example, the Anti-Reverse Cap Analog (ARCA) cap contains a 5′-5′-triphosphate guanine-guanine linkage where one guanine contains an N7 methyl group as well as a 3′-O-methyl group. However, up to 20% of transcripts remain uncapped during this co-transcriptional process and the synthetic cap analog is not identical to the 5′-cap structure of an authentic cellular mRNA, potentially reducing translatability and cellular stability. Alternatively, synthetic mRNA molecules may also be enzymatically capped post-transcriptionally. These may generate a more authentic 5′-cap structure that more closely mimics, either structurally or functionally, the endogenous 5′-cap which have enhanced binding of cap binding proteins, increased half-life, reduced susceptibility to 5′ endonucleases, and/or reduced 5′ decapping. Numerous synthetic 5′-cap analogs have been developed and are known in the art to enhance mRNA stability and translatability (see, e.g., Grudzien-Nogalska, E., Kowalska, J., Su, W., Kuhn, A. N., Slepenkov, S. V., Darynkiewicz, E., Sahin, U., Jemielity, J., and Rhoads, R. E., Synthetic mRNAs with superior translation and stability properties in Synthetic Messenger RNA and Cell Metabolism Modulation in Methods in Molecular Biology v.969 (Rabinovich, P. H. Ed), 2013).

On the 3′-terminus, a long chain of adenine nucleotides (poly-A tail) is normally added to mRNA molecules during RNA processing. Immediately after transcription, the 3′ end of the transcript is cleaved to free a 3′ hydroxyl to which poly-A polymerase adds a chain of adenine nucleotides to the RNA in a process called polyadenylation. The poly-A tail has been extensively shown to enhance both translational efficiency and stability of mRNA (see Bernstein, P. and Ross, J., 1989, Poly (A), poly (A) binding protein and the regulation of mRNA stability, Trends Bio Sci v. 14 373-377; Guhaniyogi, J. And Brewer, G., 2001, Regulation of mRNA stability in mammalian cells, Gene, v. 265, 11-23; Dreyfus, M. And Regnier, P., 2002, The poly (A) tail of mRNAs: Bodyguard in eukaryotes, scavenger in bacteria, Cell, v.111, 611-613).

Poly (A) tailing of in vitro transcribed mRNA can be achieved using various approaches including, but not limited to, cloning of a poly (T) tract into the DNA template or by post-transcriptional addition using Poly (A) polymerase. The first case allows in vitro transcription of mRNA with poly (A) tails of defined length, depending on the size of the poly (T) tract, but requires additional manipulation of the template. The latter case involves the enzymatic addition of a poly (A) tail to in vitro transcribed mRNA using poly (A) polymerase which catalyzes the incorporation of adenine residues onto the 3′termini of RNA, requiring no additional manipulation of the DNA template, but results in mRNA with poly(A) tails of heterogeneous length. 5′-capping and 3′-poly (A) tailing can be performed using a variety of commercially available kits including, but not limited to Poly (A) Polymerase Tailing kit (EpiCenter), mMESSAGE mMACHINE T7 Ultra kit and Poly (A) Tailing kit (Life Technologies) as well as with commercially available reagents, various ARCA caps, Poly (A) polymerase, etc.

In addition to 5′ cap and 3′ poly adenylation, other modifications of the in vitro transcripts have been reported to provide benefits as related to efficiency of translation and stability. It is well known in the art that pathogenic DNA and RNA can be recognized by a variety of sensors within eukaryotes and trigger potent innate immune responses. The ability to discriminate between pathogenic and self DNA and RNA has been shown to be based, at least in part, on structure and nucleoside modifications since most nucleic acids from natural sources contain modified nucleosides In contrast, in vitro synthesized RNA lacks these modifications, thus rendering it immunostimulatory which in turn can inhibit effective mRNA translation as outlined above. The introduction of modified nucleosides into in vitro transcribed mRNA can be used to prevent recognition and activation of RNA sensors, thus mitigating this undesired immunostimulatory activity and enhancing translation capacity (see e.g. Kariko, K. And Weissman, D. 2007, Naturally occurring nucleoside modifications suppress the immunostimulatory activity of RNA: implication for therapeutic RNA development, Curr Opin Drug Discov Devel, v.10 523-532; Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v.969 (Rabinovich, P. H. Ed), 2013); Kariko, K., Muramatsu, H., Welsh, F. A., Ludwig, J., Kato, H., Akira, S., Weissman, D., 2008, Incorporation of Pseudouridine Into mRNA Yields Superior Nonimmunogenic Vector With Increased Translational Capacity and Biological Stability, Mol Ther v.16, 1833-1840. The modified nucleosides and nucleotides used in the synthesis of modified RNAs can be prepared monitored and utilized using general methods and procedures known in the art. A large variety of nucleoside modifications are available that may be incorporated alone or in combination with other modified nucleosides to some extent into the in vitro transcribed mRNA (see e.g.US2012/0251618). In vitro synthesis of nucleoside-modified mRNA have been reported to have reduced ability to activate immune sensors with a concomitant enhanced translational capacity.

Other components of mRNA which can be modified to provide benefit in terms of translatability and stability include the 5′ and 3′ untranslated regions (UTR). Optimization of the UTRs (favorable 5′ and 3′ UTRs can be obtained from cellular or viral RNAs), either both or independently, have been shown to increase mRNA stability and translational efficiency of in vitro transcribed mRNA (see e.g. Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v.969 (Rabinovich, P. H. Ed), 2013).

In addition to mRNA, other nucleic acid payloads may be used for embodiments of this invention. For oligonucleotides, methods of preparation include but are not limited to chemical synthesis and enzymatic, chemical cleavage of a longer precursor, in vitro transcription as described above, etc. Methods of synthesizing DNA and RNA nucleotides are widely used and well known in the art (see, e.g. Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

For plasmid DNA, preparation for use with embodiments of this invention commonly utilizes but is not limited to expansion and isolation of the plasmid DNA in vitro in a liquid culture of bacteria containing the plasmid of interest. The presence of a gene in the plasmid of interest that encodes resistance to a particular antibiotic (penicillin, kanamycin, etc.) allows those bacteria containing the plasmid of interest to selectively grow in antibiotic-containing cultures. Methods of isolating plasmid DNA are widely used and well known in the art (see, e.g. Heilig, J., Elbing, K. L. and Brent, R (2001) Large-Scale Preparation of Plasmid DNA. Current Protocols in Molecular Biology. 41:11:1.7:1.7.1-1.7.16; Rozkov, A., Larsson, B., Gillström, S., Björnestedt, R. and Schmidt, S. R. (2008), Large-scale production of endotoxin-free plasmids for transient expression in mammalian cell culture. Biotechnol. Bioeng., 99: 557-566; and U.S. Pat. No. 6,197,553B1). Plasmid isolation can be performed using a variety of commercially available kits including, but not limited to Plasmid Plus (Qiagen), GenJET plasmid MaxiPrep (Thermo) and PureYield MaxiPrep (Promega) kits as well as with commercially available reagents.

Various exemplary embodiments of the lipids of the present invention, lipid nanoparticles and compositions comprising the same, and their use to deliver active (e.g. therapeutic agents), such as nucleic acids, to modulate gene and protein expression, are described in further detail below.

As used herein, the following terms have the meanings ascribed to them unless specified otherwise.

Unless the context requires otherwise, throughout the present specification and claims, the word “comprise” and variations thereof, such as, “comprises” and “comprising” are to be construed in an open and inclusive sense, that is, as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless defined otherwise, all technical and scientific terms used herein have the same meaning as is commonly understood by one of skill in the art to which this invention belongs. As used in the specification and claims, the singular form “a”, “an” and “the” include plural references unless the context clearly dictates otherwise.

The phrase “induce expression of a desired protein” refers to the ability of a nucleic acid to increase expression of the desired protein. To examine the extent of protein expression, a test sample (e.g. a sample of cells in culture expressing the desired protein) or a test mammal (e.g. a mammal such as a human or an animal model such as a rodent (e.g. mouse) or a non-human primate (e.g., monkey) model) is contacted with a nucleic acid (e.g. nucleic acid in combination with a lipid of the present invention). Expression of the desired protein in the test sample or test animal is compared to expression of the desired protein in a control sample (e.g. a sample of cells in culture expressing the desired protein) or a control mammal (e.g., a mammal such as a human or an animal model such as a rodent (e.g. mouse) or non-human primate (e.g. monkey) model) that is not contacted with or administered the nucleic acid. When the desired protein is present in a control sample or a control mammal, the expression of a desired protein in a control sample or a control mammal may be assigned a value of 1.0. In particular embodiments, inducing expression of a desired protein is achieved when the ratio of desired protein expression in the test sample or the test mammal to the level of desired protein expression in the control sample or the control mammal is greater than 1, for example, about 1.1, 1.5, 2.0. 5.0 or 10.0. When a desired protein is not present in a control sample or a control mammal, inducing expression of a desired protein is achieved when any measurable level of the desired protein in the test sample or the test mammal is detected. One of ordinary skill in the art will understand appropriate assays to determine the level of protein expression in a sample, for example dot blots, northern blots, in situ hybridization, ELISA, immunoprecipitation, enzyme function, and phenotypic assays, or assays based on reporter proteins that can produce fluorescence or luminescence under appropriate conditions.

The phrase “inhibiting expression of a target gene” refers to the ability of a nucleic acid to silence, reduce, or inhibit the expression of a target gene. To examine the extent of gene silencing, a test sample (e.g. a sample of cells in culture expressing the target gene) or a test mammal (e.g. a mammal such as a human or an animal model such as a rodent (e.g. mouse) or a non-human primate (e.g. monkey) model) is contacted with a nucleic acid that silences, reduces, or inhibits expression of the target gene. Expression of the target gene in the test sample or test animal is compared to expression of the target gene in a control sample (e.g. a sample of cells in culture expressing the target gene) or a control mammal (e.g. a mammal such as a human or an animal model such as a rodent (e.g. mouse) or non-human primate (e.g. monkey) model) that is not contacted with or administered the nucleic acid. The expression of the target gene in a control sample or a control mammal may be assigned a value of 100%. In particular embodiments, silencing, inhibition, or reduction of expression of a target gene is achieved when the level of target gene expression in the test sample or the test mammal relative to the level of target gene expression in the control sample or the control mammal is about 95%, 90%, 85%, 80%, 75%, 70%, 65%, 60%, 55%, 50%, 45%, 40%, 35%, 30%, 25%, 20%, 15%, 10%, 5%, or 0%. In other words, the nucleic acids are capable of silencing, reducing, or inhibiting the expression of a target gene by at least about 5%, 10%, 15%, 20%, 25%, 30%, 35%, 40%, 45%, 50%, 55%, 60%, 65%, 70%, 75%, 80%, 85%, 90%, 95%, or 100% in a test sample or a test mammal relative to the level of target gene expression in a control sample or a control mammal not contacted with or administered the nucleic acid. Suitable assays for determining the level of target gene expression include, without limitation, examination of protein or mRNA levels using techniques known to those of skill in the art, such as, e.g., dot blots, northern blots, in situ hybridization, ELISA, immunoprecipitation, enzyme function, as well as phenotypic assays known to those of skill in the art.

An “effective amount” or “therapeutically effective amount” of an active agent or therapeutic agent such as a therapeutic nucleic acid is an amount sufficient to produce the desired effect, e.g. an increase or inhibition of expression of a target sequence in comparison to the normal expression level detected in the absence of the nucleic acid. An increase in expression of a target sequence is achieved when any measurable level is detected in the case of an expression product that is not present in the absence of the nucleic acid. In the case where the expression product is present at some level prior to contact with the nucleic acid, an in increase in expression is achieved when the fold increase in value obtained with a nucleic acid such as mRNA relative to control is about 1.05, 1.1, 1.2, 1.3, 1.4, 1.5, 1.75, 2, 2.5, 3, 4, 5, 6, 7, 8, 9, 10, 15, 20, 25, 30, 40, 50, 75, 100, 250, 500, 750, 1000, 5000, 10000 or greater. Inhibition of expression of a target gene or target sequence is achieved when the value obtained with a nucleic acid such as antisense oligonucleotide relative to the control is about 95%, 90%, 85%, 80%, 75%, 70%, 65%, 60%, 55%, 50%, 45%, 40%, 35%, 30%, 25%, 20%, 15%, 10%, 5%, or 0%. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, fluorescence or luminescence of suitable reporter proteins, as well as phenotypic assays known to those of skill in the art.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The term “lipid” refers to a group of organic compounds that include, but are not limited to, esters of fatty acids and are generally characterized by being poorly soluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

A “steroid” is a compound comprising the following carbon skeleton:

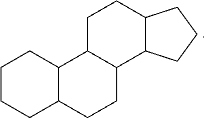 [SMILES: C1CCC2C(C1)CCC1C3CCCC3CCC21]

Non-limiting examples of steroids include cholesterol, and the like.

A “cationic lipid” refers to a lipid capable of being positively charged. Exemplary cationic lipids include one or more amine group(s) which bear the positive charge. Preferred cationic lipids are ionizable such that they can exist in a positively charged or neutral form depending on pH. The ionization of the cationic lipid affects the surface charge of the lipid nanoparticle under different pH conditions. This charge state can influence plasma protein absorption, blood clearance and tissue distribution (Semple, S. C., et al., Adv. Drug Deliv Rev 32:3-17 (1998)) as well as the ability to form endosomolytic non-bilayer structures (Hafez, I. M., et al., Gene Ther 8:1188-1196 (2001)) critical to the intracellular delivery of nucleic acids.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-DMG) and the like.

The term “neutral lipid” refers to any of a number of lipid species that exist either in an uncharged or neutral zwitterionic form at a selected pH. At physiological pH, such lipids include, but are not limited to, phosphotidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phophatidylethanolamines such as 1,2-Dioleoyl-sn-glycero-3-phosphoethanolamine (DOPE), sphingomyelins (SM), ceramides, steroids such as sterols and their derivatives. Neutral lipids may be synthetic or naturally derived.

The term “charged lipid” refers to any of a number of lipid species that exist in either a positively charged or negatively charged form independent of the pH within a useful physiological range e.g. pH ˜3 to pH ˜9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g. DOTAP, DOTMA), dialkyl dimethylaminopropanes, ethyl phosphocholines, dimethylaminoethane carbamoyl sterols (e.g. DC-Chol).

The term “lipid nanoparticle” refers to particles having at least one dimension on the order of nanometers (e.g., 1-1,000 nm) which include one or more of the compounds of structure (I) or other specified cationic lipids. In some embodiments, lipid nanoparticles are included in a formulation that can be used to deliver an active agent or therapeutic agent, such as a nucleic acid (e.g., mRNA) to a target site of interest (e.g., cell, tissue, organ, tumor, and the like). In some embodiments, the lipid nanoparticles of the invention comprise a nucleic acid. Such lipid nanoparticles typically comprise a compound of structure (I) and one or more excipient selected from neutral lipids, charged lipids, steroids and polymer conjugated lipids. In some embodiments, the active agent or therapeutic agent, such as a nucleic acid, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2017/004143, WO 2015/199952, WO 2013/016058 and WO 2013/086373, the full disclosures of which are herein incorporated by reference in their entirety for all purposes.

As used herein, “lipid encapsulated” refers to a lipid nanoparticle that provides an active agent or therapeutic agent, such as a nucleic acid (e.g., mRNA), with full encapsulation, partial encapsulation, or both. In an embodiment, the nucleic acid (e.g., mRNA) is fully encapsulated in the lipid nanoparticle.

As used herein, the term “aqueous solution” refers to a composition comprising water.

“Serum-stable” in relation to nucleic acid-lipid nanoparticles means that the nucleotide is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery of a therapeutic product that can result in a broad exposure of an active agent within an organism. Some techniques of administration can lead to the systemic delivery of certain agents, but not others. Systemic delivery means that a useful, preferably therapeutic, amount of an agent is exposed to most parts of the body. Systemic delivery of lipid nanoparticles can be by any means known in the art including, for example, intravenous, intraarterial, subcutaneous, and intraperitoneal delivery. In some embodiments, systemic delivery of lipid nanoparticles is by intravenous delivery.

“Local delivery,” as used herein, refers to delivery of an active agent directly to a target site within an organism. For example, an agent can be locally delivered by direct injection into a disease site such as a tumor, other target site such as a site of inflammation, or a target organ such as the liver, heart, pancreas, kidney, and the like. Local delivery can also include topical applications or localized injection techniques such as intramuscular, subcutaneous or intradermal injection. Local delivery does not preclude a systemic pharmacological effect.

“Alkyl” refers to a straight or branched hydrocarbon chain radical consisting solely of carbon and hydrogen atoms, which is saturated, having, for example, from one to twenty-four carbon atoms (C₁-C₂₄ alkyl), four to twenty carbon atoms (C₄-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond, e.g., methyl, ethyl, n-propyl, 1 methylethyl (iso propyl), n-butyl, n-pentyl, 1,1-dimethylethyl (t butyl), 3-methylhexyl, 2-methylhexyl, and the like. Unless stated otherwise specifically in the specification, an alkyl group is optionally substituted.

“Alkoxy” refers to a radical of the formula —ORa where Rₐ is an alkyl radical as defined above containing one to twelve carbon atoms. Unless stated otherwise specifically in the specification, an alkoxy group is optionally substituted.

“Alkylaminyl” refers to a radical of the formula —NHRa or —NRaRa where each Rₐ is, independently, an alkyl radical as defined above containing one to twelve carbon atoms. Unless stated otherwise specifically in the specification, an alkylaminyl group is optionally substituted.

“Alkenyl” refers to a straight or branched hydrocarbon chain radical consisting solely of carbon and hydrogen atoms, which contains one or more carbon-carbon double bonds, and having, for example, from two to twenty-four carbon atoms (C₂-C₂₄ alkenyl), four to twenty carbon atoms (C₄-C₂₀ alkenyl), six to sixteen carbon atoms (C₆-C₁₆ alkenyl), six to nine carbon atoms (C₆-C₉ alkenyl), two to fifteen carbon atoms (C₂-C₁₅ alkenyl), two to twelve carbon atoms (C₂-C₁₂ alkenyl), two to eight carbon atoms (C₂-C₈ alkenyl) or two to six carbon atoms (C₂-C₆ alkenyl) and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group is optionally substituted.

“Alkylene” or “alkylene chain” refers to a straight or branched divalent hydrocarbon chain linking the rest of the molecule to a radical group, consisting solely of carbon and hydrogen, which is saturated, and having, for example, from one to twenty-four carbon atoms (C₁-C₂₄ alkylene), two to twenty-four carbon atoms (C₂-C₂₄ alkylene), one to fifteen carbon atoms (C₁-C₁₅ alkylene), one to twelve carbon atoms (C₁-C₁₂ alkylene), one to eight carbon atoms (C₁-C₅ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted.

“Alkenylene” or “alkenylene chain” refers to a straight or branched divalent hydrocarbon chain linking the rest of the molecule to a radical group, consisting solely of carbon and hydrogen, which contains one or more carbon-carbon double bonds, and having, for example, from two to twenty-four carbon atoms (C₂-C₂₄ alkenylene), two to fifteen carbon atoms (C₂-C₁₅ alkenylene), two to twelve carbon atoms (C₂-C₁₂ alkenylene), two to eight carbon atoms (C₂-C₅ alkenylene), two to six carbon atoms (C₂-C₆ alkenylene) or two to four carbon atoms (C₂-C₄ alkenylene), e.g., ethenylene, propenylene, n-butenylene, and the like. The alkenylene chain is attached to the rest of the molecule through a single or double bond and to the radical group through a single or double bond. The points of attachment of the alkenylene chain to the rest of the molecule and to the radical group can be through one carbon or any two carbons within the chain. Unless stated otherwise specifically in the specification, an alkenylene chain may be optionally substituted.

“Aryl” refers to a carbocyclic ring system radical comprising hydrogen, 6 to 18 carbon atoms and at least one aromatic ring. For purposes of this invention, the aryl radical is a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R_{b}—R_{c} where R_{b} is an alkylene or alkenylene as defined above and R_{c} is one or more aryl radicals as defined above, for example, benzyl, diphenylmethyl and the like. Unless stated otherwise specifically in the specification, an aralkyl group is optionally substituted.

“Heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

“Heteroaryl” refers to a 5- to 14-membered ring system radical comprising hydrogen atoms, one to thirteen ring carbon atoms, one to six ring heteroatoms selected from the group consisting of nitrogen, oxygen and sulfur, and at least one aromatic ring comprising a heteroatom. For purposes of embodiments of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group is optionally substituted.

The term “substituted” used herein means any of the above groups (e.g. alkyl, alkenyl, alkylene, alkenylene, aryl and aralkyl,) wherein at least one hydrogen atom is replaced by a bond to a non-hydrogen atom such as, but not limited to: a halogen atom such as F, Cl, Br, or I; oxo groups (═O); hydroxyl groups (—OH); C₁-C₁₂ alkyl groups; cycloalkyl groups; —(C═O)OR′; —O(C═O)R′; —C(═O)R′; —OR′; —S(O)ₓR′; —S—SR′;
C(═O)SR′; —SC(═O)R′; —NR′R′; —NR′C(═O)R′; —C(═O)NR′R′; —NR′C(═O)NR′R′;
OC(═O)NR′R′; —NR′C(═O)OR′; —NR′S(O)ₓNR′R′; —NR′S(O)ₓR′; and —S(O)ₓNR′R′, wherein: R′ is, at each occurrence, independently H, C₁-C₁₅ alkyl or cycloalkyl, and x is 0, 1 or 2. In some embodiments the substituent is a C₁-C₁₂ alkyl group. In other embodiments, the substituent is a cycloalkyl group. In other embodiments, the substituent is a halo group, such as fluoro. In other embodiments, the substituent is an oxo (═O) group. In other embodiments, the substituent is a hydroxyl group. In other embodiments, the substituent is an alkoxy group (—OR′). In other embodiments, the substituent is a carboxyl group. In other embodiments, the substituent is an amine group (—NR′R′).

“Optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

“Prodrug” is meant to indicate a compound that may be converted under physiological conditions or by solvolysis to a biologically active compound of structure (I). Thus, the term “prodrug” refers to a metabolic precursor of a compound of structure (I) that is pharmaceutically acceptable. A prodrug may be inactive when administered to a subject in need thereof, but is converted in vivo to an active compound of structure (I). Prodrugs are typically rapidly transformed in vivo to yield the parent compound of structure (I), for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., A.C.S. Symposium Series, Vol. 14, and in Bioreversible Carriers in Drug Design, Ed. Edward B. Roche, American Pharmaceutical Association and Pergamon Press, 1987.

The term “prodrug” is also meant to include any covalently bonded carriers, which release the active compound of structure (I) in vivo when such prodrug is administered to a mammalian subject. Prodrugs of a compound of structure (I) may be prepared by modifying functional groups present in the compound of structure (I) in such a way that the modifications are cleaved, either in routine manipulation or in vivo, to the parent compound of structure (I). Prodrugs include compounds of structure (I) wherein a hydroxy, amino or mercapto group is bonded to any group that, when the prodrug of the compound of structure (I) is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of structure (I) and the like.

Embodiments of the invention disclosed herein are also meant to encompass all pharmaceutically acceptable compounds of the compound of structure (I) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labelled compounds of structure (I) or (II), for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, 150 and ¹³N, can be useful in Positron Emission Topography (PET) studies for examining substrate receptor occupancy. Isotopically-labeled compounds of structure (I) can generally be prepared by conventional techniques known to those skilled in the art or by processes analogous to those described in the Preparations and Examples as set out below using an appropriate isotopically-labeled reagent in place of the non-labeled reagent previously employed.

Embodiments of the invention disclosed herein are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments of the invention include compounds produced by a process comprising administering a compound of this invention to a mammal for a period of time sufficient to yield a metabolic product thereof. Such products are typically identified by administering a radiolabeled compound of structure (I) in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of structure (I). As used herein, the term “solvate” refers to an aggregate that comprises one or more molecules of a compound of structure (I) with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. In some embodiments, the compound of structure (I) may exist as a true solvate, while in other cases, the compound of structure (I) may merely retain adventitious water or be a mixture of water plus some adventitious solvent.

A “pharmaceutical composition” refers to a formulation of a compound of structure (I) and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound of structure (I) which, when administered to a mammal, preferably a human, is sufficient to effect treatment in the mammal, preferably a human. The amount of a lipid nanoparticle of embodiments the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; or
(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of structure (I), or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. Embodiments of the present invention are meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

### Compounds

In an aspect, the invention provides novel lipid compounds which are capable of combining with other lipid components such as neutral lipids, charged lipids, steroids and/or polymer conjugated-lipids to form lipid nanoparticles with oligonucleotides. Without wishing to be bound by theory, it is thought that these lipid nanoparticles shield oligonucleotides from degradation in the serum and provide for effective delivery of oligonucleotides to cells in vitro and in vivo.

In one embodiment, the compounds have the following structure (I):

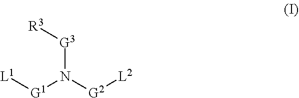

or a pharmaceutically acceptable salt, prodrug or stereoisomer thereof, wherein:
L¹ is —NR^{a}C(═O)R¹ or —C(═O)NR^{b}R^{c};
L² is —NR^{d}C(═O)R² or —C(═O)NR^{e}R^{f};
G¹ and G² are each independently C₂-C₁₂ alkylene, or C₂-C₁₂ alkenylene;
G³ is C₁-C₂₄ alkylene, or C₂-C₂₄ alkenylene;
R^{a}, R^{b}, R^{d} and R^{e} are each independently H, C₁-C₁₆ alkyl or C₂-C₁₆ alkenyl;
R^{c} and R^{f} are each independently C₁-C₁₆ alkyl or C₂-C₁₆ alkenyl;
R¹ and R² are each independently C₆-C₂₄ alkyl or C₆-C₂₄ alkenyl;
R³ is H, —OH, CN, —N(R⁴)R⁵; —C(═O)N(R⁴)R⁵; —N(R⁴)C(═O)R⁵; —N(R⁴)C(═O)OR⁵; —C(═O)OR⁶, —OC(═O)R⁶, —OR⁷, heteroaryl or aryl;
R⁴ and R⁵ are each independently is H, C₁-C₁₂ alkyl, C₃-C₆ cycloalkyl or C₃-C₆ cycloalkenyl, or R⁴ and R⁵, together with the nitrogen or carbon atom to which they are bound, form a 5 to 7-membered heterocyclic ring;
R⁶ is H, C₁-C₁₂ alkyl, C₂-C₁₂ alkenyl or aralkyl;
R⁷ is C₁-C₁₂ alkyl optionally substituted with hydroxyl or alkoxy; and
wherein each alkyl, alkenyl, alkylene, alkenylene, aryl and aralkyl is independently substituted or unsubstituted unless otherwise specified. In certain embodiments, at least one alkyl, alkenyl, alkylene, alkenylene, C₃-C₆ cycloalkyl or C₃-C₆ cycloalkenyl, aryl or aralkyl is substituted with one or more fluorine and/or one or more oxo and/or one or more NH₂ and/or one or more alkylaminyl.

In one embodiment, the compounds have the following structure (I):

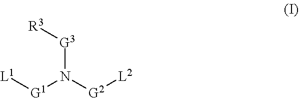

or a pharmaceutically acceptable salt, prodrug or stereoisomer thereof, wherein:
L¹ is —NR^{a}C(═O)R¹ or —C(═O)NR^{b}R^{c};
L² is —NR^{d}C(═O)R² or —C(═O)NR^{e}R^{f};
G¹ and G² are each independently C₁-C₁₂ alkylene, or C₂-C₁₂ alkenylene;
G³ is C₁-C₂₄ alkylene, or C₂-C₂₄ alkenylene;
R^{a}, R^{b}, R^{d} and R^{e} are each independently H, C₁-C₁₆ alkyl or C₂-C₁₆ alkenyl;
R^{c} and R^{f} are each independently C₁-C₁₆ alkyl or C₂-C₁₆ alkenyl;
R¹ and R² are each independently C₆-C₂₄ alkyl or C₆-C₂₄ alkenyl;
R³ is H, —OH, CN, —N(R⁴)R⁵; —C(═O)N(R⁴)R⁵; —N(R⁴)C(═O)R⁵; —C(═O)OR⁶, —OC(═O)R⁶, or aryl;
R⁴ and R⁵ are each independently is H, C₁-C₁₂ alkyl, or R⁴ and R⁵, together with the nitrogen or carbon atom to which they are bound, form a 5 to 7-membered heterocyclic ring;
R⁶ is H, C₁-C₁₂ alkyl, C₂-C₁₂ alkenyl or aralkyl; and
   wherein each alkyl, alkenyl, alkylene, alkenylene, C₃-C₆ cycloalkyl or C₃-C₆ cycloalkenyl, aryl and aralkyl is independently substituted or unsubstituted unless otherwise specified. In certain embodiments, at least one alkyl, alkenyl, alkylene, alkenylene, C₃-C₆ cycloalkyl or C₃-C₆ cycloalkenyl, aryl or aralkyl is substituted with one or more fluorine and/or one or more oxo and/or one or more NH₂ and/or one or more alkylaminyl.

In certain embodiments, G³ is unsubstituted. In some embodiments, G³ is substituted with one or more fluorine atoms. In more specific embodiments G³ is C₁-C₁₂ alkylene, for example. In some embodiments, G³ is C₁, C₂, C₃, C₄, C₅, C₆, C₇, or C₈ alkylene. For example, in some embodiments G³ is methyl, ethyl, n-propyl, n-butyl, n-pentyl, n-hexyl, n-heptyl, or n-octyl.

In certain embodiments, G¹ and/or G² is unsubstituted. In some embodiments, G¹ and/or G² is substituted with one or more fluorine atoms. In more specific embodiments G¹ and/or G² is C₁-C₁₂ alkylene, for example. In some embodiments, G¹ and/or G² is C₁, C₂, C₃, C₄, C₅, C₆, C₇, or C₈ alkylene.

In some of the foregoing embodiments, the compound has the following structure (IA):

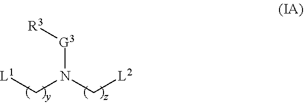

wherein y and z are each independently integers ranging from 2 to 12, for example an integer from 2 to 6, for example 4 or 5. In some certain embodiments, y and z are each integers of 5. In some certain embodiments, y and z are each integers of 6. In some certain embodiments, y and z are each integers of 7. In some certain embodiments, y and z are each integers of 9.

In some of the foregoing embodiments, L¹ is —C(═O)NR^{b}R^{c}, and L² is
—C(═O)NR^{e}R^{f}. In some of the foregoing embodiments, L¹ is —NR^{a}C(═O)R¹ and L² is —NR^{d}C(═O)R².

In other embodiments of the foregoing, the compound having one of the following structures (IB) or (IC):

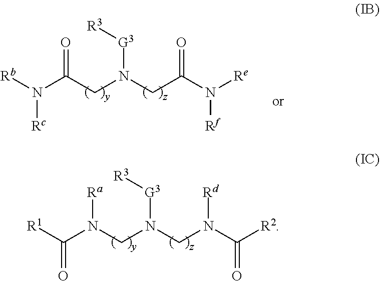

In some of the foregoing embodiments, y and z are each independently an integer ranging from 2 to 10, 2 to 8, from 4 to 10 or from 4 to 7. For example, in some embodiments, y is 4, 5, 6, 7, 8, 9, 10, 11 or 12. In some embodiments, z is 4, 5, 6, 7, 8, 9, 10, 11 or 12. In some certain embodiments, y and z are each integers of 5. In some certain embodiments, y and z are each integers of 6. In some certain embodiments, y and z are each integers of 7. In some certain embodiments, y and z are each integers of 9. In some embodiments, y and z are the same, while in other embodiments y and z are different.

In some of the foregoing embodiments, R¹ or R², or both is branched C₆-C₂₄ alkyl. For example, in some embodiments, R¹ and R² each, independently have the following structure:

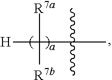

wherein:

R^{7a} and R^{7b} are, at each occurrence, independently either: (a) H or C₁-C₁₂ alkyl, or (b) R^{7a} is H or C₁-C₁₂ alkyl, and R^{7b} together with the carbon atom to which it is bound is taken together with an adjacent R^{7b} and the carbon atom to which it is bound to form a carbon-carbon double bond; and a is an integer from 2 to 12, wherein R^{7a}, R^{7b} and a are each selected such that R¹ and R² are each independently linear or branched and independently comprise from 6 to 20 carbon atoms. For example, in some embodiments a is an integer ranging from 5 to 9 or from 8 to 12.

In some of the foregoing embodiments, at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments, R¹ or R², or both, has one of the following structures:

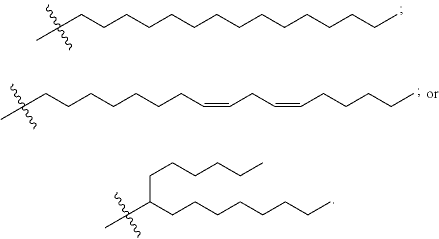 [SMILES: *CCCCCCCCCCCCCCC ; *CCCCCCC/C=C\C/C=C\CCCCC ; *C(CCCCCC)CCCCCCCC]

In different embodiments, R¹ or R², or both, has one of the following structures:

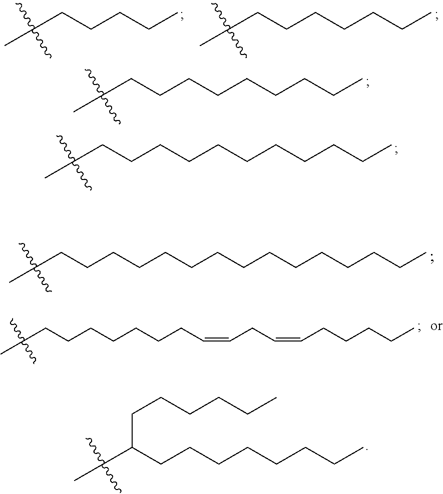

In some of the foregoing embodiments, R^{b}, R^{c}, R^{e} and R^{f} are each independently C₃-C₁₂ alkyl or C₃-C₁₆ alkyl. For example, in some embodiments R^{b}, R^{c}, R^{e} and R^{f} are selected from n-hexyl (—(CH₂)₅CH₃), n-octyl (—(CH₂)₇CH₃), are n-decanyl (—(CH₂)₉CH₃), n-dodecyl (—(CH₂)₁₁CH₃), (—(CH₂)₁₄CH₃), (—(CH₂)₁₅CH₃). For example, in some embodiments R^{b}, R^{c}, R^{e} and R^{f} are n-hexyl (—(CH₂)₅CH₃) and in other embodiments R^{b}, R^{c}, R^{e} and R^{f} are n-octyl (—(CH₂)₇CH₃). In another example in some embodiments R^{b}, R^{c}, R^{e} and R^{f} are n-decanyl (—(CH₂)₉CH₃). In other embodiments, R^{b}, R^{c}, R^{e} and R^{f} are n-dodecyl (—(CH₂)₁₁CH₃).

In some embodiments, R^{a} and R^{d} are each independently C₁-C₁₂ alkyl or C₂-C₁₂ alkenyl and R¹ and R² are each independently C₆-C₁₈ alkyl or C₆-C₁₈ alkenyl. For example in some embodiments R^{a} and R^{d} are n-hexyl (—(CH₂)₅CH₃) and R¹ and R² are dodecapentyl (—(CH₂)₁₄CH₃). In another example in some embodiments R^{a} and R^{d} are n-hexyl and R¹ and R² are (6Z, 9Z)-heptadeca-6,9-diene (—(CH₂)₇CHCHCH₂CHCH(CH₂)₄CH₃). In yet another example in some embodiments R^{a} and R^{d} are n-hexyl and R¹ and R² are 7-pentadecane (—CH((CH₂)₅CH₃)((CH₂)₇CH₃)).

In some embodiment, R³ is H.

In other embodiments, R³ is —OH.

In some different embodiments, R³ is —C(═O)OR⁶, for example in some of these embodiments R⁶ is C₁-C₁₈ alkyl. In another example in some embodiments, R⁶ is C₁-C₁₂ branched alkyl. In yet another example in some embodiments, R⁶ is C₁-C₆ alkyl. In another example in some embodiments, R⁶ is C₁-C₂ alkyl. In some certain embodiments, R⁶ is an ethyl. In some certain embodiments, R⁶ is 5-methyl dodecyl.

In other embodiments, R³ is —N(R⁴)R⁵, for example in some of these embodiments R⁴ and R⁵ are each independently C₁-C₁₂ alkyl, optionally substituted with hydroxyl. In another example in some embodiments R⁴ and R⁵ are each independently C₁-C₆ alkyl, optionally substituted with hydroxyl. In yet another example in some embodiments R⁴ and R⁵ are each independently C₁-C₂ alkyl, optionally substituted with hydroxyl. In some certain embodiments, R⁴ and R⁵ are each methyl. In some certain embodiments, R⁴ and R⁵ are each n-hexyl.

In other embodiments, R³ is —C(═O)N(R⁴)R⁵ or R³ is —N(R⁴)C(═O)R⁵. In some of these embodiments, one of R⁴ or R⁵ is H and the other one of R⁴ or R⁵ is C₁-C₁₂ alkyl. In some embodiments, one of R⁴ or R⁵ is H and the other one of R⁴ or R⁵ is C₁-C₁₂ alkyl substituted with hydroxyl. In other embodiments, both R⁴ and R⁵ are C₁-C₁₂ alkyl or C₁-C₆ alkyl. In some embodiments, both R⁴ and R⁵ are C₁-C₁₂ alkyl or C₁-C₆ alkyl substituted with hydroxyl. In some certain embodiments, one of R⁴ or R⁵ is H and the other one of R⁴ or R⁵ is n-decyl. In some certain embodiments, one of R⁴ or R⁵ is H and the other one of R⁴ or R⁵ is n-tridecyl.

In some embodiments, R³ is aryl. In some embodiments, R³ is substituted aryl. In some certain embodiments, R³ is a phenyl.

In other embodiments, R³ is —N(R⁴)R⁵, and one of R⁴ and R⁵ is C₃-C₆ cycloalkyl or C₃-C₆ cycloalkenyl. For example, in some embodiments R³ is —N(R⁴)R⁵, R⁴ is H and R⁵ is C₃-C₆ cycloalkenyl.

In different embodiments, R³ is —OR⁷ and R⁷ is C₁-C₆ alkyl substituted with OH or OCH₃.

In other embodiments, R³ is —N(R⁴)C(═O)OR⁵, wherein R⁴ is H and R⁵ is C₁-C₆ alkyl.

In still more embodiments, R³ is heteroaryl, for example imidazolyl.

In some more specific embodiments, R³ has one of the following structures:

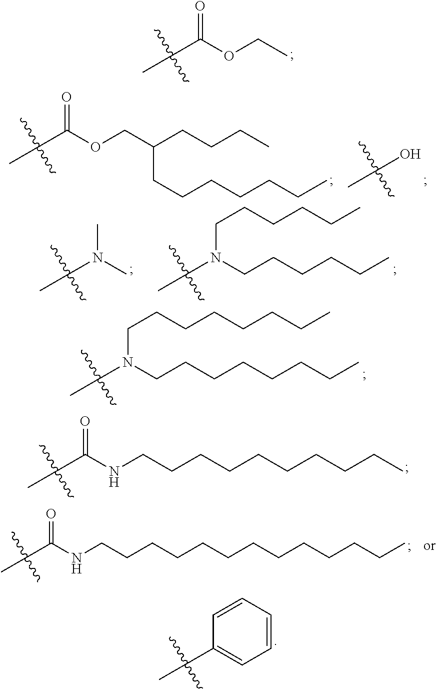

In some more specific embodiments, R³ is H or has one of the following structures:

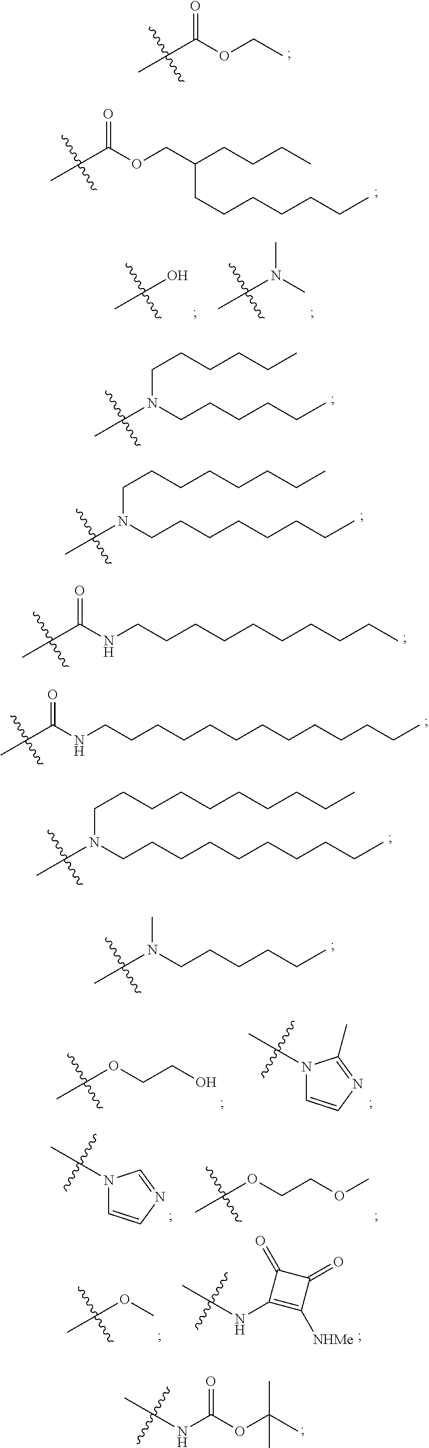

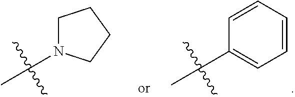 [SMILES: *N1CCCC1 ; *c1ccccc1]

In various different embodiments, the compound has one of the structures set forth in Table 1 below.

TABLE 1
Representative Compounds

| No. | Structure |
|---|---|
| I-1 | 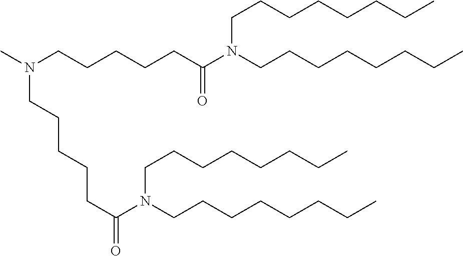 [SMILES: CCCCCCCCN(CCCCCCCC)C(=O)CCCCCN(C)CCCCCC(=O)N(CCCCCCCC)CCCCCCCC] |
| I-2 | 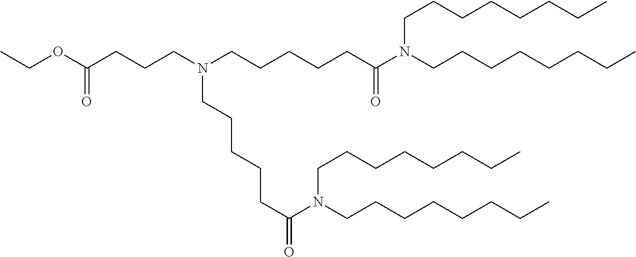 [SMILES: CCCCCCCCN(CCCCCCCC)C(=O)CCCCCN(CCCCCC(=O)N(CCCCCCCC)CCCCCCCC)CCCC(=O)OCC] |
| I-3 | 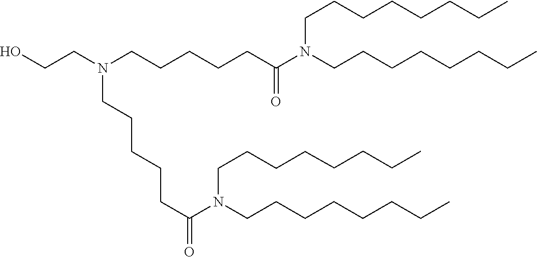 [SMILES: CCCCCCCCN(CCCCCCCC)C(=O)CCCCCN(CCO)CCCCCC(=O)N(CCCCCCCC)CCCCCCCC] |
| I-4 | 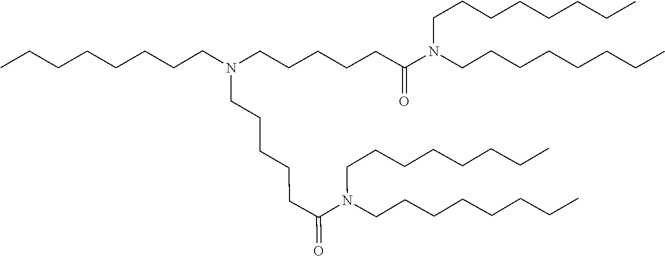 [SMILES: CCCCCCCCN(CCCCCC(=O)N(CCCCCCCC)CCCCCCCC)CCCCCC(=O)N(CCCCCCCC)CCCCCCCC] |
| I-5 | 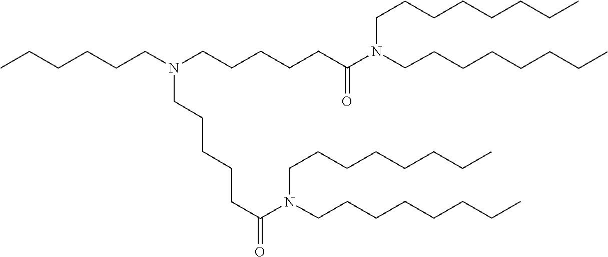 [SMILES: CCCCCCCCN(CCCCCCCC)C(=O)CCCCCN(CCCCCC)CCCCCC(=O)N(CCCCCCCC)CCCCCCCC] |
| I-6 | 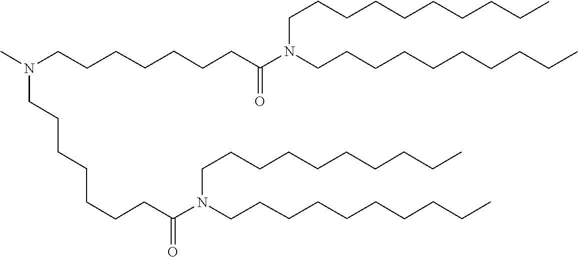 [SMILES: CCCCCCCCCCN(CCCCCCCCCC)C(=O)CCCCCCCN(C)CCCCCCCC(=O)N(CCCCCCCCCC)CCCCCCCCCC] |
| I-7 | 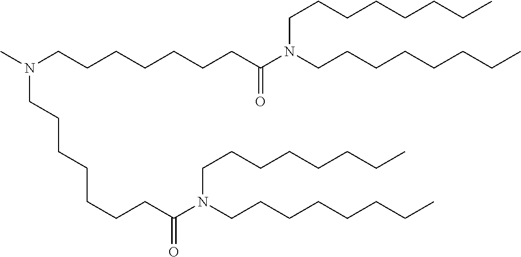 [SMILES: CCCCCCCCN(CCCCCCCC)C(=O)CCCCCCCN(C)CCCCCCCC(=O)N(CCCCCCCC)CCCCCCCC] |
| I-8 | 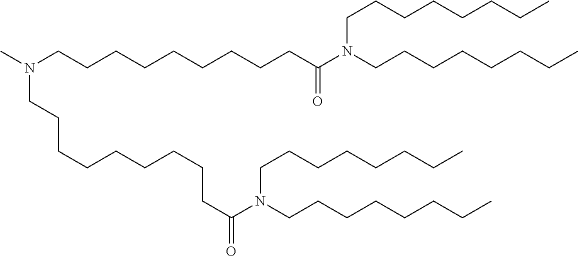 [SMILES: CCCCCCCCN(CCCCCCCC)C(=O)CCCCCCCCCN(C)CCCCCCCCCC(=O)N(CCCCCCCC)CCCCCCCC] |
| I-9 | 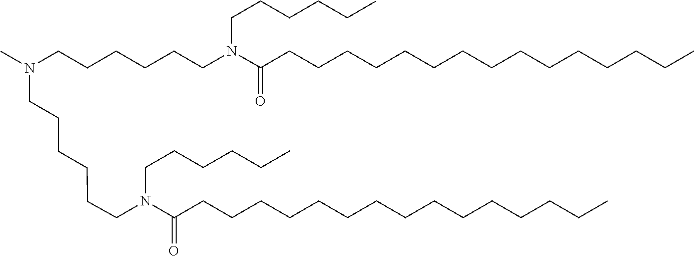 [SMILES: CCCCCCCCCCCCCCCC(=O)N(CCCCCC)CCCCCCN(C)CCCCCCN(CCCCCC)C(=O)CCCCCCCCCCCCCCC] |
| I-10 | 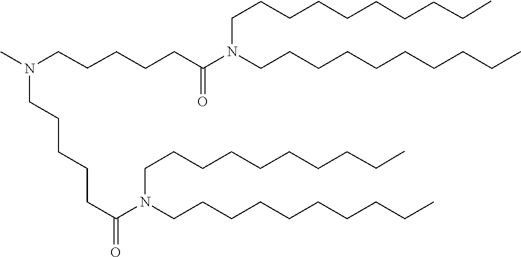 [SMILES: CCCCCCCCCCN(CCCCCCCCCC)C(=O)CCCCCN(C)CCCCCC(=O)N(CCCCCCCCCC)CCCCCCCCCC] |
| I-11 | 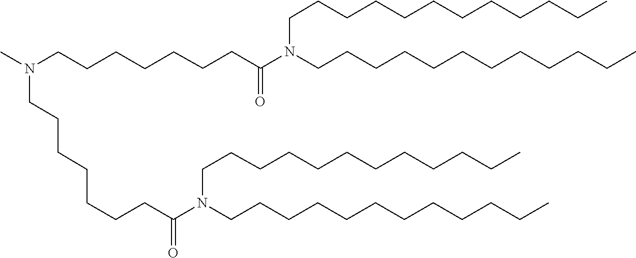 [SMILES: CCCCCCCCCCCCN(CCCCCCCCCCCC)C(=O)CCCCCCCN(C)CCCCCCCC(=O)N(CCCCCCCCCCCC)CCCCCCCCCCCC] |
| I-12 | 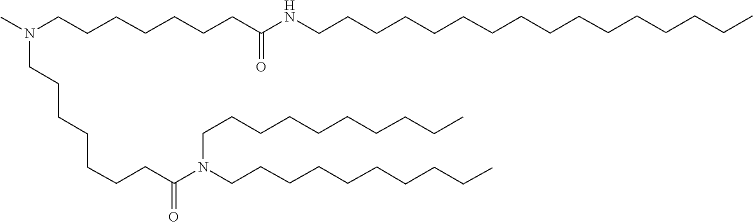 [SMILES: CCCCCCCCCCCCCCCCNC(=O)CCCCCCCN(C)CCCCCCCC(=O)N(CCCCCCCCCC)CCCCCCCCCC] |
| I-13 | 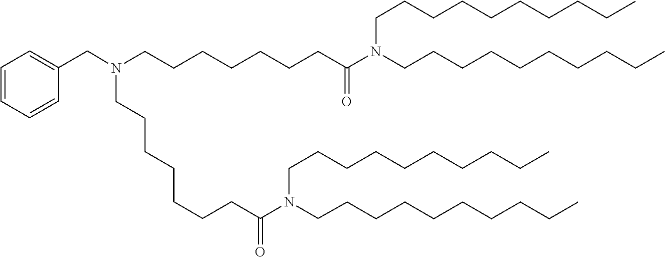 [SMILES: CCCCCCCCCCN(CCCCCCCCCC)C(=O)CCCCCCCN(CCCCCCCC(=O)N(CCCCCCCCCC)CCCCCCCCCC)Cc1ccccc1] |
| I-14 | 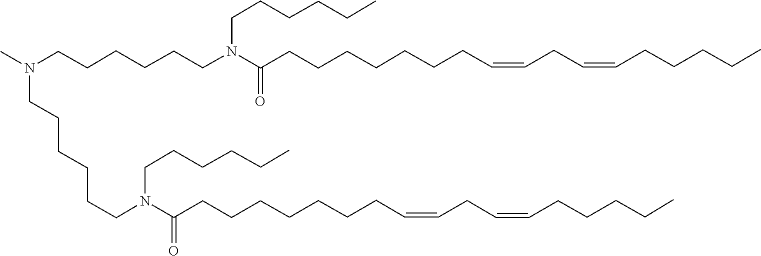 [SMILES: CCCCC/C=C\C/C=C\CCCCCCCC(=O)N(CCCCCC)CCCCCCN(C)CCCCCCN(CCCCCC)C(=O)CCCCCCC/C=C\C/C=C\CCCCC] |
| I-15 | 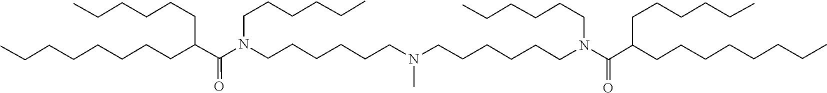 [SMILES: CCCCCCCCC(CCCCCC)C(=O)N(CCCCCC)CCCCCCN(C)CCCCCCN(CCCCCC)C(=O)C(CCCCCC)CCCCCCCC] |
| I-16 | 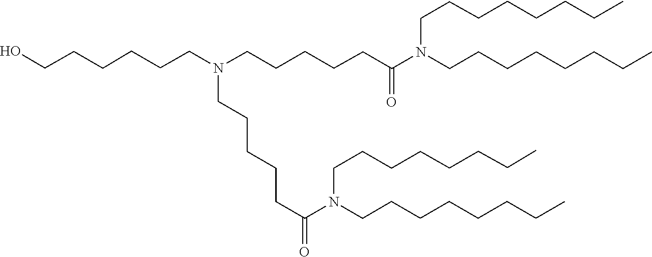 [SMILES: CCCCCCCCN(CCCCCCCC)C(=O)CCCCCN(CCCCCCO)CCCCCC(=O)N(CCCCCCCC)CCCCCCCC] |
| I-17 | 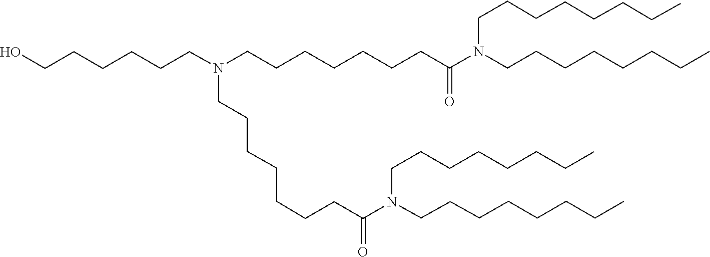 [SMILES: CCCCCCCCN(CCCCCCCC)C(=O)CCCCCCCN(CCCCCCO)CCCCCCCC(=O)N(CCCCCCCC)CCCCCCCC] |
| I-18 | 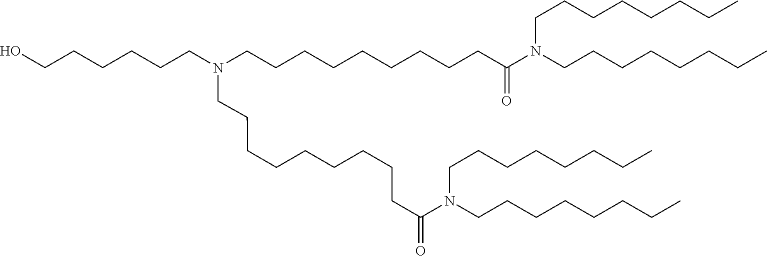 [SMILES: CCCCCCCCN(CCCCCCCC)C(=O)CCCCCCCCCN(CCCCCCO)CCCCCCCCCC(=O)N(CCCCCCCC)CCCCCCCC] |
| I-19 | 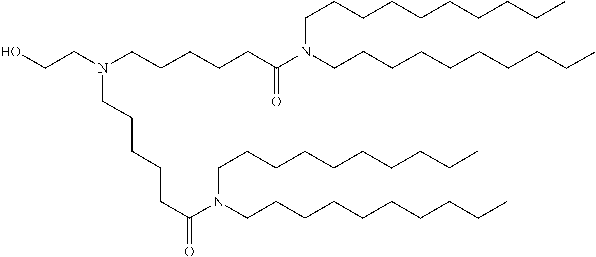 [SMILES: CCCCCCCCCCN(CCCCCCCCCC)C(=O)CCCCCN(CCO)CCCCCC(=O)N(CCCCCCCCCC)CCCCCCCCCC] |
| I-20 | 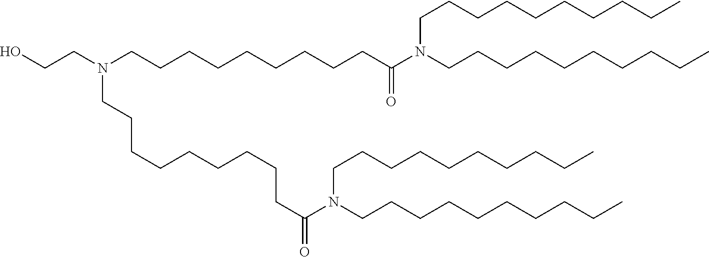 [SMILES: CCCCCCCCCCN(CCCCCCCCCC)C(=O)CCCCCCCCCN(CCO)CCCCCCCCCC(=O)N(CCCCCCCCCC)CCCCCCCCCC] |
| I-21 | 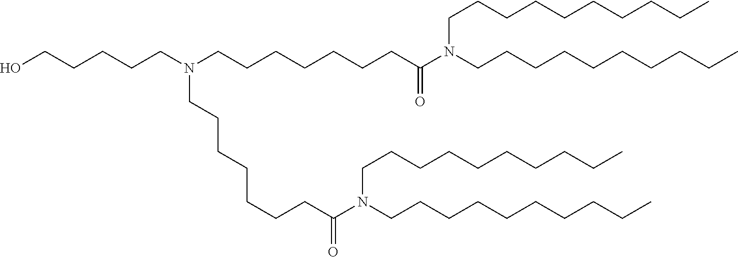 [SMILES: CCCCCCCCCCN(CCCCCCCCCC)C(=O)CCCCCCCN(CCCCCO)CCCCCCCC(=O)N(CCCCCCCCCC)CCCCCCCCCC] |
| I-22 | 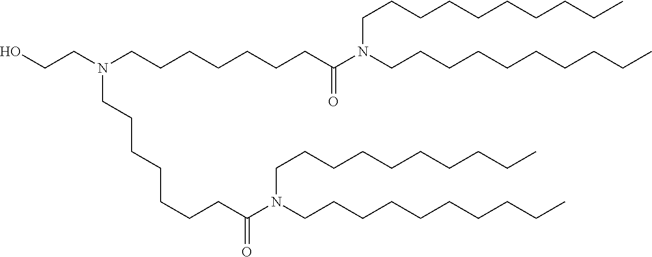 [SMILES: CCCCCCCCCCN(CCCCCCCCCC)C(=O)CCCCCCCN(CCO)CCCCCCCC(=O)N(CCCCCCCCCC)CCCCCCCCCC] |
| I-23 | 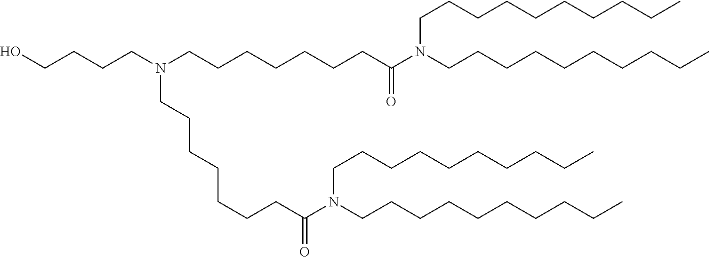 [SMILES: CCCCCCCCCCN(CCCCCCCCCC)C(=O)CCCCCCCN(CCCCO)CCCCCCCC(=O)N(CCCCCCCCCC)CCCCCCCCCC] |
| I-24 | 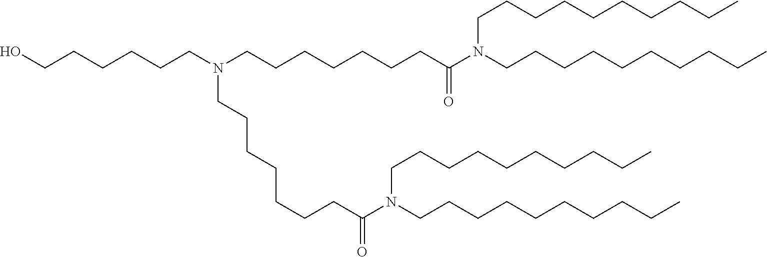 [SMILES: CCCCCCCCCCN(CCCCCCCCCC)C(=O)CCCCCCCN(CCCCCCO)CCCCCCCC(=O)N(CCCCCCCCCC)CCCCCCCCCC] |
| I-25 | 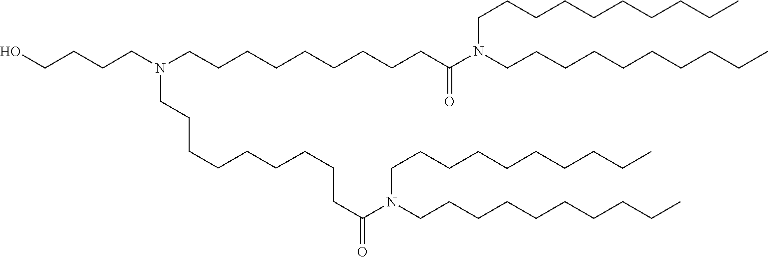 [SMILES: CCCCCCCCCCN(CCCCCCCCCC)C(=O)CCCCCCCCCN(CCCCO)CCCCCCCCCC(=O)N(CCCCCCCCCC)CCCCCCCCCC] |
| I-26 | 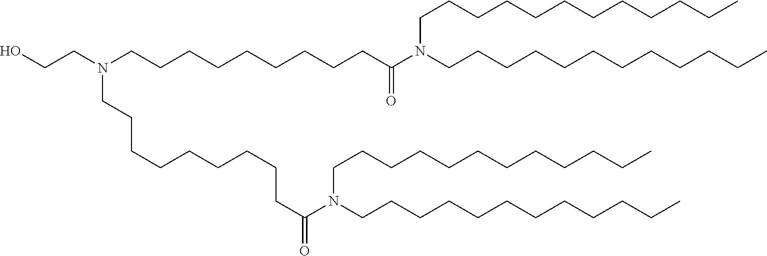 [SMILES: CCCCCCCCCCCCN(CCCCCCCCCCCC)C(=O)CCCCCCCCCN(CCO)CCCCCCCCCC(=O)N(CCCCCCCCCCCC)CCCCCCCCCCCC] |
| I-27 | 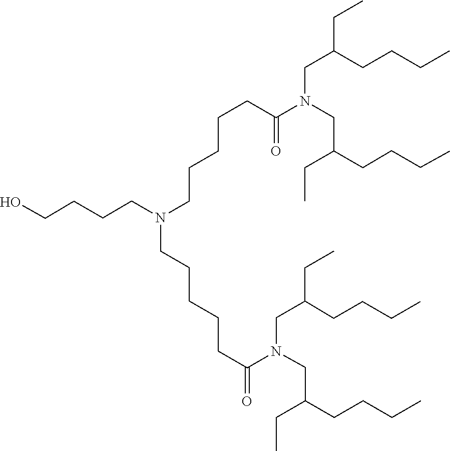 [SMILES: CCCCC(CC)CN(CC(CC)CCCC)C(=O)CCCCCN(CCCCO)CCCCCC(=O)N(CC(CC)CCCC)CC(CC)CCCC] |
| I-28 | 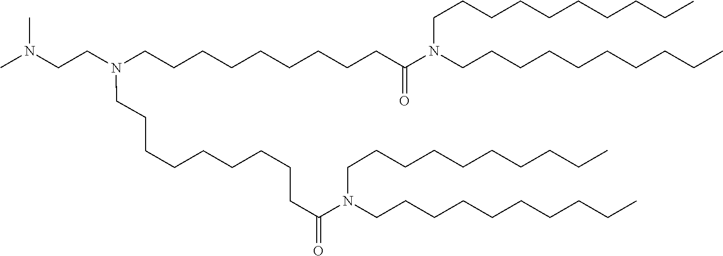 [SMILES: CCCCCCCCCCN(CCCCCCCCCC)C(=O)CCCCCCCCCN(CCCCCCCCCC(=O)N(CCCCCCCCCC)CCCCCCCCCC)CCN(C)C] |
| I-29 | 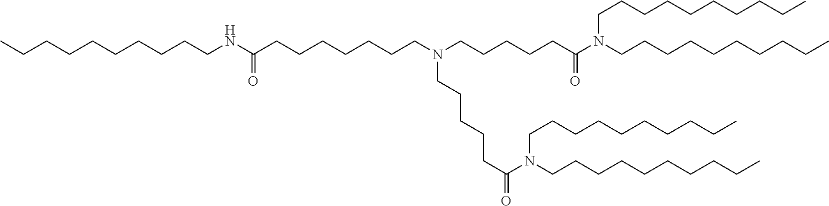 [SMILES: CCCCCCCCCCNC(=O)CCCCCCCN(CCCCCC(=O)N(CCCCCCCCCC)CCCCCCCCCC)CCCCCC(=O)N(CCCCCCCCCC)CCCCCCCCCC] |
| I-30 | 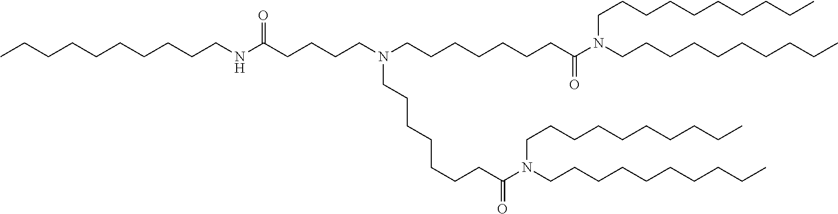 [SMILES: CCCCCCCCCCNC(=O)CCCCN(CCCCCCCC(=O)N(CCCCCCCCCC)CCCCCCCCCC)CCCCCCCC(=O)N(CCCCCCCCCC)CCCCCCCCCC] |
| I-31 | 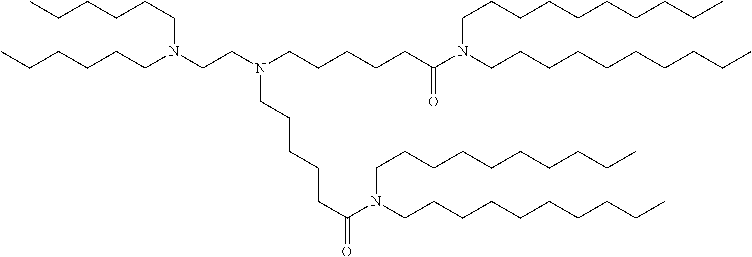 [SMILES: CCCCCCCCCCN(CCCCCCCCCC)C(=O)CCCCCN(CCCCCC(=O)N(CCCCCCCCCC)CCCCCCCCCC)CCN(CCCCCC)CCCCCC] |
| I-32 | 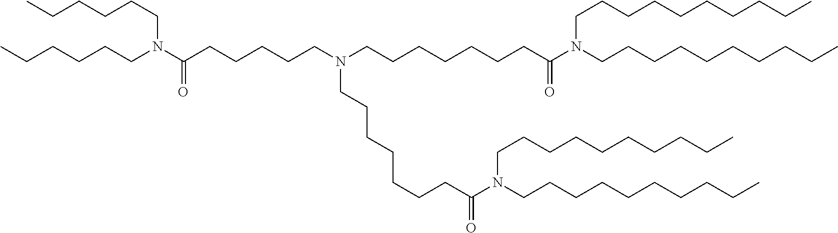 [SMILES: CCCCCCCCCCN(CCCCCCCCCC)C(=O)CCCCCCCN(CCCCCCCC(=O)N(CCCCCCCCCC)CCCCCCCCCC)CCCCCC(=O)N(CCCCCC)CCCCCC] |
| I-33 | 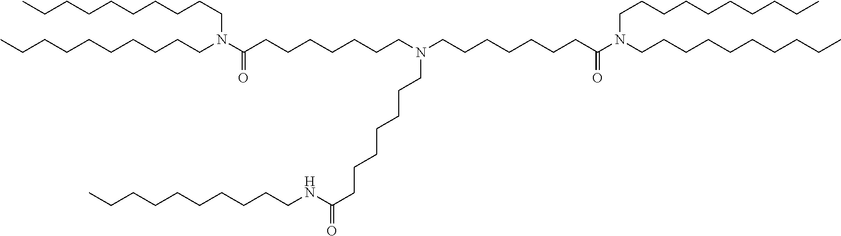 [SMILES: CCCCCCCCCCNC(=O)CCCCCCCN(CCCCCCCC(=O)N(CCCCCCCCCC)CCCCCCCCCC)CCCCCCCC(=O)N(CCCCCCCCCC)CCCCCCCCCC] |
| I-34 | 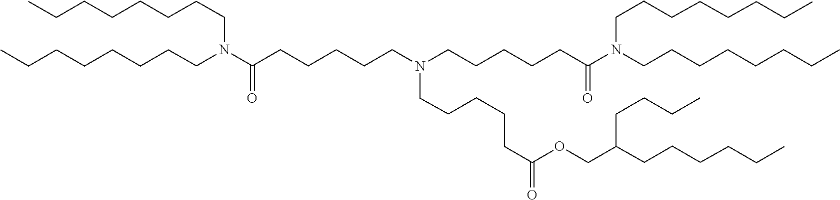 [SMILES: CCCCCCCCN(CCCCCCCC)C(=O)CCCCCN(CCCCCC(=O)OCC(CCCC)CCCCCC)CCCCCC(=O)N(CCCCCCCC)CCCCCCCC] |
| I-35 | 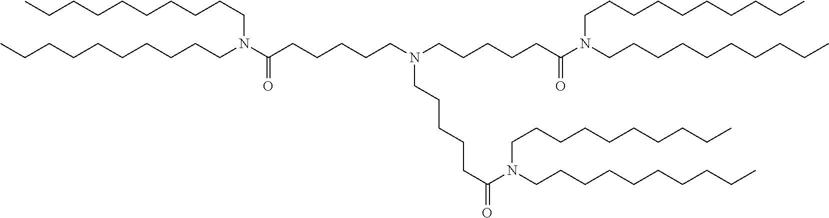 [SMILES: CCCCCCCCCCN(CCCCCCCCCC)C(=O)CCCCCN(CCCCCC(=O)N(CCCCCCCCCC)CCCCCCCCCC)CCCCCC(=O)N(CCCCCCCCCC)CCCCCCCCCC] |
| I-36 | 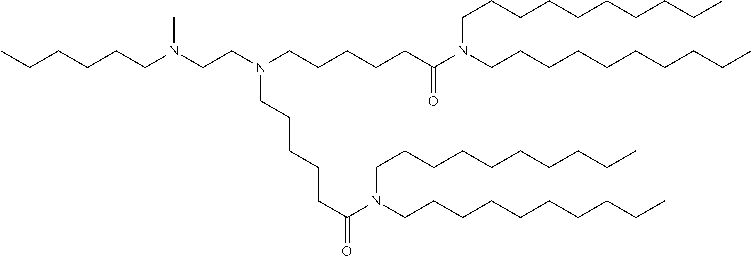 [SMILES: CCCCCCCCCCN(CCCCCCCCCC)C(=O)CCCCCN(CCCCCC(=O)N(CCCCCCCCCC)CCCCCCCCCC)CCN(C)CCCCCC] |
| I-37 | 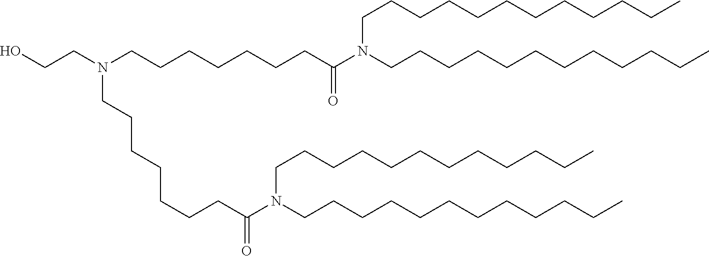 [SMILES: CCCCCCCCCCCCN(CCCCCCCCCCCC)C(=O)CCCCCCCN(CCO)CCCCCCCC(=O)N(CCCCCCCCCCCC)CCCCCCCCCCCC] |
| I-38 | 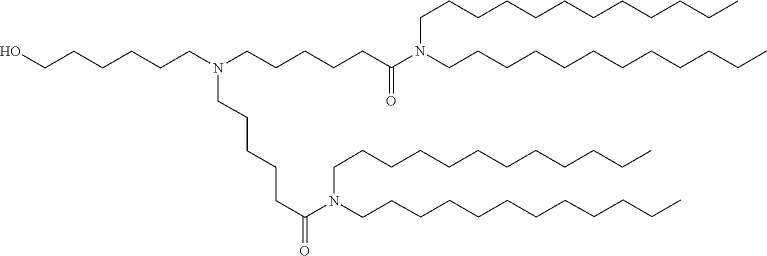 [SMILES: CCCCCCCCCCCCN(CCCCCCCCCCCC)C(=O)CCCCCN(CCCCCCO)CCCCCC(=O)N(CCCCCCCCCCCC)CCCCCCCCCCCC] |
| I-39 | 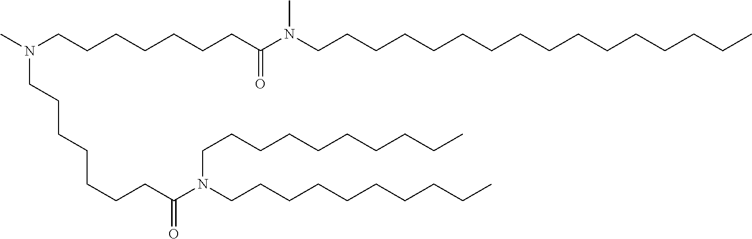 [SMILES: CCCCCCCCCCCCCCCCN(C)C(=O)CCCCCCCN(C)CCCCCCCC(=O)N(CCCCCCCCCC)CCCCCCCCCC] |
| I-40 | 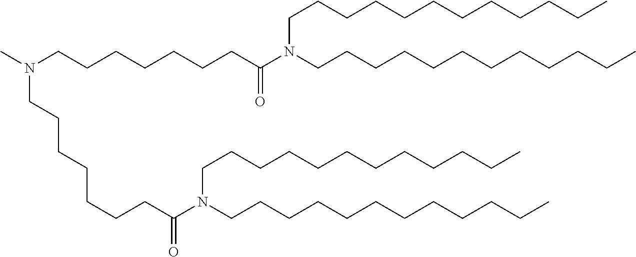 [SMILES: CCCCCCCCCCCCN(CCCCCCCCCCCC)C(=O)CCCCCCCN(C)CCCCCCCC(=O)N(CCCCCCCCCCCC)CCCCCCCCCCCC] |
| I-41 | 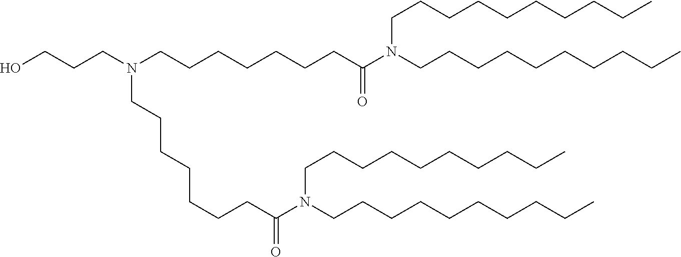 [SMILES: CCCCCCCCCCN(CCCCCCCCCC)C(=O)CCCCCCCN(CCCO)CCCCCCCC(=O)N(CCCCCCCCCC)CCCCCCCCCC] |
| I-42 | 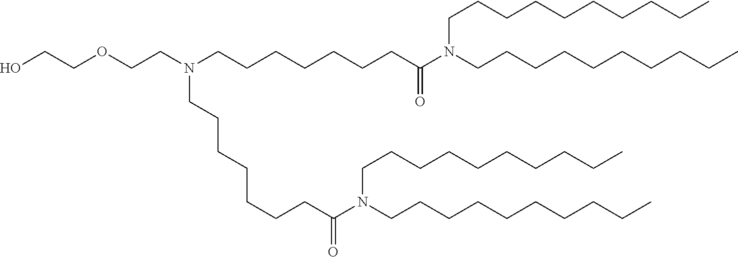 [SMILES: CCCCCCCCCCN(CCCCCCCCCC)C(=O)CCCCCCCN(CCCCCCCC(=O)N(CCCCCCCCCC)CCCCCCCCCC)CCOCCO] |
| I-43 | 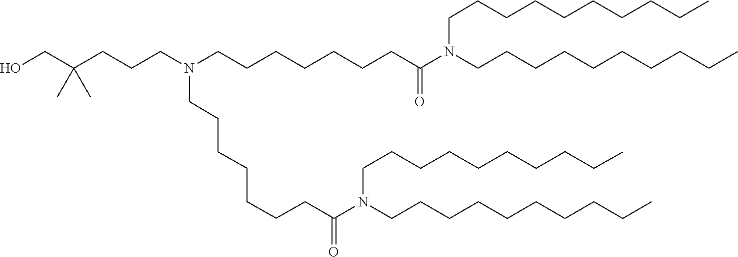 [SMILES: CCCCCCCCCCN(CCCCCCCCCC)C(=O)CCCCCCCN(CCCCCCCC(=O)N(CCCCCCCCCC)CCCCCCCCCC)CCCC(C)(C)CO] |
| I-44 | 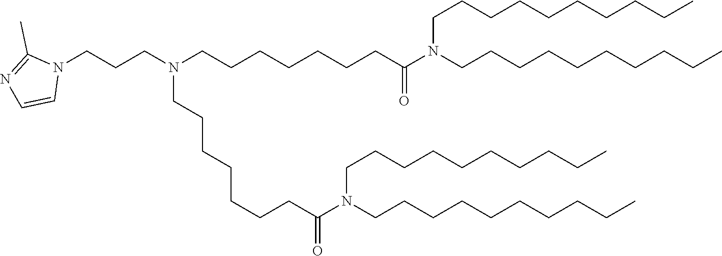 [SMILES: CCCCCCCCCCN(CCCCCCCCCC)C(=O)CCCCCCCN(CCCCCCCC(=O)N(CCCCCCCCCC)CCCCCCCCCC)CCCn1ccnc1C] |
| I-45 | 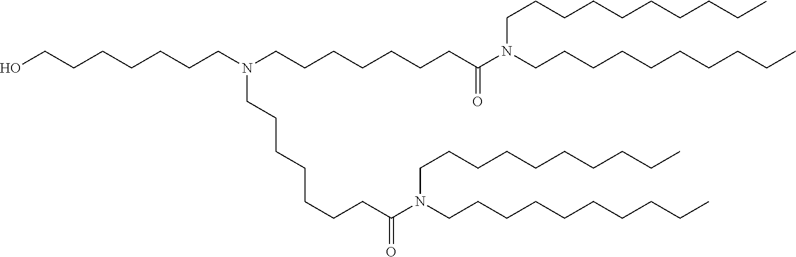 [SMILES: CCCCCCCCCCN(CCCCCCCCCC)C(=O)CCCCCCCN(CCCCCCCO)CCCCCCCC(=O)N(CCCCCCCCCC)CCCCCCCCCC] |
| I-46 | 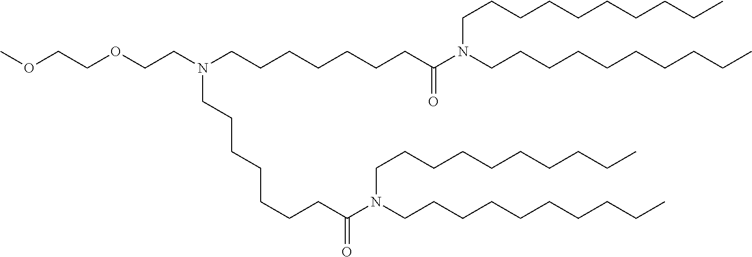 [SMILES: CCCCCCCCCCN(CCCCCCCCCC)C(=O)CCCCCCCN(CCCCCCCC(=O)N(CCCCCCCCCC)CCCCCCCCCC)CCOCCOC] |
| I-47 | 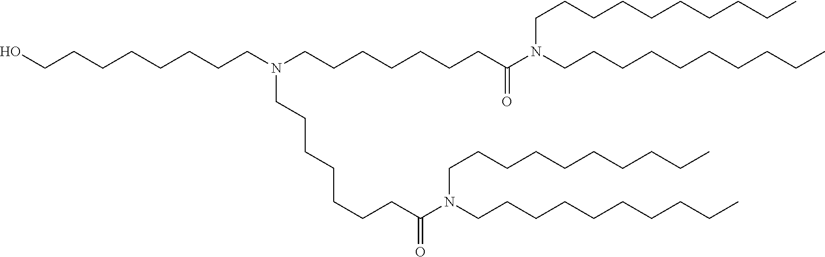 [SMILES: CCCCCCCCCCN(CCCCCCCCCC)C(=O)CCCCCCCN(CCCCCCCCO)CCCCCCCC(=O)N(CCCCCCCCCC)CCCCCCCCCC] |
| I-48 | 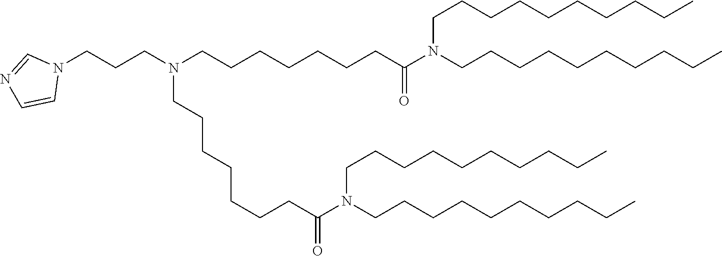 [SMILES: CCCCCCCCCCN(CCCCCCCCCC)C(=O)CCCCCCCN(CCCCCCCC(=O)N(CCCCCCCCCC)CCCCCCCCCC)CCCn1ccnc1] |
| I-49 | 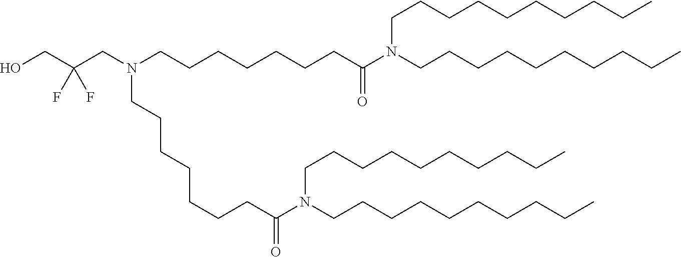 [SMILES: CCCCCCCCCCN(CCCCCCCCCC)C(=O)CCCCCCCN(CCCCCCCC(=O)N(CCCCCCCCCC)CCCCCCCCCC)CC(F)(F)CO] |
| I-50 | 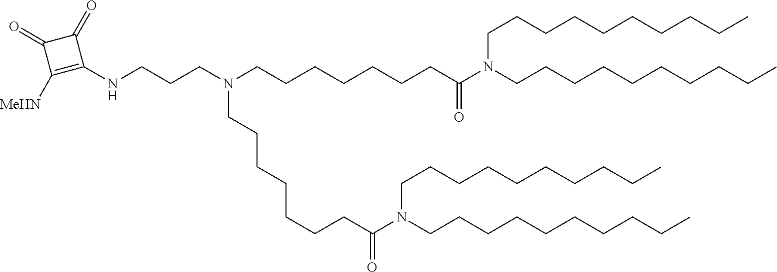 [SMILES: CCCCCCCCCCN(CCCCCCCCCC)C(=O)CCCCCCCN(CCCCCCCC(=O)N(CCCCCCCCCC)CCCCCCCCCC)CCCNc1c(NC)c(=O)c1=O] |
| I-51 | 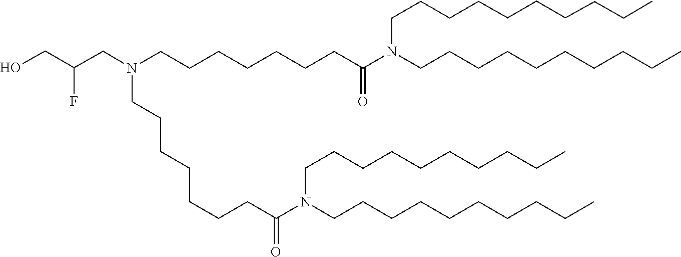 [SMILES: CCCCCCCCCCN(CCCCCCCCCC)C(=O)CCCCCCCN(CCCCCCCC(=O)N(CCCCCCCCCC)CCCCCCCCCC)CC(F)CO] |
| I-52 | 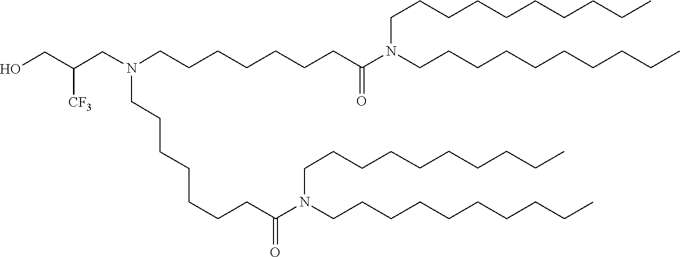 [SMILES: CCCCCCCCCCN(CCCCCCCCCC)C(=O)CCCCCCCN(CCCCCCCC(=O)N(CCCCCCCCCC)CCCCCCCCCC)CC(CO)C(F)(F)F] |
| I-53 | 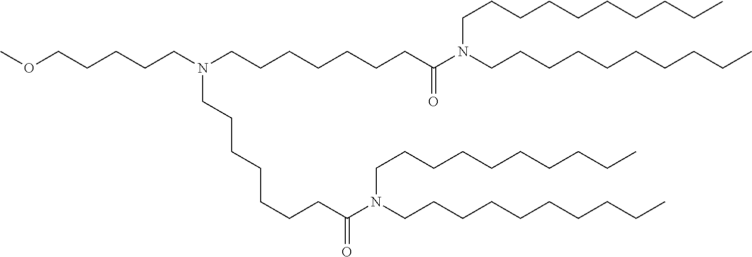 [SMILES: CCCCCCCCCCN(CCCCCCCCCC)C(=O)CCCCCCCN(CCCCCCCC(=O)N(CCCCCCCCCC)CCCCCCCCCC)CCCCCOC] |
| I-54 | 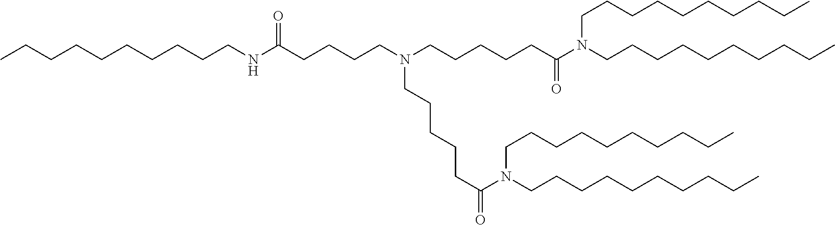 [SMILES: CCCCCCCCCCNC(=O)CCCCN(CCCCCC(=O)N(CCCCCCCCCC)CCCCCCCCCC)CCCCCC(=O)N(CCCCCCCCCC)CCCCCCCCCC] |
| I-55 | 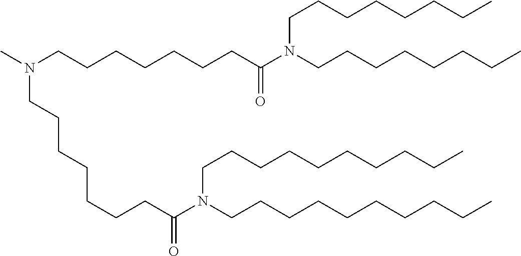 [SMILES: CCCCCCCCCCN(CCCCCCCCCC)C(=O)CCCCCCCN(C)CCCCCCCC(=O)N(CCCCCCCC)CCCCCCCC] |
| I-56 | 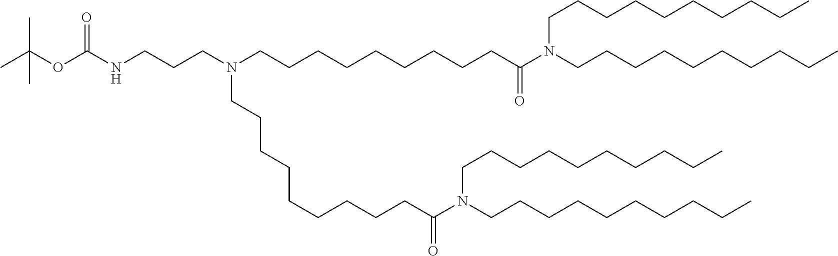 [SMILES: CCCCCCCCCCN(CCCCCCCCCC)C(=O)CCCCCCCCCN(CCCCCCCCCC(=O)N(CCCCCCCCCC)CCCCCCCCCC)CCCNC(=O)OC(C)(C)C] |
| I-57 | 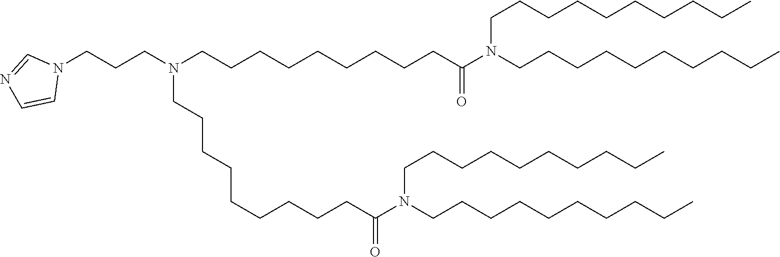 [SMILES: CCCCCCCCCCN(CCCCCCCCCC)C(=O)CCCCCCCCCN(CCCCCCCCCC(=O)N(CCCCCCCCCC)CCCCCCCCCC)CCCn1ccnc1] |
| I-58 | 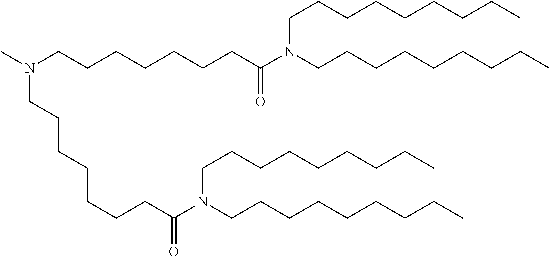 [SMILES: CCCCCCCCCN(CCCCCCCCC)C(=O)CCCCCCCN(C)CCCCCCCC(=O)N(CCCCCCCCC)CCCCCCCCC] |
| I-59 | 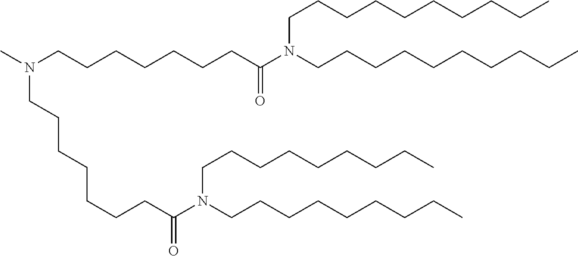 [SMILES: CCCCCCCCCCN(CCCCCCCCCC)C(=O)CCCCCCCN(C)CCCCCCCC(=O)N(CCCCCCCCC)CCCCCCCCC] |
| I-60 | 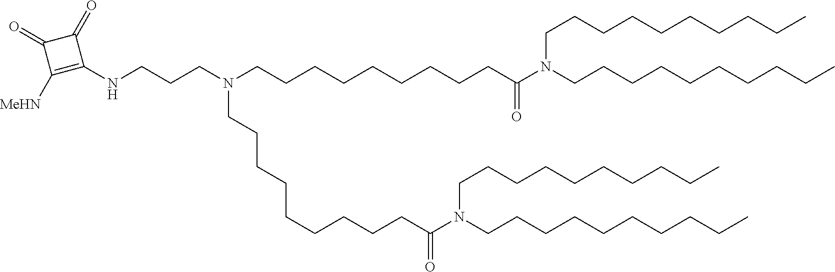 [SMILES: CCCCCCCCCCN(CCCCCCCCCC)C(=O)CCCCCCCCCN(CCCCCCCCCC(=O)N(CCCCCCCCCC)CCCCCCCCCC)CCCNc1c(NC)c(=O)c1=O] |
| I-61 | 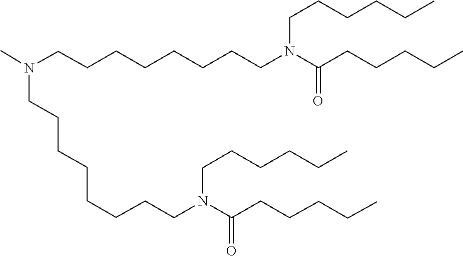 [SMILES: CCCCCCN(CCCCCCCCN(C)CCCCCCCCN(CCCCCC)C(=O)CCCCC)C(=O)CCCCC] |
| I-62 | 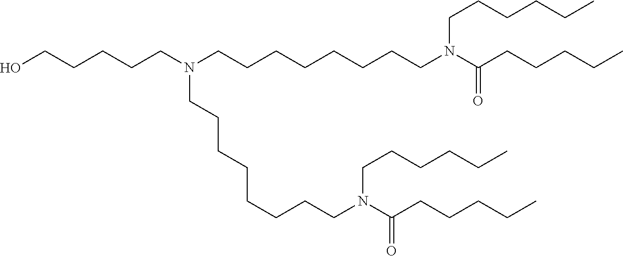 [SMILES: CCCCCCN(CCCCCCCCN(CCCCCO)CCCCCCCCN(CCCCCC)C(=O)CCCCC)C(=O)CCCCC] |
| I-63 | 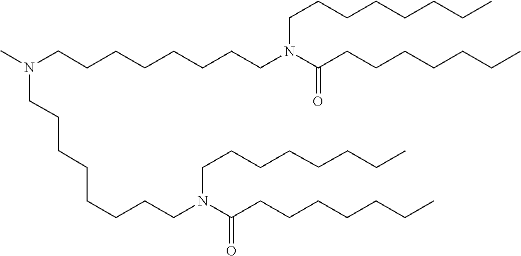 [SMILES: CCCCCCCCN(CCCCCCCCN(C)CCCCCCCCN(CCCCCCCC)C(=O)CCCCCCC)C(=O)CCCCCCC] |
| I-64 | 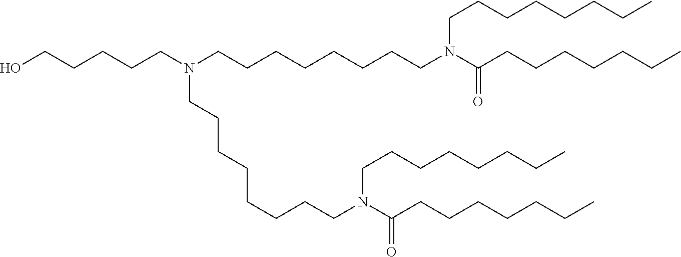 [SMILES: CCCCCCCCN(CCCCCCCCN(CCCCCO)CCCCCCCCN(CCCCCCCC)C(=O)CCCCCCC)C(=O)CCCCCCC] |
| I-65 | 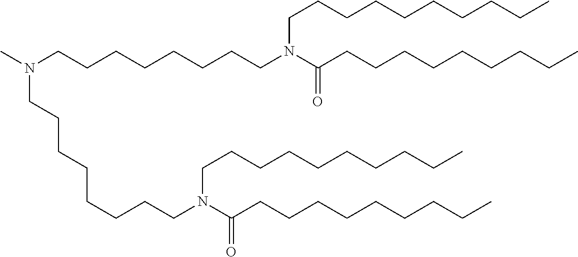 [SMILES: CCCCCCCCCCN(CCCCCCCCN(C)CCCCCCCCN(CCCCCCCCCC)C(=O)CCCCCCCCC)C(=O)CCCCCCCCC] |
| I-66 | 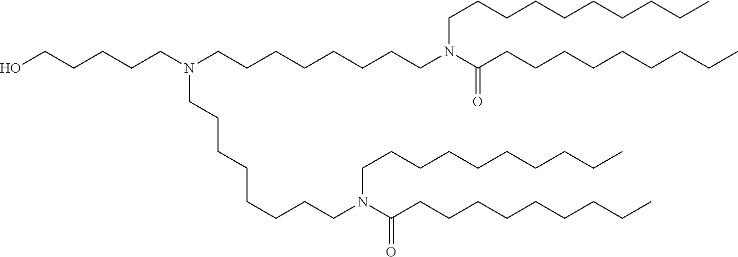 [SMILES: CCCCCCCCCCN(CCCCCCCCN(CCCCCO)CCCCCCCCN(CCCCCCCCCC)C(=O)CCCCCCCCC)C(=O)CCCCCCCCC] |
| I-67 | 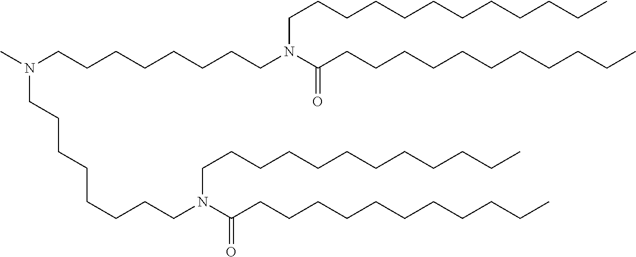 [SMILES: CCCCCCCCCCCCN(CCCCCCCCN(C)CCCCCCCCN(CCCCCCCCCCCC)C(=O)CCCCCCCCCCC)C(=O)CCCCCCCCCCC] |
| I-68 | 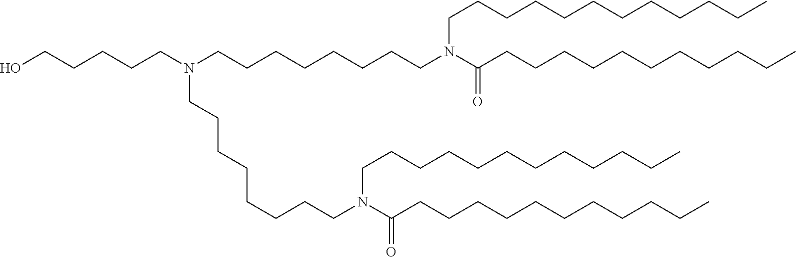 [SMILES: CCCCCCCCCCCCN(CCCCCCCCN(CCCCCO)CCCCCCCCN(CCCCCCCCCCCC)C(=O)CCCCCCCCCCC)C(=O)CCCCCCCCCCC] |
| I-69 | 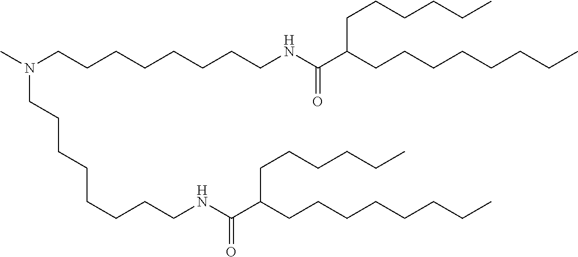 [SMILES: CCCCCCCCC(CCCCCC)C(=O)NCCCCCCCCN(C)CCCCCCCCNC(=O)C(CCCCCC)CCCCCCCC] |
| I-70 | 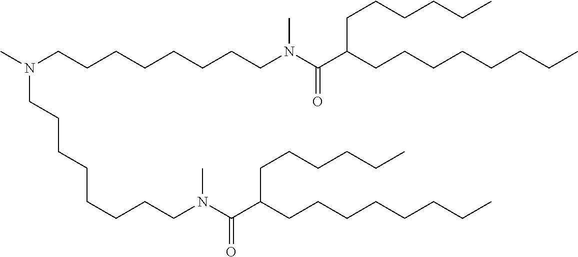 [SMILES: CCCCCCCCC(CCCCCC)C(=O)N(C)CCCCCCCCN(C)CCCCCCCCN(C)C(=O)C(CCCCCC)CCCCCCCC] |
| I-71 | 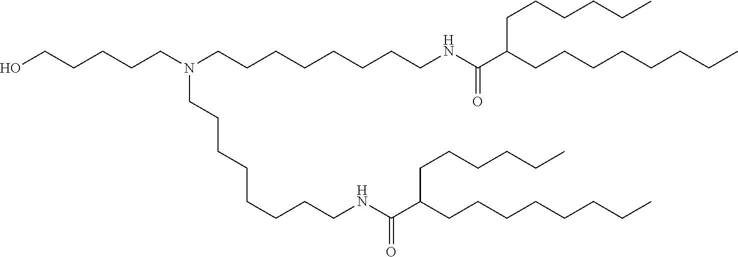 [SMILES: CCCCCCCCC(CCCCCC)C(=O)NCCCCCCCCN(CCCCCO)CCCCCCCCNC(=O)C(CCCCCC)CCCCCCCC] |
| I-72 | 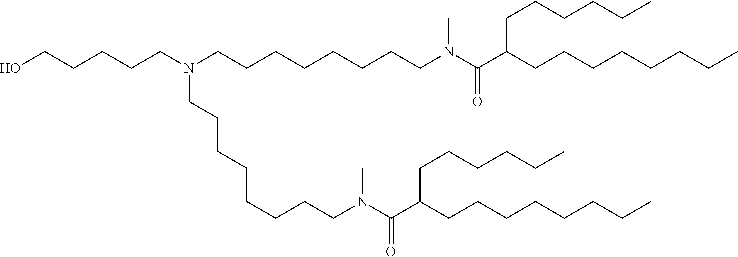 [SMILES: CCCCCCCCC(CCCCCC)C(=O)N(C)CCCCCCCCN(CCCCCO)CCCCCCCCN(C)C(=O)C(CCCCCC)CCCCCCCC] |
| I-73 | 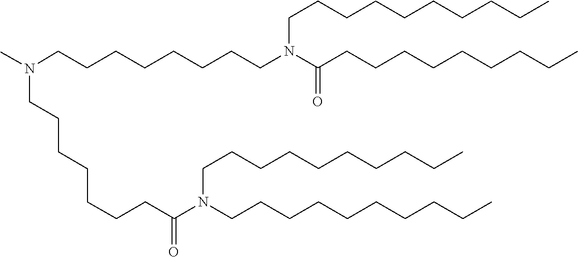 [SMILES: CCCCCCCCCCN(CCCCCCCCN(C)CCCCCCCC(=O)N(CCCCCCCCCC)CCCCCCCCCC)C(=O)CCCCCCCCC] |
| I-74 | 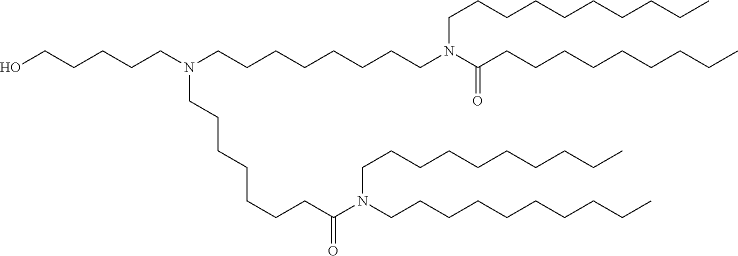 [SMILES: CCCCCCCCCCN(CCCCCCCCN(CCCCCO)CCCCCCCC(=O)N(CCCCCCCCCC)CCCCCCCCCC)C(=O)CCCCCCCCC] |
| I-75 | 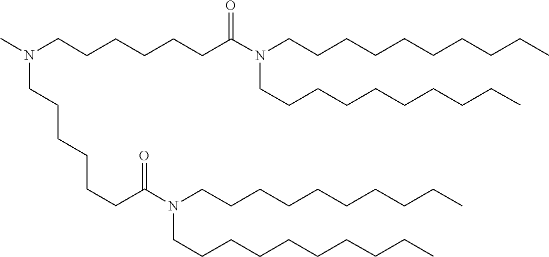 [SMILES: CCCCCCCCCCN(CCCCCCCCCC)C(=O)CCCCCCN(C)CCCCCCC(=O)N(CCCCCCCCCC)CCCCCCCCCC] |
| I-76 | 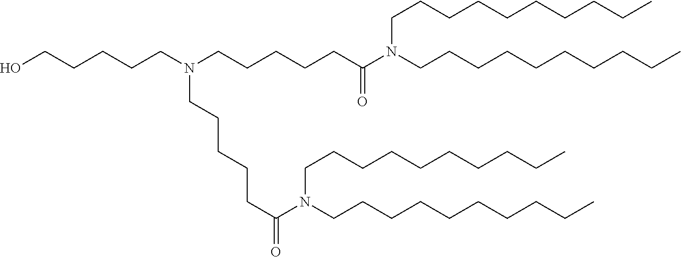 [SMILES: CCCCCCCCCCN(CCCCCCCCCC)C(=O)CCCCCN(CCCCCO)CCCCCC(=O)N(CCCCCCCCCC)CCCCCCCCCC] |
| I-77 | 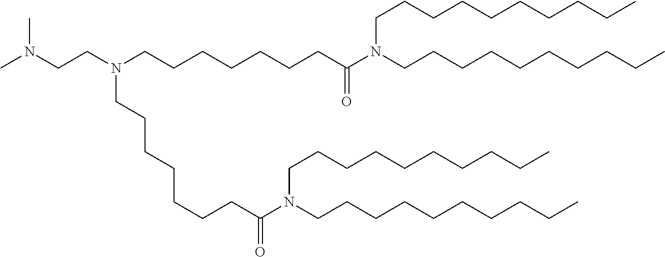 [SMILES: CCCCCCCCCCN(CCCCCCCCCC)C(=O)CCCCCCCN(CCCCCCCC(=O)N(CCCCCCCCCC)CCCCCCCCCC)CCN(C)C] |
| I-78 | 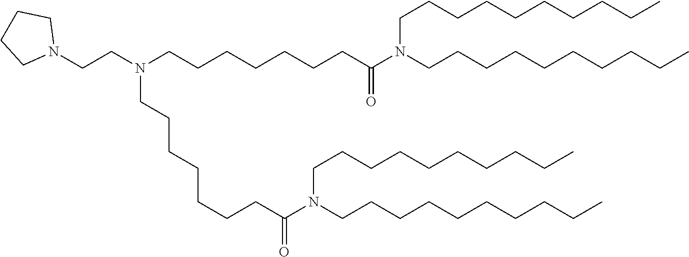 [SMILES: CCCCCCCCCCN(CCCCCCCCCC)C(=O)CCCCCCCN(CCCCCCCC(=O)N(CCCCCCCCCC)CCCCCCCCCC)CCN1CCCC1] |
| I-79 | 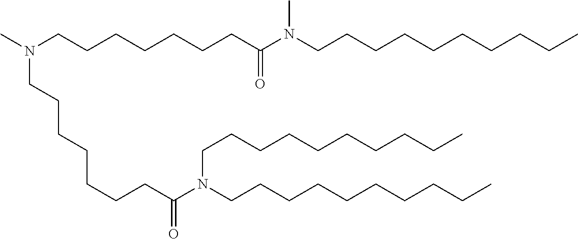 [SMILES: CCCCCCCCCCN(C)C(=O)CCCCCCCN(C)CCCCCCCC(=O)N(CCCCCCCCCC)CCCCCCCCCC] |
| I-80 | 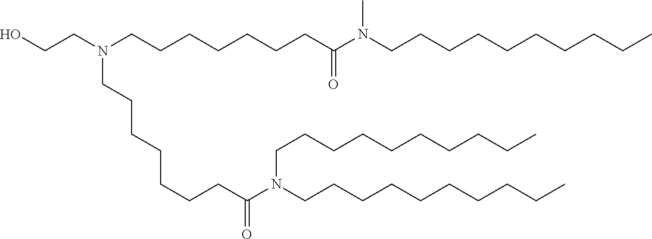 [SMILES: CCCCCCCCCCN(C)C(=O)CCCCCCCN(CCO)CCCCCCCC(=O)N(CCCCCCCCCC)CCCCCCCCCC] |
| I-81 | 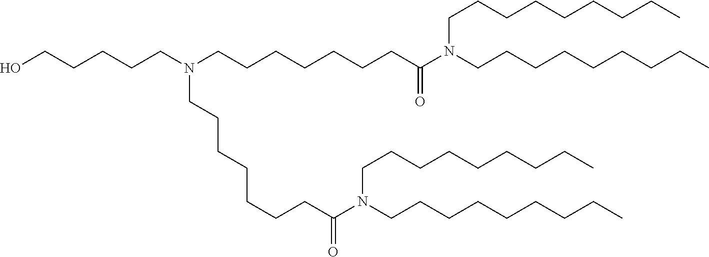 [SMILES: CCCCCCCCCN(CCCCCCCCC)C(=O)CCCCCCCN(CCCCCO)CCCCCCCC(=O)N(CCCCCCCCC)CCCCCCCCC] |
| I-82 | 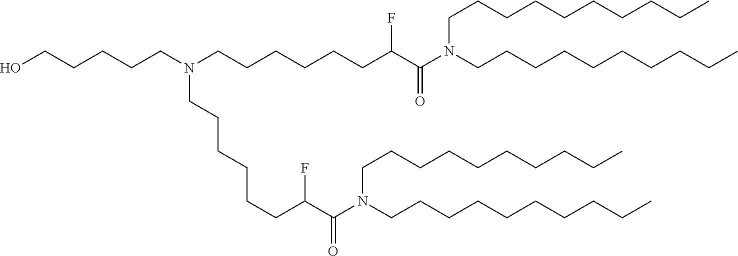 [SMILES: CCCCCCCCCCN(CCCCCCCCCC)C(=O)C(F)CCCCCCN(CCCCCO)CCCCCCC(F)C(=O)N(CCCCCCCCCC)CCCCCCCCCC] |
| I-83 | 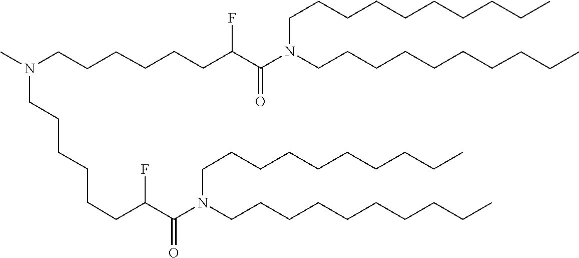 [SMILES: CCCCCCCCCCN(CCCCCCCCCC)C(=O)C(F)CCCCCCN(C)CCCCCCC(F)C(=O)N(CCCCCCCCCC)CCCCCCCCCC] |
| I-84 | 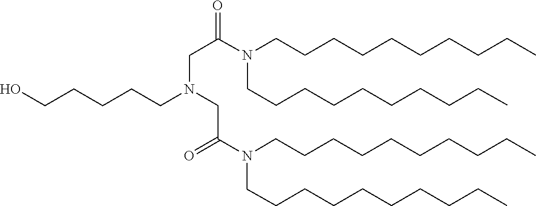 [SMILES: CCCCCCCCCCN(CCCCCCCCCC)C(=O)CN(CCCCCO)CC(=O)N(CCCCCCCCCC)CCCCCCCCCC] |
| I-85 | 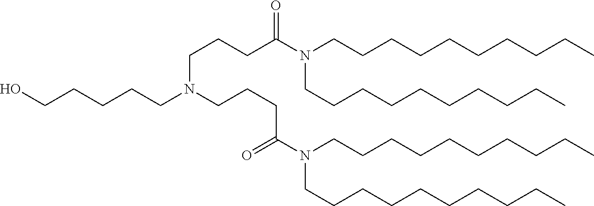 [SMILES: CCCCCCCCCCN(CCCCCCCCCC)C(=O)CCCN(CCCCCO)CCCC(=O)N(CCCCCCCCCC)CCCCCCCCCC] |

The compounds in Table 1 were prepared and tested according to methods known in the art, for example those general methods described herein below.

It is understood that any embodiment of the compounds of structure (I), as set forth above, and any specific substituent and/or variable in the compound structure (I), as set forth above, may be independently combined with other embodiments and/or substituents and/or variables of compounds of structure (I) to form embodiments of the inventions not specifically set forth above. In addition, in the event that a list of substituents and/or variables is listed for any particular R group, L group, G group, or variables a, x, y, or z in a particular embodiment and/or claim, it is understood that each individual substituent and/or variable may be deleted from the particular embodiment and/or claim and that the remaining list of substituents and/or variables will be considered to be within the scope of embodiments of the invention.

It is understood that in the present description, combinations of substituents and/or variables of the depicted formulae are permissible only if such contributions result in stable compounds.

In some embodiments, compositions comprising a compound of structure (I) are provided. In some embodiments, the compositions comprise lipid nanoparticles comprising a compound of structure (I) are provided. The lipid nanoparticles optionally include excipients selected from a neutral lipid, a steroid and a polymer conjugated lipid.

In some embodiments, lipid nanoparticles comprising any one or more of the compounds of structure (I) and a therapeutic agent are provided. For example, in some embodiments, the lipid nanoparticles comprise any of the compounds of structure (I) and a therapeutic agent and one or more excipient selected from neutral lipids, steroids and polymer conjugated lipids. Other pharmaceutically acceptable excipients and/or carriers are also included in various embodiments of the lipid nanoparticles.

In some embodiments, the neutral lipid is selected from DSPC, DPPC, DMPC, DOPC, POPC, DOPE and SM. In some embodiments, the neutral lipid is DSPC. In various embodiments, the molar ratio of the compound to the neutral lipid ranges from about 2:1 to about 8:1.

In various embodiments, the lipid nanoparticles s further comprise a steroid or steroid analogue. In certain embodiments, the steroid or steroid analogue is cholesterol. In some of these embodiments, the molar ratio of the compound to cholesterol ranges from about 5:1 to 1:1 or about 2:1 to 1:1.

In various embodiments, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate. In various embodiments, the molar ratio of the compound to the pegylated lipid ranges from about 100:1 to about 20:1 or about 100:1 to 10:1.

In some embodiments, the composition comprises a pegylated lipid having the following structure (II):

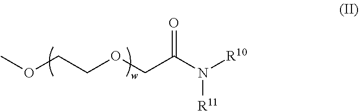

or a pharmaceutically acceptable salt, tautomer or stereoisomer thereof, wherein:
R¹⁰ and R¹¹ are each independently a straight or branched, alkyl, alkenyl or alkynyl containing from 10 to 30 carbon atoms, wherein the alkyl, alkenyl or alkynyl is optionally interrupted by one or more ester bonds; and
w has a mean value ranging from 30 to 60.

In some embodiments, R¹⁰ and R¹¹ are each independently straight alkyl containing from 10 to 30 carbon atoms. In some embodiments, R¹⁰ and R¹¹ are each independently straight alkyl containing 12 to 16 carbon atoms. In some embodiments, w has a mean value ranging from 30 to 60. In some embodiments, w has a mean value ranging 43 to 53. In other embodiments, the average w is about 45. In other different embodiments, the average w is about 49.

Preparation methods for the above lipids, lipid nanoparticles and compositions are described herein below and/or known in the art, for example, in PCT Pub. No. WO 2015/199952, WO 2017/004143 and WO 2017/075531, each of which is incorporated herein by reference in their entireties.

In some embodiments of the foregoing composition, the therapeutic agent comprises a nucleic acid. For example, in some embodiments, the nucleic acid is selected from antisense and messenger RNA.

In other different embodiments, the invention is directed to a method for administering a therapeutic agent to a patient in need thereof, the method comprising preparing or providing any of the foregoing compositions and administering the composition to the patient

For the purposes of administration, the compounds of structure (I) (typically in the form of lipid nanoparticles in combination with a therapeutic agent) may be administered as a raw chemical or may be formulated as pharmaceutical compositions. Pharmaceutical compositions of embodiments of the present invention comprise a compound of structure (I) (e.g., as a component in an LNP) and one or more pharmaceutically acceptable carrier, diluent or excipient. The compound of structure (I) is present in the composition in an amount which is effective to form a lipid nanoparticle and deliver the therapeutic agent, e.g., for treating a particular disease or condition of interest. Appropriate concentrations and dosages can be readily determined by one skilled in the art.

Administration of the compositions and/or LNPs of embodiments of the invention can be carried out via any of the accepted modes of administration of agents for serving similar utilities. The pharmaceutical compositions of embodiments of the invention may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, peritoneal, sublingual, buccal, rectal, vaginal, and intranasal. The term peritoneal as used herein includes subcutaneous injections, intravenous, intramuscular, intradermal, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of structure (I) in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of structure (I), or a pharmaceutically acceptable salt thereof, for treatment of a disease or condition of interest in accordance with the teachings of embodiments of this invention.

A pharmaceutical composition of embodiments of the invention may be in the form of a solid or liquid. In one aspect, the carrier(s) are particulate, so that the compositions are, for example, in tablet or powder form. The carrier(s) may be liquid, with the compositions being, for example, oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds or LNPs, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of embodiments of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose. The peritoneal preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of embodiments of the invention intended for either peritoneal or oral administration should contain an amount of a compound of structure (I) such that a suitable LNP will be obtained.

The pharmaceutical composition of embodiments of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of embodiments of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of embodiments of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of embodiments of the invention in solid or liquid form may include an agent that binds to the compound of structure (I) and thereby assists in the delivery of the compound. Suitable agents that may act in this capacity include a monoclonal or polyclonal antibody, or a protein.

The pharmaceutical composition of embodiments of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of structure (I) may be delivered in single phase, bi-phasic, or tri-phasic systems in order to deliver the active ingredient(s). Delivery of the aerosol includes the necessary container, activators, valves, sub-containers, and the like, which together may form a kit. One skilled in the art, without undue experimentation may determine preferred aerosols.

The pharmaceutical compositions of embodiments of the invention may be prepared by methodology well known in the pharmaceutical art. For example, a pharmaceutical composition intended to be administered by injection can be prepared by combining the lipid nanoparticles of the invention with sterile, distilled water or other carrier so as to form a solution. A surfactant may be added to facilitate the formation of a homogeneous solution or suspension. Surfactants are compounds that non-covalently interact with the compound of structure (I) so as to facilitate dissolution or homogeneous suspension of the compound in the aqueous delivery system.

The compositions of embodiments of the invention, or their pharmaceutically acceptable salts, are administered in a therapeutically effective amount, which will vary depending upon a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the particular disorder or condition; and the subject undergoing therapy.

Compositions of embodiments of the invention may also be administered simultaneously with, prior to, or after administration of one or more other therapeutic agents. Such combination therapy includes administration of a single pharmaceutical dosage formulation of a composition of embodiments of the invention and one or more additional active agents, as well as administration of the composition of the invention and each active agent in its own separate pharmaceutical dosage formulation. For example, a composition of embodiments of the invention and the other active agent can be administered to the patient together in a single oral dosage composition such as a tablet or capsule, or each agent administered in separate oral dosage formulations. Where separate dosage formulations are used, the compounds of structure (I) and one or more additional active agents can be administered at essentially the same time, i.e., concurrently, or at separately staggered times, i.e., sequentially; combination therapy is understood to include all these regimens.

Preparation methods for the above compounds and compositions are described herein below and/or known in the art.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of structure (I) which are pharmacologically active. Such derivatives may therefore be described as “prodrugs”. All prodrugs of compounds of structure (I) are included within the scope of embodiments of the invention.

Furthermore, all compounds of structure (I) which exist in free base or acid form can be converted to their pharmaceutically acceptable salts by treatment with the appropriate inorganic or organic base or acid by methods known to one skilled in the art. Salts of the compounds of structure (I) can be converted to their free base or acid form by standard techniques.

The compounds of structure (I), and lipid nanoparticles comprising the same, can be prepared according to methods known or derivable by one of ordinary skill in the art, for example those methods disclosed in PCT Pub. No. WO 2015/199952, WO 2017/004143 and WO 2017/075531, each of which is incorporated herein by reference in their entireties.

The following General Reaction Schemes illustrate exemplary methods to make compounds of structure (I):

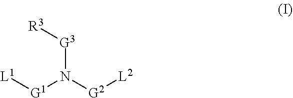

or a pharmaceutically acceptable salt, tautomer or stereoisomer thereof, wherein R³, L¹, L², G¹, G², and G³ are as defined herein. It is understood that one skilled in the art may be able to make these compounds by similar methods or by combining other methods known to one skilled in the art. It is also understood that one skilled in the art would be able to make, in a similar manner as described below, other compounds of structure (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described in this invention.

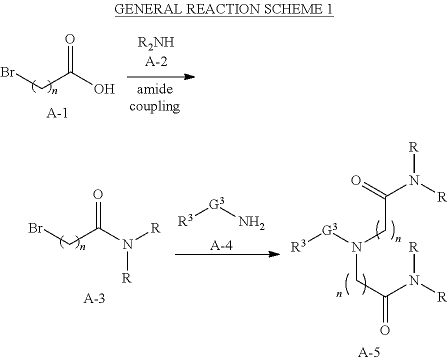

Embodiments of the compound of structure (I) (e.g., compound A-5) can be prepared according to General Reaction Scheme 1 (“Method A”), wherein R, at each occurrence, independently represents R^{b}, R^{c}, R^{e} or R^{f}, and each n is independently an integer from 2 to 12. Referring to General Reaction Scheme 1, compounds of structure A-1 can be purchased from commercial sources or prepared according to methods familiar to one of ordinary skill in the art. A mixture of A-1, A-2 and DMAP is treated with DCC to give the bromide A-3. A mixture of the bromide A-3, a base (e.g., N,N-diisopropylethylamine) and the N,N-dimethyldiamine A-4 is heated at a temperature and time sufficient to produce A-5 after any necessary workup and or purification step.

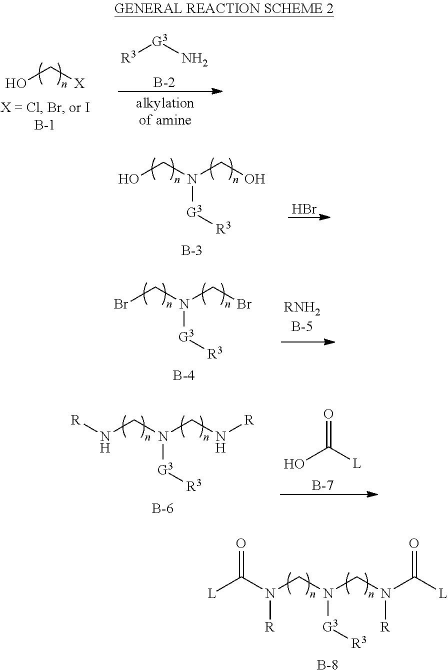

Embodiments of the compound of structure (I) (e.g., compound B-8) can be prepared according to General Reaction Scheme 2 (“Method B”), wherein R, at each occurrence, independently represents R^{a} or R^{d}, L, at each occurrence, independently represents R¹ or R², and each n is independently an integer from 2 to 12. Referring to General Reaction Scheme 2, compounds of structure B-1 can be purchased from commercial sources or prepared according to methods familiar to one of ordinary skill in the art. A nitrogen of B-2 is alkylated with B-1 to afford a diol product B-3, which is then converted into the bromide B-4 with a slow addition of HBr solution followed by a reflux. The resulting bromide B-4 in presence of alkylamine B-5, N,N-diisopropylethylamine, and sodium iodide in a solvent is heated at a temperature and time sufficient to produce B-6 after any necessary workup and or purification step. Into a solution of a carboxylic acid B-7, oxalyl chloride is added dropwise. Then, the solution of B-6 is added to the mixture and stirred, which results in B-8 after any necessary workup and or purification step.

It should be noted that various alternative strategies for preparation of compounds of structure (I) are available to those of ordinary skill in the art. For example, the R³ moiety may include a substituent, such as hydroxyl, and appropriate protecting groups may be required to mask the substituent, or the substituent may be added after R⁵ is added to the remainder of the molecule. The use of protecting groups as needed and other modification to the above General Reaction Scheme 1 will be readily apparent to one of ordinary skill in the art. The following examples are provided for purpose of illustration and not limitation.

### Example 1

### Luciferase mRNA In Vivo Evaluation Using Lipid Nanoparticle Compositions

A lipid of structure (I), DSPC, cholesterol and PEG-lipid are solubilized in ethanol at a molar ratio of 50:10:38.5:1.5 or 47.5:10:40.7:1.8. Lipid nanoparticles (LNP) are prepared at a total lipid to mRNA weight ratio of approximately 10:1 to 40:1. Briefly, the mRNA is diluted to 0.2 mg/mL in 10 to 50 mM citrate buffer, pH 4 or 10 to 25 mM acetate buffer, pH 4. Syringe pumps are used to mix the ethanolic lipid solution with the mRNA aqueous solution at a ratio of about 1:5 to 1:3 (vol/vol) with total flow rates above 15 mL/min. The ethanol is then removed and the external buffer replaced with PBS by dialysis. Finally, the lipid nanoparticles are filtered through a 0.2 m pore sterile filter.

Studies are performed in 6-8 week old female C₅₇BL/6 mice (Charles River) 8-10 week old CD-1 (Harlan) mice (Charles River) according to guidelines established by an institutional animal care committee (ACC) and the Canadian Council on Animal Care (CCAC). Varying doses of mRNA-lipid nanoparticle are systemically administered by tail vein injection and animals euthanized at a specific time point (e.g., 4 hours) post-administration. Liver and spleen are collected in pre-weighed tubes, weights determined, immediately snap frozen in liquid nitrogen and stored at −80° C. until processing for analysis.

For liver, approximately 50 mg is dissected for analyses in a 2 mL FastPrep tubes (MP Biomedicals, Solon OH). ¼″ ceramic sphere (MP Biomedicals) is added to each tube and 500 μL of Glo Lysis Buffer—GLB (Promega, Madison WI) equilibrated to room temperature is added to liver tissue. Liver tissues are homogenized with the FastPrep24 instrument (MP Biomedicals) at 2×6.0 m/s for 15 seconds. Homogenate is incubated at room temperature for 5 minutes prior to a 1:4 dilution in GLB and assessed using SteadyGlo Luciferase assay system (Promega). Specifically, 50 μL of diluted tissue homogenate is reacted with 50 μL of SteadyGlo substrate, shaken for 10 seconds followed by 5 minute incubation and then quantitated using a CentroXS³ LB 960 luminometer (Berthold Technologies, Germany). The amount of protein assayed is determined by using the BCA protein assay kit (Pierce, Rockford, IL). Relative luminescence units (RLU) are then normalized to total μg protein assayed. To convert RLU to ng luciferase a standard curve is generated with QuantiLum Recombinant Luciferase (Promega).

The FLuc mRNA (L-6107) from Trilink Biotechnologies will express a luciferase protein, originally isolated from the firefly, photinus pyralis. FLuc is commonly used in mammalian cell culture to measure both gene expression and cell viability. It emits bioluminescence in the presence of the substrate, luciferin. This capped and polyadenylated mRNA is fully substituted with 5-methylcytidine and pseudouridine.

### Example 2

### Immunoglobulin G (Igg) mRNA In Vivo Evaluation Using Lipid Nanoparticle Compositions

A lipid of structure (I), DSPC, cholesterol and PEG-lipid are solubilized in ethanol at a molar ratio of 50:10:38.5:1.5 or 47.5:10:40.7:1.8. Lipid nanoparticles (LNP) are prepared at a total lipid to mRNA weight ratio of approximately 10:1 to 40:1. Briefly, the mRNA is diluted to 0.2 mg/mL in 10 to 50 mM citrate buffer, pH 4 or 10 to 25 mM acetate buffer, pH 4. Syringe pumps are used to mix the ethanolic lipid solution with the mRNA aqueous solution at a ratio of about 1:5 to 1:3 (vol/vol) with total flow rates above 15 mL/min. The ethanol is then removed and the external buffer replaced with PBS by dialysis. Finally, the lipid nanoparticles are filtered through a 0.2 m pore sterile filter.

Studies are performed in 6-8 week old CD-1/1CR mice (Envigo) according to guidelines established by an institutional animal care committee (ACC) and the Canadian Council on Animal Care (CCAC). Varying doses of mRNA-lipid nanoparticle are systemically administered by tail vein injection and animals euthanized at a specific time point (e.g., 24 hours) post-administration. The whole blood is collected, and the serum subsequentially separated by centrifuging the tubes of the whole blood at 2000×g for 10 minutes at 4° C. and stored at −80° C. until use for analysis.

For immunoglobulin G (IgG) ELISA (Life Diagnostics Human IgG ELISA kit), the serum samples are diluted at 100 to 15000 folds with 1× diluent solution. 100 μL of diluted serum is dispensed into anti-human IgG coated 96-well plate in duplicate alongside human IgG standards and incubated in a plate shaker at 150 rpm at 25° C. for 45 minutes. The wells are washed 5 times with 1× wash solution using a plate washer (400 L/well). 100 μL of HRP conjugate is added into each well and incubated in a plate shaker at the same condition above. The wells are washed 5 times again with 1× wash solution using a plate washer (400 L/well). 100 μL of TMB reagent is added into each well and incubated in a plate shaker at the same condition above. The reaction is stopped by adding 100 μL of Stop solution to each well. The absorbance is read at 450 nm (A450) with a microplate reader. The amount of human IgG in mouse serum is determined by plotting A450 values for the assay standard against human IgG concentration.

### Example 3

### Determination of PK_{A} of Formulated Lipids

As described elsewhere, the pKa of formulated lipids is correlated with the effectiveness of LNPs for delivery of nucleic acids (see Jayaraman et al, Angewandte Chemie, International Edition (2012), 51(34), 8529-8533; Semple et al, Nature Biotechnology 28, 172-176 (2010)). In some embodiments, the preferred range of pKa is ˜5 to ˜7. The pKₐ of each lipid is determined in lipid nanoparticles using an assay based on fluorescence of 2-(p-toluidino)-6-napthalene sulfonic acid (TNS). Lipid nanoparticles comprising compound of structure (I)/DSPC/cholesterol/PEG-lipid (50/10/38.5/1.5 or 47.5:10:40.7:1.8 mol %) in PBS at a concentration of 0.4 mM total lipid are prepared using the in-line process as described in Example 1. TNS is prepared as a 100 μM stock solution in distilled water. Vesicles are diluted to 2 μM lipid in 2 mL of buffered solutions containing 10 mM HEPES, 10 mM MES, 10 mM ammonium acetate, and 130 mM NaCl, where the pH ranged from 2.5 to 11. An aliquot of the TNS solution is added to give a final concentration of 1 μM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis is applied to the fluorescence data and the pKₐ is measured as the pH giving rise to half-maximal fluorescence intensity.

### Example 4

### Determination of Efficacy of Lipid Nanoparticle Formulations Containing Various Cationic Lipids Using an In Vivo Luciferase/IGG mRNA Expression Rodent Model

Representative compounds of the disclosure shown in Table 2 were formulated using the following molar ratio: 50% cationic lipid/10% distearoylphosphatidylcholine (DSPC)/38.5% Cholesterol/1.5% PEG lipid 2-[2-(ω-methoxy(polyethyleneglycol₂₀₀₀)ethoxy]-N,N-ditetradecylacetamide) or 47.5% cationic lipid/10% DSPC/40.7% Cholesterol/1.8% PEG lipid. Relative activity was determined by measuring luciferase expression in the liver 4 hours following administration via tail vein injection as described in Example 1 or by measuring the amount of human IgG in mouse serum as described in example 2. The activity was compared at a dose of 1.0 or 0.5 or 0.3 mg mRNA/kg and expressed as ng luciferase/g liver measured 4 hours after administration, as described in Example 1 or as μg 1 gG/mL serum measured 24 hours after administration, as described in Example 2. Compound numbers in Table 2 refer to the compound numbers of Table 1.

TABLE 2
Novel Cationic Lipids and Associated Activity

| | | Liver Luc | Liver Luc | Serum IgG | Serum IgG |
|---|---|---|---|---|---|
| Cmp. | | @ 0.3 mg/kg | @ 1.0 mg/kg | @ 0.3 mg/kg | @ 1.0 mg/kg |
| No. | pKa | (ng/g) | (ng/g) | (ug/mL) | (ug/mL) |
| I-3 | 6.87 | 227 ± 138* | — | — | — |
| | | * Determined at | | | |
| | | 0.5 mg/kg | | | |
| I-6 | 6.19 | 20084 ± 5967 | 78367 ± 15904 | — | — |
| I-7 | 6.43 | 16595 ± 836 | 81021 ± 24105 | — | — |
| I-8 | 6.02 | 8743 ± 2153 | 37220 ± 15377 | — | — |
| I-9 | 6.83 | 4337 ± 1394 | 21023 ± 12693 | — | — |
| I-10 | 6.77 | 10779 ± 2473 | 22622 ± 8189 | — | — |
| I-11 | 6.67 | — | — | 8.22 ± 0.84 | 47.68 ± 5.26 |
| I-14 | 6.59 | 2738 ± 728 | 5735 ± 2364 | — | — |
| I-18 | 6.02 | 7115 ± 2087 | 14879 ± 1595 | — | — |
| I-20 | 6.27 | 879 ± 365* | — | — | — |
| | | * Determined at | | | |
| | | 0.5 mg/kg | | | |
| I-21 | 6.54 | 29492 ± 4887 | 43216 ± 25636 | 48.78 ± 6.88 | 220.38 ± 62.57 |
| I-22 | 6.47 | 12829 ± 3326 | 38933 ± 23162 | 31.00 ± 9.33 | 99.12 ± 18.03 |
| I-23 | 6.48 | 15876 ± 7588 | 34202 ± 19178 | 43.34 ± 7.70 | 103.91 ± 31.29 |
| I-24 | 6.24 | 9106 ± 1760 | 38913 ± 14318 | 27.46 ± 9.22 | 112.31 ± 14.51 |
| I-25 | 6.28 | 20276 ± 3044 | 64656 ± 24090 | — | — |
| I-27 | 6.56 | 21 ± 6* | — | — | — |
| | | * Determined at | | | |
| | | 0.5 mg/kg | | | |
| I-28 | 6.22 | 6602 ± 2609 | 13298 ± 4040 | — | — |
| I-30 | 6.16 | 6567 ± 1534 | 19625 ± 2173 | — | — |
| I-37 | 6.39 | — | — | 1.75 ± 0.58 | 10.05 ± 2.32 |
| I-38 | 6.48 | — | — | 4.71 ± 0.46 | 45.87 ± 2.33 |
| I-40 | 6.17 | — | — | 16.72 ± 5.38 | 101.96 ± 4.12 |
| I-41 | 6.60 | — | — | 4.78 ± 1.65 | — |
| I-42 | 6.66 | — | — | 3.13 ± 0.27 | 12.42 ± 3.81 |
| I-43 | 6.02 | — | — | 2.45 ± 0.66 | 9.97 ± 1.17 |
| I-44 | 6.69 | — | — | 0.59 ± 0.11 | 1.86 ± 0.27 |
| I-45 | 5.96 | — | — | 3.03 ± 0.66 | 13.00 ± 2.05 |
| I-46 | 5.91 | 10201 ± 2440 | 52937 ± 8101 | — | — |
| I-48 | 6.63 | — | — | 1.02 ± 0.20 | 3.40 ± 0.87 |
| I-50 | 6.73 | — | — | 1.32 ± 0.28 | 11.05 ± 2.19 |
| I-55 | 6.22 | 8244 ± 1599 | 30106 ± 13756 | — | — |
| I-57 | 6.24 | 476 ± 134 | 1456 ± 518 | — | — |
| I-58 | 6.19 | 18664 ± 5438 | 73794 ± 14083 | — | — |
| I-59 | 6.27 | 10786 ± 1924 | 43489 ± 5698 | — | — |
| I-60 | 6.38 | 2176 ± 162 | 9178 ± 2483 | — | — |
| I-65 | 6.33 | 3172 ± 658 | 24740 ± 6297 | — | — |
| I-66 | 6.49 | — | — | 32.06 ± 5.07 | 253.37 ± 52.48 |
| I-67 | 6.18 | — | — | 7.19 ± 1.41 | 62.94 ± 13.88 |
| I-68 | 6.41 | — | — | 12.30 ± 2.41 | 121.93 ± 34.85 |
| I-70 | 6.17 | 7516 ± 1487 | 33238 ± 9902 | — | — |
| I-73 | 6.32 | — | — | 27.72 ± 6.23 | 213.39 ± 18.85 |
| I-74 | 6.53 | — | — | 50.16 ± 6.58 | 292.44 ± 40.55 |
| I-75 | 6.48 | — | — | 84.87 ± 12.19 | 343.06 ± 42.39 |
| I-77 | 6.71 | — | — | 17.45 ± 2.05 | — |
| I-82 | 6.47 | — | — | 24.36 ± 4.39 | 239.26 ± 39.80 |
| I-83 | 6.12 | — | — | 12.94 ± 2.03 | 142.26 ± 26.31 |
| I-85 | 6.81 | — | — | 10.74 ± 2.79 | — |

### Example 5

### 6,6′-(Methylazanediyl)Bis(N,N-Dioctylhexanamide) (Compound I-1)

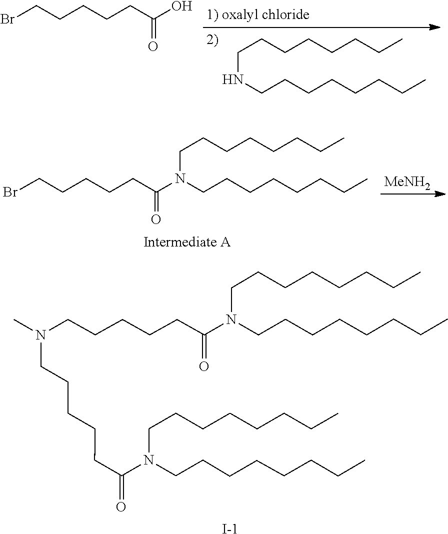

### Synthesis of 6-bromo-N,N-dioctylhexanamide (Intermediate A)

To a solution of 6-bromohexanoic acid (2.93 g, 15 mmol) in DCM (25 mL) and DMF (0.1 mL) was added oxalyl chloride (45 mmol, 5.7 g, 3.93 mL) at RT under Ar. The resulting mixture was stirred at RT overnight. Next day the mixture was concentrated. The residue was dissolved in 20 mL of DCM and added slowly to a solution of dioctylamine (1.1 eq, 16.5 mmol, 3.98 g, 4.98 mL) and triethylamine (90 mmol, 9.09 g, 12.5 mL) and DMAP (10 mg) in DCM (20 mL) at RT. When the addition was complete, the mixture stirred at for 2.5 h and then concentrated. The residue was taken up in diluted HCl and filtered. The filtrate was extracted with DCM three times. Evaporation of the extracts after washing with brine and dried over NaSO4 gave a yellow oil. The oil was purified by chromatography (hexane and ethyl acetate (1:0 to 4:1). The desired product was obtained as slightly yellow oil, 5.09 g, 12.2 mmol, 81%.

### Synthesis of I-1

To a mixture of 6-bromo-N,N-dioctylhexanamide (1.19 mmol, 500 mg), anhydrous acetonitrile (15 mL) and N,N-diisopropylethylamine (1.89 mmol, 244 mg, 0.33 mL) was added methylamine (0.32 mL, 0.63 mmol, 2M in THF). The mixture was heated at 80° C. for 16 h in a sealed pressure flask. The reaction mixture was concentrated. The crude product was purified by flash dry column chromatography on silica gel (hexane/EtOAc/Et₃N, 95:5:0 to 80:20:1). The desired product was obtained as a colorless oil (183 mg, 0.26 mmol, 44%). ¹HNMR (400 MHz, CDCl₃ at 7.27 ppm) δ: 3.32-3.25 (m, 4H), 3.23-3.16 (m, 4H), 2.34-2.26 (m, 8H), 2.20 (s, 3H), 1.66 (quintet, 7.6 Hz, 4H), 1.57-1.44 (m, 12H), 1.38-1.19 (m, 44H), 0.92-0.86 (m, 12H). ESI-MS: MW for C₄₅H₉₁N₃O₂ [M+H]⁺ Calc. 706.7; Found 706.8.

### Example 6

### 6,6′-(Octylazanediyl)Bis(N,N-Dioctylhexanamide) (Compound I-4)

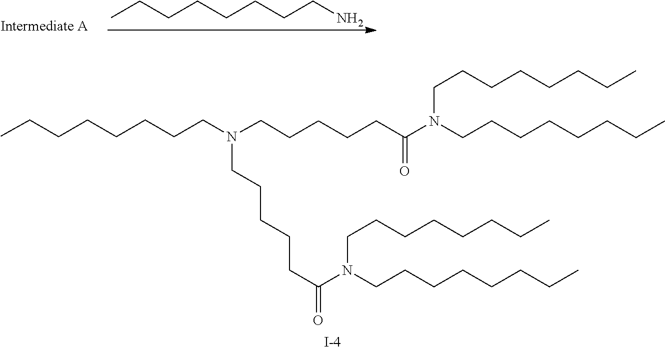

### Synthesis of I-4

To a mixture of 6-bromo-N,N-dioctylhexanamide (1.6 eq, 1.19 mmol, 500 mg), anhydrous acetonitrile (15 mL) and N,N-diisopropylethylamine (1.89 mmol, 244 mg, 0.33 mL) was added octylamine (0.123 mL, 0.74 mmol). The mixture was heated at 80° C. for 16 h in a sealed pressure flask. The reaction mixture was concentrated. The crude product was purified by flash dry column chromatography on silica gel (hexane/EtOAc/Et₃N, 95:5:0 to 80:20:1) and further purified using 0 to 5% MeOH in chloroform as elution solvent mixture. The desired product was obtained as a colorless oil (173 mg, 0.22 mmol, 36%). ¹HNMR (400 MHz, CDCl3) δ: 3.33-3.25 (m, 4H), 3.23-3.16 (m, 4H), 3.08-2.86 (br. 3H), 2.5-2.32 (br, 3H), 2.29 (t, 7.6 Hz, 4H), 1.85-1.70 (br, 2H), 1.67 (quintet, 7 Hz, 4H), 1.57-1.38 (m, 12H), 1.38-1.17 (m, 54H), 0.92-0.86 (m, 15H). ESI-MS: MW for C₅₂H₁₀₅N₃O₂ [M+H]⁺ Calc. 804.8; Found 805.0.

### Example 7

### 6,6′-(Hexylazanediyl)Bis(N,N-Dioctylhexanamide) (Compound I-5)

Compound I-5 was prepared according to the general procedures of example 6 to yield the desired product as a colorless oil (155 mg, 0.20 mmol, 34%). ¹HNMR (400 MHz, CDCl₃ at 7.27 ppm) δ: 3.33-3.25 (m, 4H), 3.24-3.15 (m, 4H), 3.10-2.80 (br. 2H), 2.58-2.32 (br, 4H), 2.29 (t, 7.4 Hz, 4H), 1.88-1.69 (br, 2H), 1.66 (quintet, 7.4 Hz, 4H), 1.57-1.39 (m, 12H), 1.39-1.17 (m, 50H), 0.92-0.86 (m, 15H).

### Example 8

### 8,8′-(Methylazanediyl)Bis(N,N-Dioctyloctanamide) (Compound I-7)

Compound I-7 was prepared according to the general procedures of example 5 to yield the desired product as a slightly yellow oil (230 mg, 0.30 mmol, 59%). ¹HNMR (400 MHz, CDCl₃ at 7.27 ppm) δ: 3.32-3.25 (m, 4H), 3.23-3.15 (m, 4H), 2.31-2.25 (m, 8H), 2.20 (s, 3H), 1.64 (quintet, 7.3 Hz, 4H), 1.57-1.41 (m, 12H), 1.38-1.19 (m, 52H), 0.92-0.86 (m, 12H). ESI-MS: MW for C₄₉H₉₉N₃O₂ [M+H]⁺ Calc. 762.8; Found 762.9.

### Example 9

### 10,10′-(Methylazanediyl)Bis(N,N-Dioctyldecanamide) (Compound I-8)

Compound I-8 was prepared according to the general procedures of example 5 to yield the desired product as a colorless oil (205 mg, 0.25 mmol, 64%). ¹HNMR (400 MHz, CDCl₃ at 7.27 ppm) δ: 3.33-3.25 (m, 4H), 3.23-3.16 (m, 4H), 2.31-2.25 (q-like, 8H), 2.20 (s, 3H), 1.63 (quintet, 7.3 Hz, 4H), 1.57-1.41 (m, 12H), 1.38-1.19 (m, 60H), 0.92-0.86 (m, 12H). ESI-MS: MW for C₅₃H₁₀₇N₃O₂ [M+H]⁺ Calc. 818.8; Found 819.0.

### Example 10

### 8,8′-(Methylazanediyl)Bis(N,N-Didecyloctanamide) (Compound I-6)

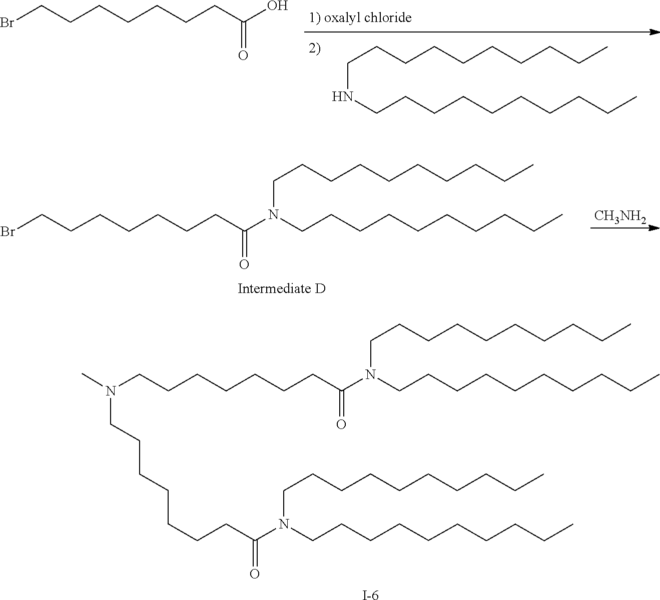

### Synthesis of 8-bromo-N,N-didecyloctanamide (Intermediate D)

To a solution of 8-bromooctanoic acid (1 eq. 10.78 mmol, 2.41 g) in DCM (20 mL) and DMF (d 0.944; 0.1 mL) was added oxalyl chloride (2.5 eq, 27 mmol, 3.42 g, 2.35 mL) at RT under Ar. The resulting mixture was stirred at RT overnight. Next, the mixture was concentrated under reduced pressure. The residue was dissolved in 15 mL of DCM and added slowly to a solution of didecylamine (1.1 eq, 3.53 g, 11.86 mmol) and triethylamine (53.9 mmol, 7.5 mL) and DMAP (10 mg) in DCM (20 mL) at RT. When the addition was complete, the mixture stirred at RT overnight and then concentrated. The residue was taken up in hexane (100 mL) and was loaded on a silica gel column under reduced pressure. Then column was washed with a mixture of hexane and ethyl acetate (100:0 to 90:10) under reduced pressure. The desired product was obtained as yellow oil, 4.81 g, 9.6 mmol, 89%.

### Synthesis of I-6

A mixture of 8-bromo-N,N-didecyloctanamide (0.8 mmol, 400 mg), CH₃CN (15 mL) and DIPEA (0.26 mL) and methylamine (33 wt % in EtOH, 0.062 mL, ca 0.5 mmol) was sealed in a pressure flask and heated at 74° C. overnight. The reaction mixture was concentrated. The crude product was purified by flash dry column chromatography on silica gel (hexane/EtOAc/Et₃N, 95:5:0 to 80:20:1) and further purified using 0 to 5% MeOH in chloroform with a trace of Et₃N as elution solvent mixture. The desired product was obtained as a colorless oil (167 mg, 0.19 mmol, 48%). ¹HNMR (400 MHz, CDCl₃ at 7.26 ppm) δ: 3.32-3.24 (m, 4H), 3.23-3.15 (m, 4H), 2.30-2.24 (m, 8H), 2.18 (s, 3H), 1.63 (quintet, 7.1 Hz, 4H), 1.57-1.38 (m, 12H), 1.38-1.19 (m, 68H), 0.92-0.86 (m, 12H). ESI-MS: MW for C₅₇H₁₁₅N₃O₂ [M+H]⁺ Calc. 874.9; Found 875.1.

### Example 11

### 6,6′-(Methylazanediyl)Bis(N,N-Didecylhexanamide) (Compound I-10)

Compound I-10 was prepared according to the general procedures of example 5 to yield the desired product as a colorless oil (249 mg, 0.30 mmol, 58%). ¹HNMR (400 MHz, CDCl₃ at 7.26 ppm) δ: 3.32-3.24 (m, 4H), 3.22-3.15 (m, 4H), 2.33-2.25 (m, 8H), 2.19 (s, 3H), 1.65 (quintet, 7.6 Hz, 4H), 1.57-1.41 (m, 12H), 1.38-1.08 (m, 60H), 0.91-0.85 (m, 12H). ESI-MS: MW for C₅₃H₁₀₇N₃O₂ [M+H]⁺ Calc. 818.8; Found 819.0.

### Example 12

### 6,6′-(Methylazanediyl)Bis(N,N-Didodecylhexanamide) (Compound I-11)

Compound I-11 was prepared according to the general procedures of example 5 to yield the desired product as a colorless oil (313 mg, 0.34 mmol, 68%). ¹HNMR (400 MHz, CDCl₃) δ: 3.32-3.24 (m, 4H), 3.22-3.15 (m, 4H), 2.33-2.25 (m, 8H), 2.19 (s, 3H), 1.65 (quintet, 7.6 Hz, 4H), 1.57-1.43 (m, 12H), 1.37-1.10 (m, 76H), 0.91-0.85 (m, 12H). ESI-MS: MW for C₆₁H₁₂₃N₃O₂ [M+H]⁺ Calc. 931.0; Found 931.2.

### Example 13

### 6,6′-((2-Hydroxyethyl)Azanediyl)Bis(N,N-Dioctylhexanamide) (Compound I-3)

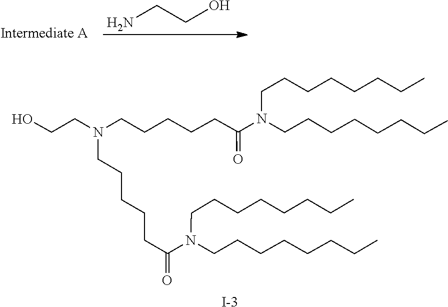

### Synthesis of I-3

A mixture of 2-aminoethanol (1.25 mmol, 77 mg) in 15 ml of anhydrous THF, 6-bromo-N,N-dioctylhexanamide (Intermediate A, 1.9 eq, 1 g, 2.39 mmol), potassium carbonate (1.9 eq, 330 mg, 2.39 mmol), cesium carbonate (0.3 eq, 234 mg, 0.72 mmol) and sodium iodide (3 mg) in a pressure flask was heated for 6 days at 77° C. (oil bath). The reaction mixture was concentrated. The crude product was purified by flash dry column chromatography on silica gel (hexane/EtOAc/Et₃N, 95:5:0 to 80:20:1). The desired product was obtained as a colorless oil (233 mg, 0.31 mmol, 26%). ¹HNMR (400 MHz, CDCl₃) δ: 3.52 (t, 5.3 Hz, 2H), 3.23-3.25 (m, 4H), 3.24-3.15 (m, 4H), 3.10-2.96 (br. 1H), 2.57 (t, 5.3 Hz, 2H), 2.50-2.41 (m, 4H), 2.28 (t, 7.5 Hz, 4H), 1.65 (quintet, 7.6 Hz, 4H), 1.60-1.42 (m, 12H, overlap with water), 1.38-1.19 (m, 44H), 0.92-0.86 (m, 12H). ESI-MS: MW for C₄₆H₉₃N₃O₃ [M+H]⁺ Calc. 736.7; Found 736.8.

### Example 14

### 6,6′-((6-Hydroxyhexyl)Azanediyl)Bis(N,N-Dioctylhexanamide) (Compound I-16)

Compound I-16 was prepared according to the general procedures of example 13 to yield the desired product as a colorless oil (63 mg). ¹HNMR (400 MHz, CDCl₃) δ: 3.68-3.62 (m, 2H), 3.32-3.24 (m, 4H), 3.23-3.15 (m, 4H), 3.07-2.91 (br, 6H), 2.31 (t, 7.5 Hz, 4H), 1.93-1.73 (br., 6H), 1.68 (quintet, 7.6 Hz, 4H), 1.63-1.46 (m, 10H, overlap with water), 1.46-1.35 (m, 8H), 1.35-1.10 (m, 40H), 0.92-0.86 (m, 12H). The product exists partially in its HCl salt form. ESI-MS: MW for C₅₀H₁₀₁N₃O₃[M+H]⁺ Calc. 792.8; Found 792.9.

### Example 15

### 6,6′-((2-Hydroxyethyl)Azanediyl)Bis(N,N-Didecylhexanamide) (Compound I-19)

Compound I-19 was prepared according to the general procedures of example 13 to yield the desired product as a slightly yellow oil (154 mg, 0.18 mmol, 35%). ¹HNMR (400 MHz, CDCl₃) δ: 3.51 (t, 5.2, 2H), 3.32-3.24 (m, 4H), 3.23-3.15 (m, 4H), 3.08-2.92 (br, 1H), 2.56 (t, 5.3 Hz, 2H), 2.45 (t, 7.4 Hz, 4H), 2.27 (t, 7.5 Hz, 4H), 1.64 (quintet, 7.5 Hz, 4H), 1.59-1.40 (m, 12H, overlapped with water peak), 1.36-1.10 (m, 60H), 0.91-0.86 (m, 12H). ESI-MS: MW for C₅₄H₁₀₉N₃O₃ [M+H]⁺ Calc. 848.9; Found 849.0.

### Example 16

### 8,8′-((6-Hydroxyhexyl)Azanediyl)Bis(N,N-Dioctyloctanamide) (Compound I-17)

Compound I-17 was prepared according to the general procedures of example 13 to yield the desired product as a slightly yellow oil (546 mg, 0.64 mmol, 54%). ¹HNMR (400 MHz, CDCl₃) δ: 3.65 (t, 6.4 Hz, 2H), 3.33-3.25 m, 4H), 3.23-3.15 (m, 4H), 2.40-2.34 (m, 6H), 2.28 (t, 7.5 Hz, 4H), 1.75-1.59 (m, 6H), 1.58-1.46 (m, 8H), 1.46-1.36 (m, 8H), 1.36-1.10 (m, 54H), 0.92-0.86 (m, 12H). ESI-MS: MW for C₅₄H₁₀₉N₃O₃ [M+H]⁺ Calc. 848.9; Found 849.0.

### Example 17

### 10,10′-((6-Hydroxyhexyl)Azanediyl)Bis(N,N-Dioctyldecanamide) (Compound I-18)

Compound I-18 was prepared according to the general procedures of example 13 to yield the desired product as a slightly yellow oil (251 mg, 0.28 mmol, 52%). ¹HNMR (400 MHz, CDCl₃) δ: 3.67-3.63 (m, 2H), 3.33-3.25 m, 4H), 3.24-3.16 (m, 4H), 2.41-2.35 (m, 6H), 2.28 (t, 7.5 Hz, 4H), 1.68-1.59 (m, 6H), 1.59-1.47 (m, 8H, overlapped with water peak), 1.47-1.37 (m, 8H), 1.37-1.10 (m, 62H), 0.92-0.86 (m, 12H).

### Example 18

### 10,10′-((2-Hydroxyethyl)Azanediyl)Bis(N,N-Didecyldecanamide) (Compound I-20)

Compound I-20 was prepared according to the general procedures of example 13 to yield the desired product as a colorless oil (248 mg, 0.26 mmol, 49%). ¹HNMR (400 MHz, CDCl₃) δ: 3.51 (t, 5.4, 2H), 3.32-3.24 (m, 4H), 3.22-3.15 (m, 4H), 3.08-2.92 (br, 1H), 2.56 (t, 5.4 Hz, 2H), 2.43 (t, 7.4 Hz, 4H), 2.27 (t, 7.5 Hz, 4H), 1.64 (quintet, 7.5 Hz, 4H), 1.59-1.36 (m, 12H, overlapped with water peak), 1.36-1.10 (m, 76H), 0.91-0.86 (m, 12H). ESI-MS: MW for C₆₂H₁₂₅N₃O₃ [M+H]⁺ Calc. 961.0; Found 961.1.

### Example 19

### 8,8′-((5-Hydroxypentyl)Azanediyl)Bis(N,N-Didecyloctanamide) (Compound I-21)

Compound I-21 was prepared according to the general procedures of example 13 to yield the desired product as a colorless oil (166 mg, 0.18 mmol, 35%). ¹HNMR (400 MHz, CDCl₃ at 7.26 ppm) δ: 3.64 (t, 6.5 Hz, 2H), 3.32-3.24 (m, 4H), 3.22-3.15 (m, 4H), 2.41-2.34 (m, 6H), 2.27 (t, 7.5 Hz, 4H), 2.05 (br. s, 1H), 1.67-1.57 (m, 6H), 1.54-1.36 (m, 16H), 1.36-1.10 (m, 68H), 0.90-0.85 (m, 12H). ESI-MS: MW for C₆₁H₁₂₃N₃O₃ [M+H]⁺ Calc. 947.0; Found 947.2.

### Example 20

### 8,8′-((4-Hydroxybutyl)Azanediyl)Bis(N,N-Didecyloctanamide) (Compound I-23)

Compound I-23 was prepared according to the general procedures of example 13 to yield the desired product as a colorless oil (209 mg, 0.22 mmol, 45%). ¹HNMR (400 MHz, CDCl₃ at 7.26 ppm) δ: 6.64 (br. s, 1H), 3.57-3.50 (m, 2H), 3.32-3.24 (m, 4H), 3.23-3.15 (m, 4H), 2.44-2.39 (m, 6H), 2.26 (t, 7.5 Hz, 4H), 1.67-1.57 (m, 8H), 1.54-1.37 (m, 12H), 1.36-1.10 (m, 68H), 0.90-0.85 (m, 12H). ESI-MS: MW for C₆₀H₁₂₁N₃O₃ [M+H]⁺ Calc. 932.9; Found 933.1.

### Example 21

### 8,8′-((6-Hydroxyhexyl)Azanediyl)Bis(N,N-Didecyloctanamide) (Compound I-24)

Compound I-24 was prepared according to the general procedures of example 13 to yield the desired product as a colorless oil (229 mg, 0.24 mmol, 48%). ¹HNMR (400 MHz, CDCl₃, at 7.26 ppm) δ: 3.67-3.59 (m, 2H), 3.32-3.24 (m, 4H), 3.23-3.15 (m, 4H), 2.39-2.33 (m, 6H), 2.26 (t, 7.5 Hz, 4H), 1.73-1.68 (br. 1H), 1.68-1.38 (m, est. 20H), 1.38-1.10 (m, 72H), 0.90-0.85 (m, 12H). ESI-MS: MW for C₆₂H₁₂₅N₃O₃ [M+H]⁺ Calc. 961.0; Found 961.1.

### Example 22

### 8,8′-((2-Hydroxyethyl)Azanediyl)Bis(N,N-Didecyloctanamide) (Compound I-22)

Compound I-22 was prepared according to the general procedures of example 13 to yield the desired product as a colorless oil (165 mg, 0.18 mmol, 35%). ¹HNMR (400 MHz, CDCl₃) δ: 3.51 (t, 5.4, 2H), 3.32-3.24 (m, 4H), 3.23-3.15 (m, 4H), 3.07-2.98 (br, 1H), 2.56 (t, 5.4 Hz, 2H), 2.42 (t, 7.4 Hz, 4H), 2.27 (t, 7.5 Hz, 4H), 1.68-1.59 (m, 4H), 1.59-1.36 (m, 12H, overlapped with water peak), 1.36-1.10 (m, 68H), 0.91-0.86 (m, 12H). ESI-MS: MW for C₅₈H₁₁₇N₃O₃ [M+H]⁺ Calc. 904.9; Found 905.1.

### Example 23

### 10,10′-((4-Hydroxybutyl)Azanediyl)Bis(N,N-Didecyldecanamide) (Compound I-25)

Compound I-25 was prepared according to the general procedures of example 13 to yield the desired product as a colorless oil (202 mg, 0.21 mmol, 42%). ¹HNMR (400 MHz, CDCl₃, at 7.26 ppm) δ: 6.66 (br. s, 1H), 3.58-3.50 (m, 2H), 3.32-3.24 (m, 4H), 3.23-3.15 (m, 4H), 2.44-2.39 (m, 6H), 2.26 (t, 7.5 Hz, 4H), 1.70-1.58 (m, 8H), 1.55-1.38 (m, 12H), 1.37-1.14 (m, 76H), 0.90-0.85 (m, 12H). ESI-MS: MW for C₆₄H₁₂₉N₃O₃ [M+H]⁺ Calc. 989.0; Found 989.1.

### Example 24

### N,N′-((Methylazanediyl)Bis(Hexane-6,1-Diyl))Bis(N,2-Dihexyldecanamide) (Compound I-15)

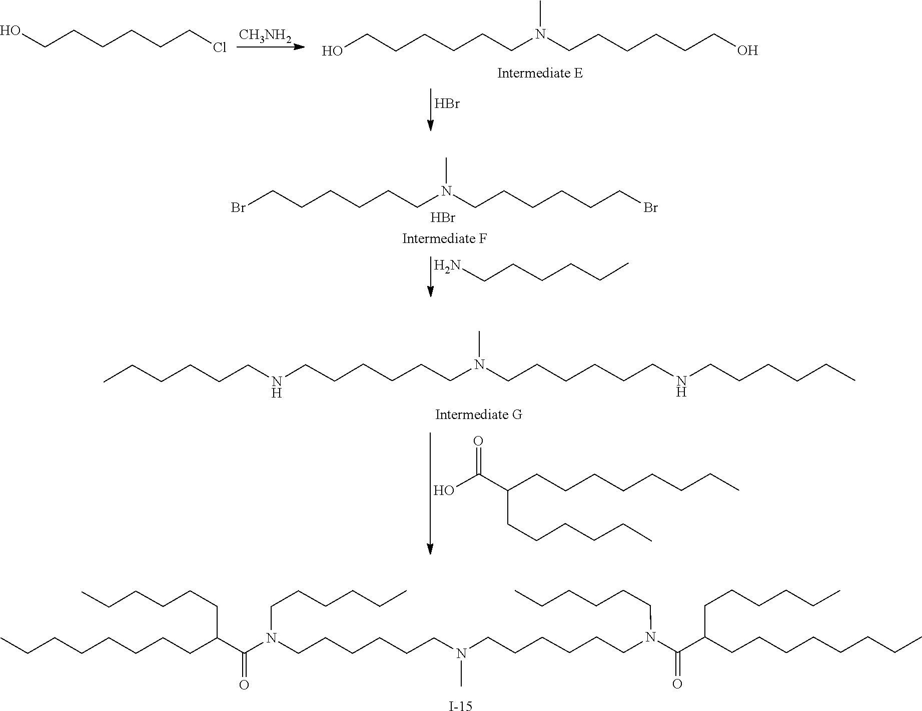

### Synthesis of 6,6′-(methylazanediyl)bis(hexan-1-ol) (Intermediate E)

To a mixture of 6-chloro-1-hexanol (22.8 mmol, 3.11 g, 3.04 mL), absolute ethanol (60 mL), potassium carbonate (2 eq, 24 mmol, 3.32 g), cesium carbonate (eq, 500 mg) and sodium iodide (40 mg) was added methylamine (2 mmol, 1.49 ml, 33 wt. % in absolute ethanol). The mixture was heated for 16 h in a sealed pressure flask (oil bath: 68° C.). The reaction was monitored by TLC. More methylamine (0.1 mL), NaI (300 mg) and potassium carbonate (1 g) were added to the reaction mixture. Heating was resumed and after an additional 4 days, then more 6-chloro-1-hexanol (1.5 mL) and NaI (220 mg) were added. Heating was continued at 76° C. for an additional 3 days. Finally, the mixture was cooled and filtered. The filtrate was concentrated, and the residue was taken up DCM and filtered. The filtrate was concentrated to dryness under reduced pressure. This gave 4.86 g brown viscous oil. The oil was taken up in DCM (100 mL) and loaded on a short column of silica gel under reduced pressure. The column was eluted with a mixture of DCM, methanol and conc. ammonia solution (100:0:0 to 85:15:1). Fractions containing the desired product were combined and concentrated. The residue was taken up in DCM and filtered. The filtrated was concentrated to dryness to give the desired product as brown viscous oil (1.40 g, 6.05 mmol, 50%). The product was used for the next step without further purification.

### Synthesis N¹-hexyl-N⁶-(6-(hexylamino)hexyl)-N⁶-methylhexane-1,6-diamine (Intermediate G)

Hydrobromic acid (6 mL, 48 wt. % solution in water) was added slowly to Intermediate E (1.40 g, 6.05 mmol) with stirring over 5 minutes. The reaction mixture was then heated at 105° C. (oil bath) for 2 hrs. The reaction mixture was cooled a little bit and then toluene (50 mL) was slowly added. The reaction was heated to reflux, and water was removed azeotropically. The reaction mixture was cooled to RT and then transferred to a pressure flask and concentrated to dryness under reduced pressure (brown oil 2.62 g, 5.97 mmol, 98%). TLC (CHCl₃/MeOH=9:1) showed a major spot of 6-bromo-N-(6-bromohexyl)-N-methylhexan-1-amine (Intermediate F). The product was used for the next step without further purification.

A mixture of 6-bromo-N-(6-bromohexyl)-N-methylhexan-1-amine (Intermediate F, 2.62 g, 5.97 mmol), hexylamine (10 eq. 120 mmol, 12.14 g), N,N-diisopropylethylamine (6.0 mmol, 1.04 mL), and sodium iodide (20 mg) in acetonitrile (30 mL) was sealed and heated at 76° C. (oil bath) for 24 h. The mixture was then concentrated at about 75° C. (ca 9 mmHg) to remove solvent and the excess hexylamine. the residue (yellow oil/solid) was taken up in DCM and filtered. TLC showed that hexylamine was not removed completely. The filtrate was concentrated under reduced pressure, giving a yellow foam 4.33 g. The residue was taken up in NaOH solution (1.44 g of sodium hydroxide in 10 mL of water) and then concentrated at 75° C. for 1.5 hrs under reduced pressure. The residue was taken up DCM and filtered. The filtrate was concentrated under reduced pressure to dryness, giving a pale paste (2.671 g, >100%). The product was used for the next step without further purification.

### Synthesis of I-15

To a solution of 2-hexyldecanoic acid (4 mmol, 1.03 g) in DCM (15 mL) and DMF (3 drops from a middle size needle) was added dropwise oxalyl chloride (1.5 eq, 6 mmol, 762 mg, 0.524 mL) at RT. The resulting mixture was then stirred at RT overnight. Next, the mixture was concentrated under reduced pressure at RT. The residue (light yellow paste) was dissolved in 8 mL of DCM and added to a solution of crude Intermediate G (0.67 g) and triethylamine (3 eq, 3.1 mmol, 308 mg, 0.42 mL) and DMAP (5 mg) in DCM (5 mL) at RT in ca 5 min. After addition, the resulting mixture was stirred at RT for 2 h and then concentrated. The residue was taken up in a mixture of hexane, ethyl acetate and Et3N (80:20:1) and was filtered through a pad of silica gel. The pad was washed with the same solvent mixture. Concentration of the filtrate and washings gave a brownish oil (ca 543 mg). The crude product was purified by flash dry column chromatography on silica gel (0 to 6% MeOH in chloroform with a trace of Et₃N). The desired product was obtained as a colorless oil (426 mg, 0.49 mmol, 33%). ¹HNMR (400 MHz, CDCl₃) δ: 3.34-3.27 (m, 4H), 3.26-3.18 (m, 4H), 2.53-2.45 (m, 2H), 2.34-2.25 (m, 4H), 2.18 (t, 4.7 Hz, 3H), 1.65-1.36 (m, 20H), 1.36-1.10 (m, 60H), 0.92-0.84 (m, 18H). ESI-MS: MW for C₅₇H₁₅N₃O₂ [M+H]⁺ Calc. 874.9; Found 875.3.

### Example 25

### N,N′-((Methylazanediyl)Bis(Hexane-6,1-Diyl))Bis(N-Hexylpalmitamide) (Compound I-9)

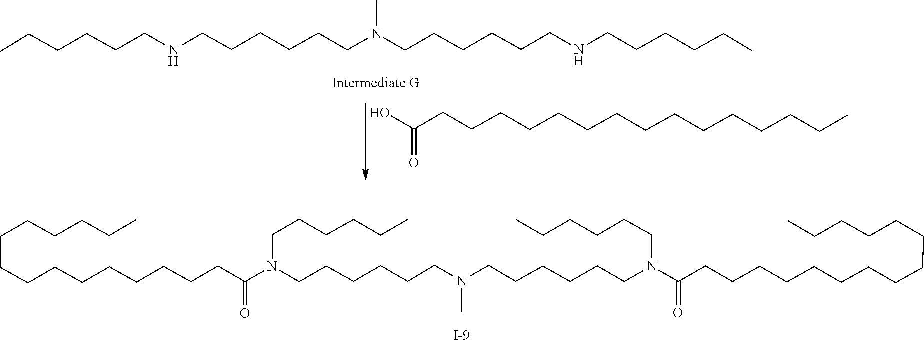

### Synthesis of I-9

To a solution of palmitic acid (2.5 mmol, 0.64 g) in DCM (15 mL) and DMF (2 drops from a small size needle) was added dropwise oxalyl chloride (1.5 eq, 3.75 mmol, 484 mg, 0.33 mL) at RT. The resulting mixture was then stirred at RT overnight. Next, the mixture was concentrated under reduced pressure at RT. The residue (light yellow paste) was dissolved in 8 mL of DCM and added to a solution of crude Intermediate G (300 mg) and triethylamine (0.4 mL) and DMAP (5 mg) in DCM (5 mL) at RT in ca 5 min. After addition, the resulting mixture was stirred at RT for 2 h and then concentrated. The residue was taken up in a mixture of hexane, ethyl acetate and Et3N (80:20:1) and was filtered through a pad of silica gel and the pad was washed with the same solvent mixture. Concentration of the filtrate and washings gave a yellow oil/solid which was taken up in a mixture of hexane, ethyl acetate and Et₃N (80:20:1) and was filtered again through a pad of silica gel and the pad was washed with the same solvent mixture. Concentration of the filtrate and washings gave a yellow oil (ca 244 mg). The crude product was purified by flash dry column chromatography on silica gel (0 to 5% MeOH in chloroform with a trace of Et₃N). The desired product was obtained as a colorless oil (200 mg, 0.22 mmol, 34%). ¹HNMR (400 MHz, CDCl3 at 7.26 ppm) δ: 3.32-3.25 (m, 4H), 3.23-3.15 (m, 4H), 2.33-2.24 (m, 8H), 2.18 (t, 4.2 Hz, 3H), 1.67-1.56 (m, 4H), 1.56-1.36 (m, 12H), 1.36-1.10 (m, 68H), 0.92-0.85 (m, 12H). ESI-MS: MW for C₅₇H₁₁₅N₃O₂ [M+H]⁺ Calc. 874.9; Found 875.1.

### Example 26

### (9Z,9′Z,12Z,12′Z)—N,N′-((Methylazanediyl)Bis(Hexane-6,1-Diyl))Bis(N-Hexyloctadeca-9,12-Dienamide) (Compound I-14)

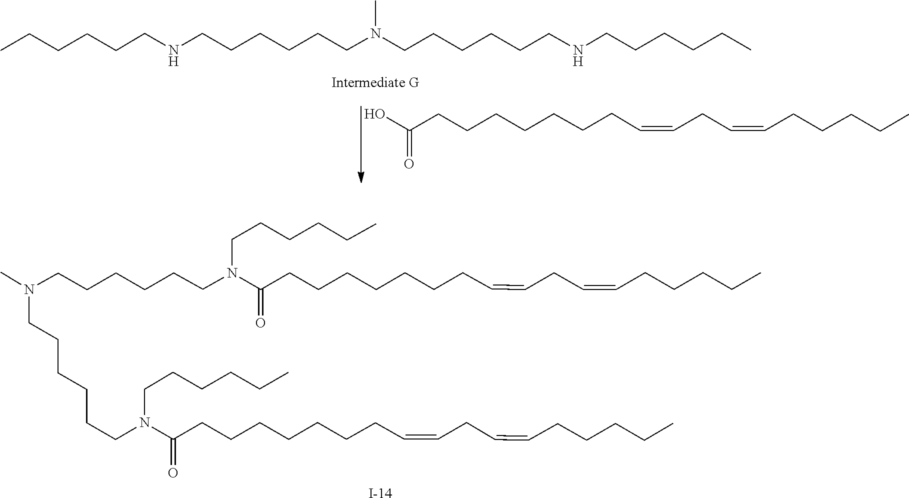

### Synthesis of I-14

To a solution of linoleic acid (2.5 mmol, 0.70 g) in DCM (12 mL) and DMF (2 drops from a small size needle) was added dropwise oxalyl chloride (1.5 eq, 3.75 mmol, 484 mg, 0.33 mL) at RT. The resulting mixture was then stirred at RT overnight. Next, the mixture was concentrated under reduced pressure at RT. The residue (light yellow paste) was dissolved in 8 mL of DCM and added to a solution of crude Intermediate G (300 mg) and triethylamine (0.4 mL) and DMAP (5 mg) in DCM (5 mL) at RT in ca 5 min. After addition, the resulting mixture was stirred at RT for 2 h and then concentrated. The residue was taken up in a mixture of hexane, ethyl acetate and Et₃N (80:20:1) and was filtered through a pad of silica gel and the pad was washed with the same solvent mixture. Concentration of the filtrate and washings gave a yellow oil/solid) which was taken up in hexane and filtered again to remove the solid. The crude product was purified by flash dry column chromatography on silica gel (0 to 5% MeOH in chloroform with a trace of Et₃N). The desired product was obtained as a colorless oil (160 mg, 0.17 mmol, 25%). ¹HNMR (400 MHz, CDCl₃) δ: 5.42-5.29 (m, 8H), 3.32-3.24 (m, 4H), 3.22-3.15 (m, 4H), 2.77 (t, 6.4 Hz, 4H), 2.33-2.24 (m, 8H), 2.19 (t, 4.0 Hz, 3H), 2.05 (q, 6.8 Hz, 8H), 1.67-1.56 (m, 4H), 1.56-1.40 (m, 12H), 1.40-1.10 (m, 46H), 0.92-0.85 (m, 12H). ESI-MS: MW for C₆₁H₁₁₅N₃O₂ [M+H]⁺ Calc. 922.9; Found 923.1.

### Example 27

### N,N-Didecyl-8-((8-(Hexadecylamino)-8-Oxooctyl)(Methyl)Amino)Octanamide (Compound I-12)

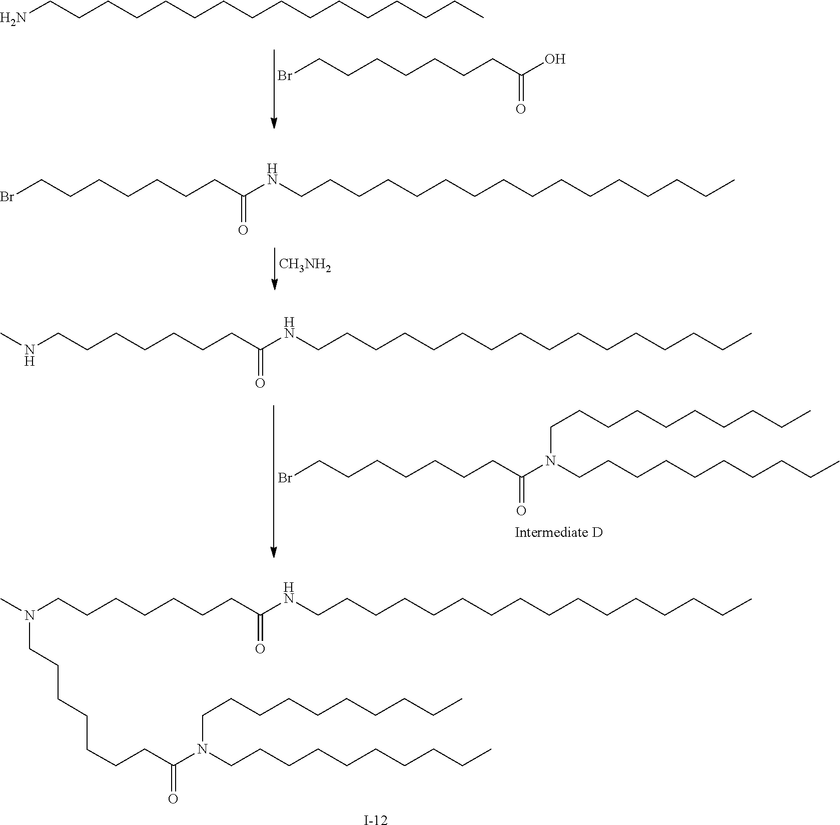

### Synthesis of 8-bromo-N-hexadecyloctanamide

To a mixture of 8-bromohexanoic acid (1.0 equiv, 2.53 g, 11.36 mmol), and 4-dimethylaminopyridine (0.3 eq., 3.41 mmol, 416 mg) and N-hydroxysuccinimide (1.0 equiv, 11.36 mmol, 1.31 g) in 20 mL of DCM was added DCC (1.05 equiv, 11.93 mmol, 2.46 g) and the mixture stirred at room temperature for 2.5 h. The reaction mixture was filtered into a flask containing hexadecylamine (1 equiv, 11.36 mmol, 2.743 g) and washed with DCM (ca 20 mL). The resulting mixture was stirred at RT overnight. After concentration of the reaction mixture, a white solid was obtained. The solid was taken up in DCM (100 mL) and ultrasonicated to get a slightly cloudy solution. The cloudy solution was loaded on a short silica gel column under reduced pressure. The column was eluted with a mixture of DCM and MeOH (100:0 to 98.75:1.25) under reduced pressure. The desired product was obtained as a white solid (4.50 g, 10.1 mmol, 89%).

### Synthesis of N-hexadecyl-8-(methylamino)octanamide

A mixture of methylamine (1 mL, 8 mmol, 33 wt. % in absolute ethanol), 8-bromo-N-hexadecyloctanamide (1 eq. 288 mg, 0.61 mml) and K₂CO₃ (1 mmol, 138 mg,) in 10 mL of EtOH was sealed in a pressure bottle and heated at 85° C. (oil bath) overnight. After concentration of the mixture, the residue was taken up in a mixture of DCM, MeOH and Et3N (90:10:0.5) and filtered. Concentration of the filtrate and washings gave a white solid (228 mg, 0.57 mmol, 89%) which was used for the next step without further purification.

### Synthesis of I-12

A mixture of crude N-hexadecyl-8-(methylamino)octanamide (228 mg), anhydrous acetonitrile (15 mL), N,N-diisopropylethylamine (0.33 mL) and Intermediate D (327 mg, 0.65 mmol) was heated at 74° C. (oil bath) for 16 h in a pressure flask. Then the reaction mixture was concentrated. The residue was taken up in a mixture of hexane, ethyl acetate and Et₃N (80:20:1) and was filtered through a pad of silica gel. The pad was washed with the same solvent mixture. Concentration of the filtrate and washings gave a colorless oil. The crude product was further purified flash dry column chromatography on silica gel using 0 to 5% MeOH in chloroform with a trace of Et₃N as elution solvent mixture. The desired product was obtained as a colorless oil (192 mg, 0.23 mmol, 40%). ¹HNMR (400 MHz, CDCl₃, at 7.26 ppm) δ: 5.45 (s, 1H), 3.30-3.16 (m, 6H), 2.30-2.24 (m, 6H), 2.18 (s, 3H), 2.14 (t, 7.6 Hz, 2H), 1.67-1.57 (m, 4H), 1.54-1.37 (m, 10H), 1.37-1.16 (m, 66H), 0.91-0.86 (m, 9H). ESI-MS: MW for C₅₃H₁₀₇N₃O₂ [M+H]⁺ Calc. 818.8; Found 819.0.

### Example 28

### 8,8′-((8-(Decylamino)-8-Oxooctyl)Azanediyl)Bis(N,N-Didecyloctanamide) (Compound I-33)

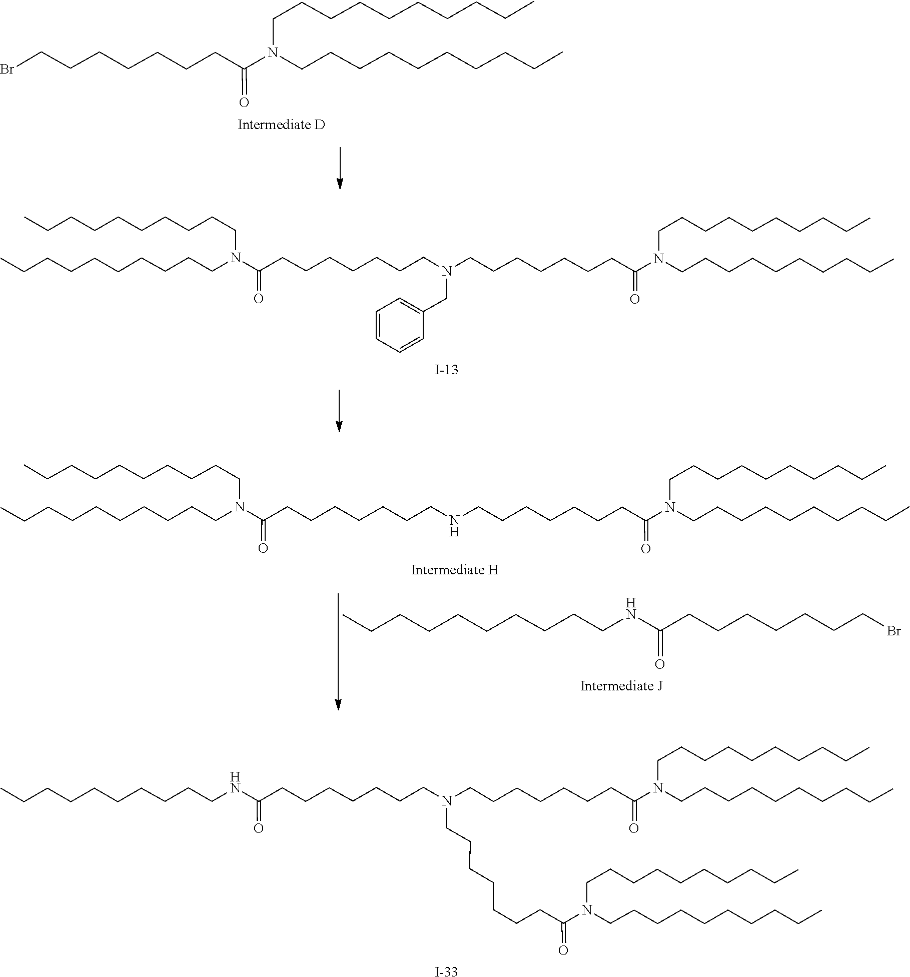

### Synthesis of I-13

To a mixture of Intermediate D (1.9 eq, 2.0 g, 3.98 mmol), anhydrous acetonitrile (25 mL) and N,N-diisopropylethylamine (2.08 mL) in a pressure flask was added benzylamine (2.09 mmol, 224 mg, 0.23 mL). the resulting mixture was heated at 75° C. (oil bath) overnight. Then the reaction mixture was concentrated. The crude product was purified by flash dry column chromatography on silica gel (hexane/EtOAc/Et₃N, 90:10:0 to 80:20:1). Concentration of the fractions containing the desired product gave a brown oil (1.43 g) which was pure enough to use for the next step without further purification. A small amount of the product was further purified by flash dry column chromatography on silica gel using 0 to 5% MeOH in chloroform with a trace of Et₃N as elution solvent mixture for analysis and testing. ¹HNMR (400 MHz, CDCl₃, at 7.26 pp) δ: 7.33-7.18 (m, 5H), 3.52 (s, 2H), 3.31-3.24 (m, 4H), 3.22-3.14 (m, 4H), 2.41-2.33 (m, 4H), 2.29-2.21 (m, s4H), 1.61 (quintet, 7.3 Hz, 4H), 1.56-1.38 (m, 12H), 1.38-1.10 (m, 68H), 0.90-0.85 (m, 12H).

### Synthesis of 8,8′-azanediylbis(N,N-didecyloctanamide) (Intermediate H)

A mixture of I-13 (1.18 g, 1.24 mmol), 10% Pd/C (39 mg) in methanol (15 mL) was stirred under hydrogen for 3 days at RT. The reaction mixture was filtered through a pad of Celite© and washed with MeOH. The filtrate was concentrated (1.092 g colorless oil/solid) which was used for the next step without further purification.

### Synthesis of 8-bromo-N-decyloctanamide (Intermediate J)

To a mixture of 8-bromohexanoic acid (1.0 equiv, 2.53 g 11.36 mmol), and 4-dimethylaminopyridine (0.3 eq., 3.41 mmol, 416 mg) in 20 mL of DCM was added N-hydroxysuccinimide (1.0 equiv, 11.36 mmol, 1.31 g), followed by addition of DCC (1.05 equiv, 11.93 mmol, 2.46 g). After the resulting mixture was stirred at room temperature for 2.5 h, the mixture was filtered into a flask containing decylamine (1 equiv, 11.36 mmol, 1.79 g, 2.27 mL) and the solid was washed with more DCM (3 mL×2). The resulting mixture of the filtrate and decylamine was stirred at RT overnight. The next day the mixture was concentrated. The residue was taken up in DCM and loaded onto a silica gel column. The column was eluted with a mixture of DCM and MeOH (100:0 to 97.5:2.5). The desired product was obtained as white solid (3.447 g, 9.51 mmol, 84%).

### Synthesis of I-33

A mixture of 8,8′-azanediylbis(N,N-didecyloctanamide) (Intermediate H, 300 mg, 0.35 mmol), 8-bromo-N-decyloctanamide (Intermediate J, 1 eq, 0.35 mmol, 127 mg), anhydrous acetonitrile (15 mL), N,N-diisopropylethylamine (0.12 mL), and NaI (44 mg) in a pressure flask was heated at 73° C. (oil bath) overnight. Then the reaction mixture was concentrated. The residue was taken up in a mixture of hexane, ethyl acetate and Et₃N (80:20:1) and was filtered through a pad of silica gel. The pad was washed with the same solvent mixture. Concentration of the filtrate and washings gave a yellow oil. The crude product was further purified by flash dry column chromatography on silica gel using 0 to 5% MeOH in chloroform with a trace of Et₃N as elution solvent mixture. The desired product was obtained as a colorless oil (212 mg, 0.19 mmol, 53%). ¹HNMR (400 MHz, CDCl₃, at 7.26 pp) δ: 5.62 (br. t, 5 Hz, 1H), 3.31-3.24 (m, 4H), 3.24-3.16 (m, 6H), 2.40-2.31 (m, 6H), 2.30-2.23 (m, 4H), 2.15 (t, 7.6 Hz, 2H), 1.67-1.57 (m, 6H), 1.55-1.36 (m, 16H), 1.36-1.10 (m, 88H), 0.90-0.85 (m, 15H). ESI-MS: MW for C₇₄H₁₄₈N₄O₃ [M+H]⁺ Calc. 1142.2; Found 1142.2.

### Example 29

### 8,8′-((6-(Dihexylamino)-6-Oxohexyl)Azanediyl)Bis(N,N-Didecyloctanamide) (Compound I-32)

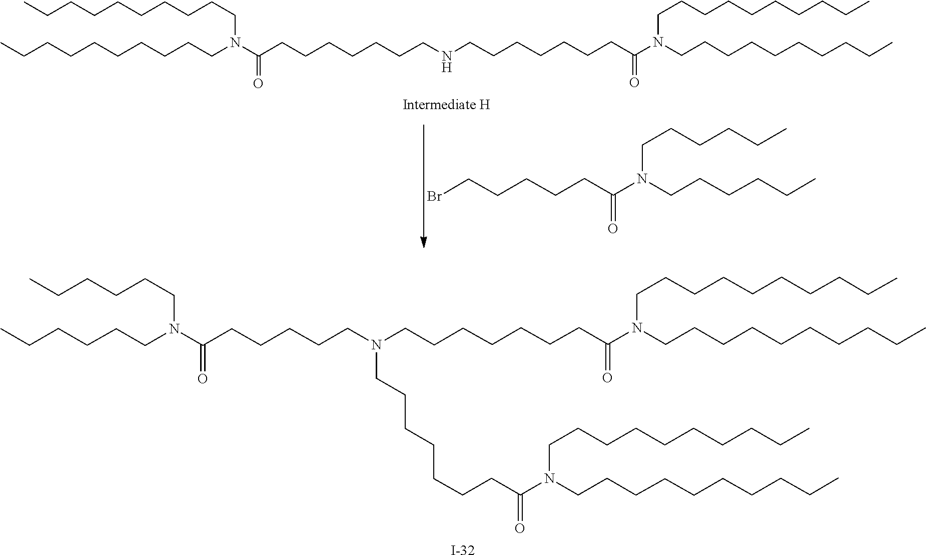

### Synthesis of I-32

A mixture of 6-bromo-N,N-dihexylhexanamide (1 eq, 0.35 mmol, 127 mg; made from 6-bromohexanoic acid and dihexylamine in a similar way to Intermediate D), anhydrous acetonitrile (15 mL), N,N-diisopropylethylamine (0.12 mL), 8,8′-azanediylbis(N,N-didecyloctanamide) (Intermediate H, 300 mg, 0.35 mmol) and NaI (44 mg) in a pressure flask was heated at 80° C. (oil bath) overnight. Then the reaction mixture was concentrated. The residue was taken up in a mixture of hexane, ethyl acetate and Et₃N (80:20:1) and was filtered through a pad of silica gel. The pad was washed with the same solvent mixture. Concentration of the filtrate and washings gave a yellow oil. The crude product was further purified by flash dry column chromatography on silica gel using 0 to 5% MeOH in chloroform with a trace of Et₃N as elution solvent mixture. The desired product was obtained as a slightly yellow oil (228 mg, 0.20 mmol, 57%). ¹HNMR (400 MHz, CDCl₃ at 7.26 ppm) δ: 3.31-3.24 (m, 6H), 3.22-3.15 (m, 6H), 2.40-2.33 (m, 6H), 2.30-2.24 (m, 6H), 1.69-1.58 (m, 6H), 1.54-1.36 (m, 18H), 1.36-1.10 (m, 82H), 0.92-0.85 (m, 18H). ESI-MS: MW for C₇₄H₁₄₈N₄O₃ [M+H]⁺ Calc. 1142.2; Found 1142.2.

### Example 30

### 8,8′-((5-(Decylamino)-5-Oxopentyl)Azanediyl)Bis(N,N-Didecyloctanamide) (Compound I-30)

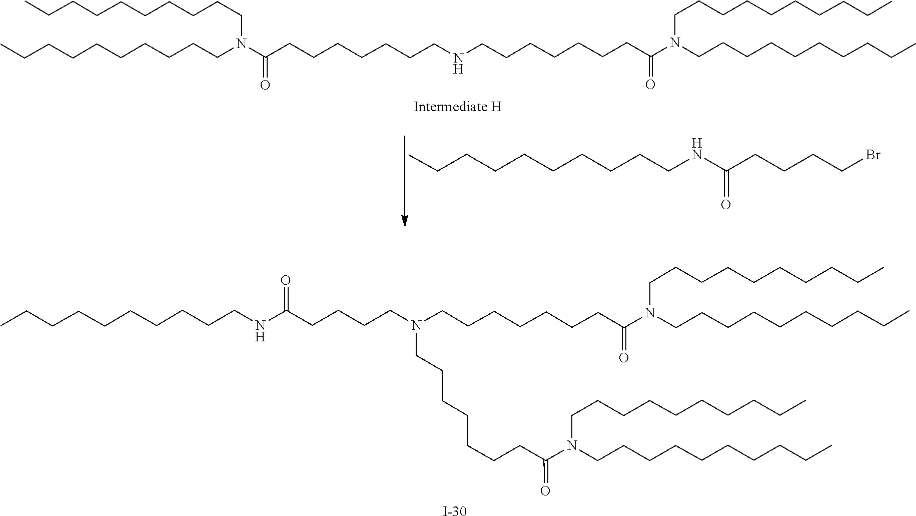

### Synthesis of 5-bromo-N-decylpentanamide

To a mixture of 5-bromovaleric acid (1.0 equiv, 2.06 g 11.36 mmol), and 4-dimethylaminopyridine (0.3 eq., 3.41 mmol, 416 mg) in 20 mL of DCM was added N-hydroxysuccinimide (1.0 equiv, 11.36 mmol, 1.31 g), followed by addition of DCC (1.05 equiv, 11.93 mmol, 2.46 g). After the resulting mixture was stirred at room temperature for 2.5 h, the mixture was filtered into a flask containing decylamine (1 equiv, 11.36 mmol, 1.79 g, 2.27 mL) and the solid was washed with more DCM (3 mL×2). The resulting mixture of the filtrate and decylamine was stirred at RT overnight. The next day the mixture was concentrated. The residue was taken up in DCM and loaded onto a silica gel column. The column was eluted with a mixture of DCM and MeOH (100:0 to 98:2). The desired product was obtained as white solid (2.758 g, 8.62 mmol, 76%).

### Synthesis of I-30

A mixture of 8,8′-azanediylbis(N,N-didecyloctanamide) (Intermediate H, 246 mg, 0.27 mmol), 5-bromo-N-decylpentanamide (130 mg), anhydrous acetonitrile (12 mL), N,N-diisopropylethylamine (0.10 mL) and NaI (40 mg) in a pressure flask was heated at 73° C. (oil bath) overnight. After the reaction mixture was concentrated, the residue was taken up in a mixture of hexane, ethyl acetate, MeOH and Et₃N (80:20:2:1) and was filtered through a pad of silica gel. The pad was washed with the same solvent mixture. Concentration of the filtrate and washings gave a yellow oil. The crude product was further purified by flash dry column chromatography on silica gel using 0 to 5% MeOH in chloroform with a trace of Et₃N as elution solvent mixture. The desired product was obtained as a colorless oil (60 mg). ¹HNMR (400 MHz, CDCl₃ at 7.26 ppm) δ: 5.75 (br. t, 5 Hz, 1H), 3.33-3.13 (m, 10H), 2.41-2.32 (m, 6H), 2.27 (t, 7.6 Hz, 4H), 2.17 (t, 7.6 Hz, 2H), 1.67-1.57 (m, 6H), 1.55-1.35 (m, 16H), 1.35-1.16 (m, 82H), 0.90-0.85 (m, 15H).

### Example 31

### 6,6′-((8-(Decylamino)-8-Oxooctyl)Azanediyl)Bis(N,N-Didecylhexanamide) (Compound I-29)

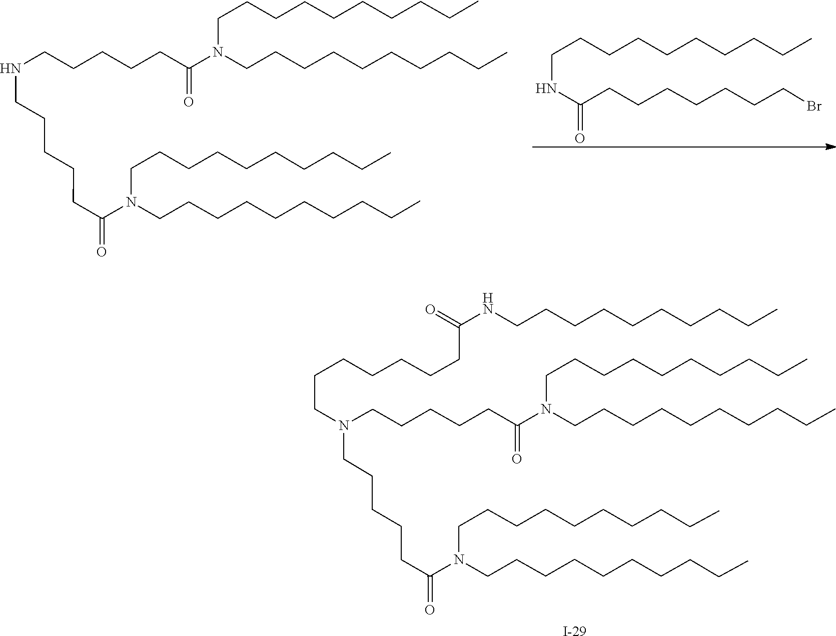

### Synthesis of I-29

A mixture of 6,6′-azanediylbis(N,N-didecylhexanamide) (300 mg, 0.37 mmol; made in a similar way to Intermediate H), 8-bromo-N-decyloctanamide (1 eq, 0.37 mmol, 135 mg), anhydrous acetonitrile (15 mL), N,N-diisopropylethylamine (0.12 mL), and NaI (70 mg) in a pressure flask was heated at 73° C. (oil bath) overnight. After the reaction mixture was concentrated, the residue was taken up in a mixture of hexane, ethyl acetate, MeOH and Et₃N (80:20:2:1) and was filtered through a pad of silica gel. The pad was washed with the same solvent mixture. Concentration of the filtrate and washings gave a yellow oil. The crude product was further purified flash dry column chromatography on silica gel using 0 to 5% MeOH in chloroform with a trace of Et₃N as elution solvent mixture. The desired product was obtained as a colorless oil (254 mg, brownish oil, 0.23 mmol, 63%). ¹HNMR (400 MHz, CDCl₃ at 7.26 ppm) δ: 5.62 (br. t, 5 Hz, 1H), 3.34-3.16 (m, 10H), 2.40-2.33 (m, 6H), 2.27 (t, 7.6 Hz, 4H), 2.15 (t, 7.6 Hz, 2H), 1.68-1.57 (m, 6H), 1.56-1.36 (m, 16H), 1.36-1.10 (m, 80H), 0.90-0.85 (m, 15H). ESI-MS: MW for C₇₀H₁₄₀N₄O₃ [M+H]⁺ Calc. 1086.1; Found 1086.3.

### Example 32

### 6,6′-((2-(Dihexylamino)Ethyl)Azanediyl)Bis(N,N-Didecylhexanamide) (Compound I-31)

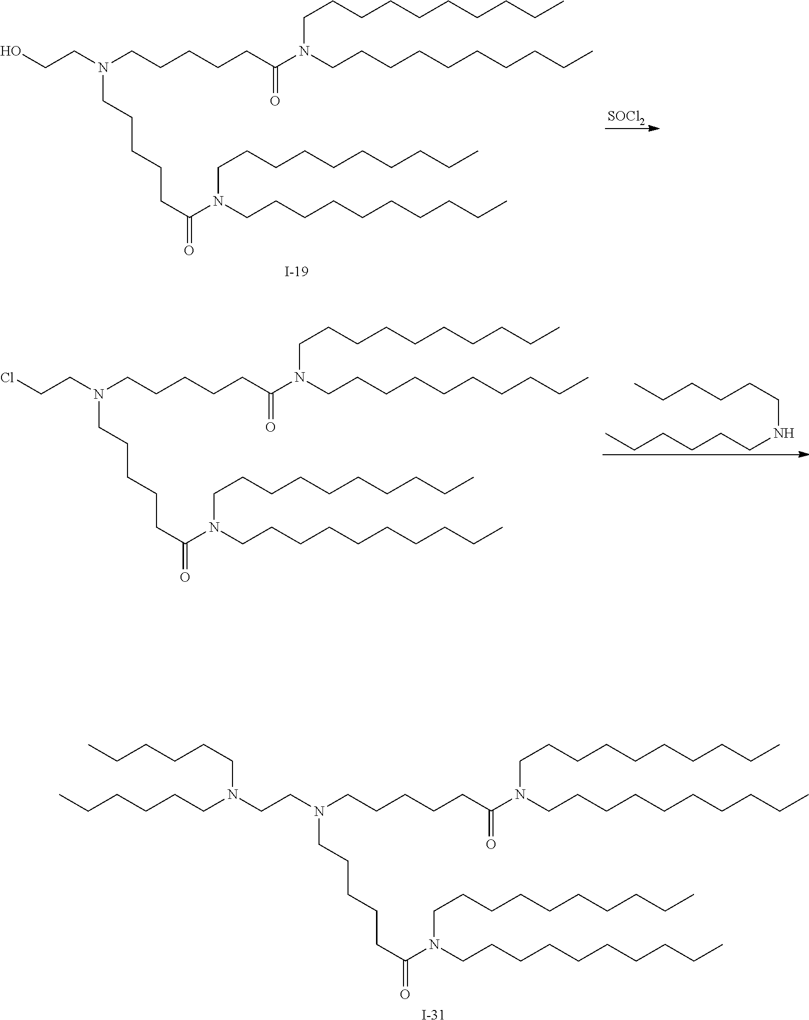

### Synthesis of 6,6′-((2-chloroethyl)azanediyl)bis(N,N-didecylhexanamide)

To an ice-cooled solution of I-19 (333 mg, 0.39 mmol) of in 2 mL of CHCl₃, was added dropwise a solution of thionyl chloride (200 mg) in 10 mL of chloroform under Ar. After the completion of addition of SOCl₂ (10 min), the ice bath was removed and the reaction mixture was stirred for 16 h at room temperature. The next day removal of CHCl₃ and SOCl₂ under reduced pressure gave brown oil. The residual oil was taken up in a mixture of hexane, ethyl acetate and Et₃N, (ca (80:20:1) and was filtered through a pad of silica gel. The pad was washed with the same solvent mixture. Concentration of the filtrate and washings gave a yellow oil (282 mg, 0.325 mmol, 83%). The product was used for the next step without further purification.

### Synthesis of I-31

A mixture of 6,6′-((2-chloroethyl)azanediyl)bis(N,N-didecylhexanamide) (282 mg, 0.33 mmol), dihexylamine (5 eq. 1.6 mmol, 1.066 g), N,N-diisopropylethylamine (2 equiv., 0.66 mmol, 85 mg, 0.11 mL; MW129.25, d 0.742), and sodium iodide (60 mg) in acetonitrile (10 mL) was sealed and heated at 76° C. for 24 h. After the reaction mixture was concentrated, the residue was taken up in a mixture of hexane, ethyl acetate, MeOH and Et₃N (80:20:1:1) and was filtered through a pad of silica gel. The pad was washed with the same solvent mixture. Concentration of the filtrate and washings gave a yellow oil. The yellow oil was treated with a solution of acetyl chloride and Et₃N in DCM at RT to transfer the unreacted dihexylamine to an amide. After removal of DCM, the crude product was purified by flash dry column chromatography on silica gel (hexane/ethyl acetate/Et₃N, 9:1 to 80:20:1; 0 to 5% MeOH in chloroform with a trace of Et3N). The desired product was obtained as a colorless oil (256 mg, colorless oil, 0.25 mmol, 76%). ¹HNMR (400 MHz, CDCL₃ at 7.26 ppm) δ: 3.31-3.24 (m, 4H), 3.23-3.15 (m, 4H), 2.50 (s, 4H), 2.45-2.36 (m, 8H), 2.27 (t, 7.6 Hz, 4H), 1.64 (quintet, 7.6 Hz, 4H), 1.59-1.36 (m, 16H), 1.36-1.10 (m, 72H), 0.92-0.84 (m, 18H). ESI-MS: MW for C₆₆H₁₃₄N₄O₂ [M+H]⁺ Calc. 1016.1; Found 1016.2.

### Example 33

### 10,10′-((2-(Dimethylamino)Ethyl)Azanediyl)Bis(N,N-Didecyldecanamide) (Compound I-28)

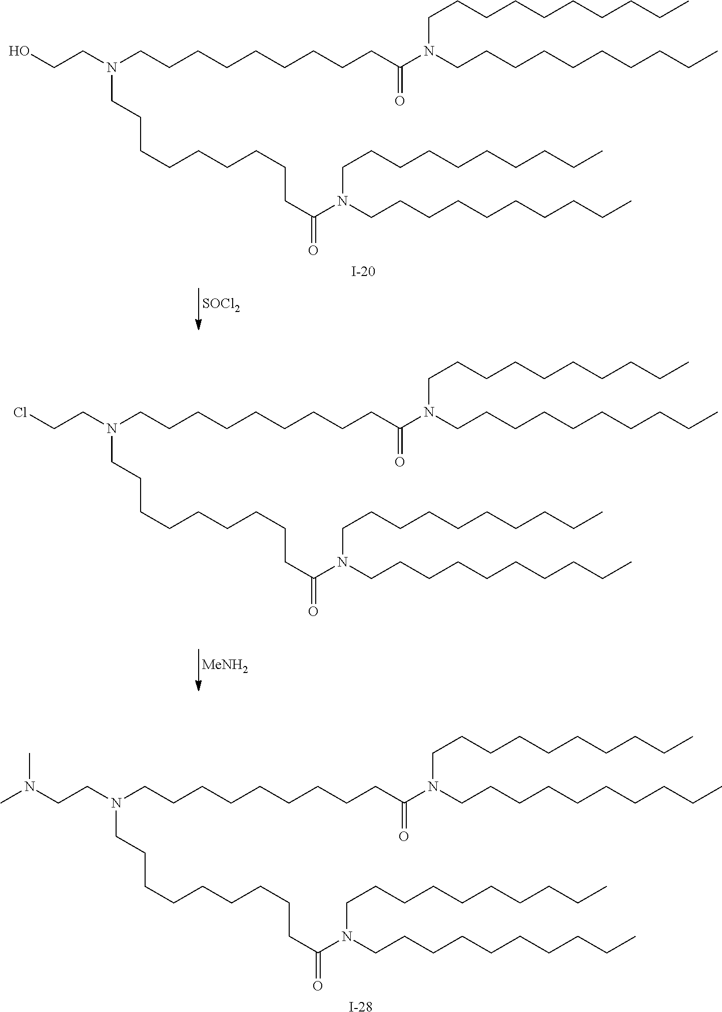

### Synthesis of 10,10′-((2-chloroethyl)azanediyl)bis(N,N-didecyldecanamide)

To an ice-cooled solution of I-20 (293 mg, 0.30 mmol) of in 2 mL of CHCl₃, was added dropwise a solution of thionyl chloride (1.17 mmol, 139 mg, 0.085 mL) in 10 mL of chloroform. After the completion of addition of SOC₂ (10 min), the ice bath was removed and the reaction mixture was stirred for 16 h at room temperature. The next day removal of CHCl3, and SOCl2 under reduced pressure gave brown oil. The residual oil was taken up in a mixture of hexane, ethyl acetate and Et₃N, (ca (80:20:1) and was filtered through a pad of silica gel. The pad was washed with the same solvent mixture. Concentration of the filtrate and washings gave a yellow oil (278 mg, 0.28 mmol, 95%). The product was used for the next step without further purification.

### Synthesis of I-28

A mixture of 10,10′-((2-chloroethyl)azanediyl)bis(N,N-didecyldecanamide) (278 mg, 0.28 mmol), dimethylamine (2M in THF, 10 mL) and N,N-diisopropylethylamine (0.66 mmol) in a pressure flask was sealed and heated at 75° C. for 24 h. After the reaction mixture was concentrated, the residue was taken up in a mixture of hexane, ethyl acetate, MeOH and Et₃N (80:20:1:1) and was filtered through a pad of silica gel. The pad was washed with the same solvent mixture. Concentration of the filtrate and washings gave a yellow oil. The crude product was purified by flash dry column chromatography on silica gel (0 to 5% MeOH in chloroform with a trace of Et₃N). The desired product was obtained as a colorless oil (244 mg, slightly yellow oil, 0.25 mmol, 76%). ¹HNMR (400 MHz, CDCl₃ at 7.26 ppm) δ: 3.31-3.25 (m, 4H), 3.22-3.15 m, 4H), 2.56-2.551 (m, 2H), 2.43-2.33 (m, 6H), 2.26 (t, 7.6 Hz, 4H), 2.23 (s, 6H), 1.67-1.59 (m, 4H), 1.56-1.45 (m, 8H), 1.45-1.36 (m, 4H), 1.36-1.15 (m, 76H), 0.90-0.85 (m, 12H). ESI-MS: MW for C₆₄H₁₃₀N₄O₂[M+H]⁺ Calc. 988.0; Found 988.1.

### Example 34

### 2-Butyloctyl 6-(Bis(6-(Dioctylamino)-6-Oxohexyl)Amino)Hexanoate (Compound I-34)

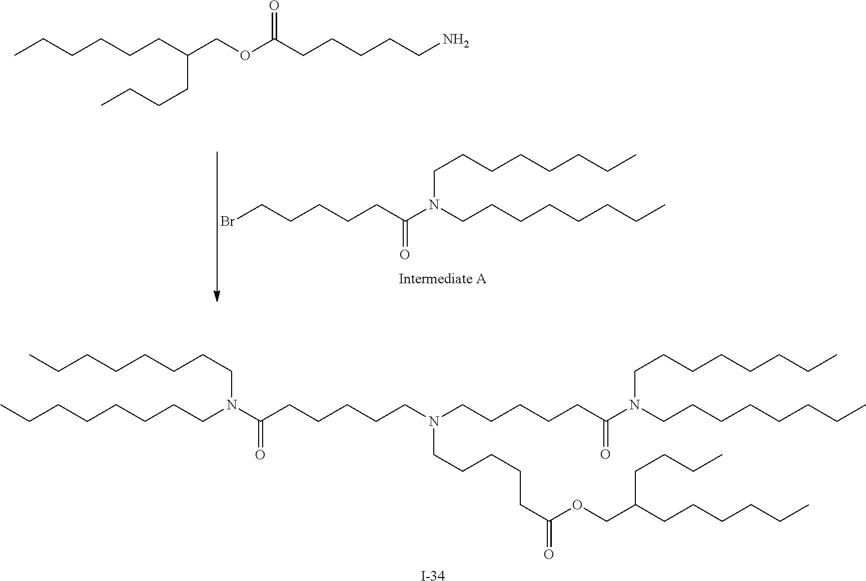

### Synthesis of I-34

A mixture of 2-butyloctyl-6-aminohexanoate (0.76 mmol, 228 mg; made from 2-butyl-1-octanol and 6-aminocaproic acid; the purity is less than 80%), 6-bromo-N,N-dioctylhexanamide (Intermediate A, 1.44 mmol, 603 mg), anhydrous acetonitrile (15 mL) and N,N-diisopropylethylamine (0.4 mL) in a pressure flask was sealed and heated at 80° C. for 16 h. After the reaction mixture was concentrated, the residue was taken up in a mixture of hexane, ethyl acetate, and Et₃N (80:20:1) and was filtered through a pad of silica gel. The pad was washed with the same solvent mixture. Concentration of the filtrate and washings gave a yellow oil. The crude product was purified by flash dry column chromatography on silica gel (0 to 5% MeOH in chloroform with a trace of Et3N). The desired product was obtained as a colorless oil (172 mg, colorless oil, 0.18 mmol, 25%). ¹HNMR (400 MHz, CDCl₃ at 7.26 ppm) δ: 3.97 (d, 5.8 Hz, 2H), 3.33-3.24 (m, 4H), 3.24-3.15 (m, 4H), 3.02-2.91 (br., 1H), 2.42 (m, 5H), 2.33-2.25 (m, 6H), 1.86-1.74 (br. 1H), 1.70-1.36 (m, 20H), 1.36-1.16 (m, 62H), 0.91-0.85 (m, 18H).

### Example 35

### 6,6′-((4-Hydroxybutyl)Azanediyl)Bis(N,N-Bis(2-Ethylhexyl)Hexanamide) (Compound 1-27)

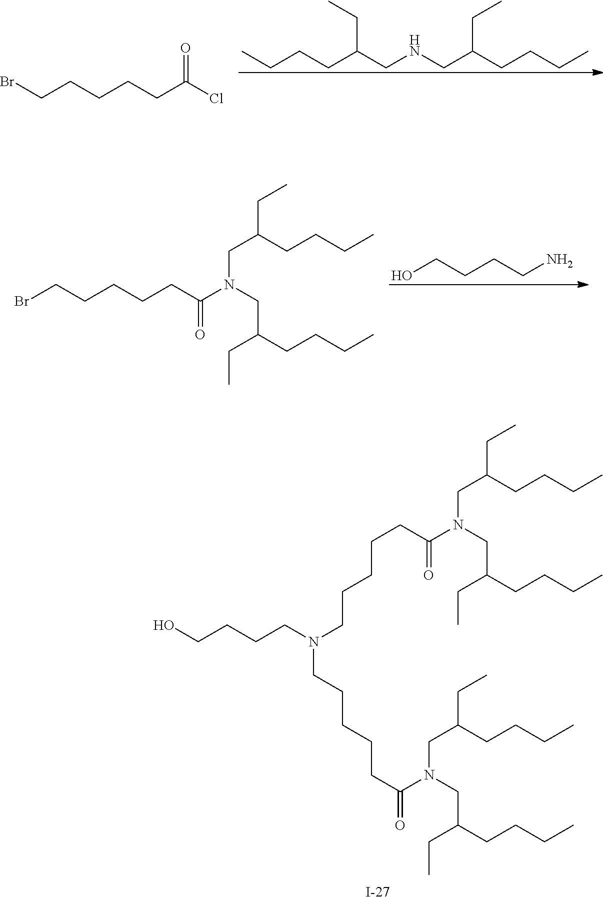

### Synthesis of I-27

A solution of bis-(2-ethylhexyl)amine (7.5 g) and 6-bromohexanoyl chloride (6.1 g) in dichloromethane (60 mL) was treated with triethylamine (6 mL) and stirred for two hours. The solution was washed with dilute hydrochloric acid. The organic fractions were dried over anhydrous magnesium sulphate, filtered and the solvent removed. The crude product was passed down a silica gel column using dichloromethane to remove polar impurities. A solution of the resultant product (3 g) in THF was treated with 4-aminobutan-1-ol (0.22 g) and N,N-diisopropylethylamine (1 mL). The reaction was refluxed for three days, then partitioned between dilute hydrochloric acid and dichloromethane. The solvent was removed from the organic fraction and the residue passed down a silica gel (55 g) column using a methanol/dichloromethane gradient, yielding I-27 (1.6 g). ¹H NMR (400 MHz, CDCl₃ at 7.26 ppm): 3.57-4.49 (m, 2H), 3.33-3.18 (m, 4H) 3.12 (d, 7.4 Hz, 4H), 2.47-2.26 (m, 10H), 1.74-1.12 (m, 52H), 0.93-0.81 (m, 24H). ESI-MS: MW for C₄₈H₉₇N₃O₃ [M+H]⁺ Calc. 764.8; Found 764.8.

### Example 36

### 8,8′-((2-Hydroxyethyl)Azanediyl)Bis(N,N-Didodecyloctanamide) (Compound I-37)

Compound I-37 was prepared according to the general procedures of example 13 to yield the desired product as a colorless oil (148 mg, 0.15 mmol, 32%). ¹HNMR (400 MHz, CDCl₃ at 7.26 ppm) δ: 3.51 (t, 5.0 Hz, 2H), 3.30-3.25 (m, 4H), 3.22-3.16 (m, 4H), 2.56 (t, 5.3 Hz, 2H), 2.42 (t, 7.4 Hz, 4H), 2.27 (t, 7.6 Hz, 4H), 1.68-1.38 (m, 16H), 1.36-1.15 (84H), 0.90-0.86 (m, 12H). ESI-MS: MW for C₆₆H₁₃₃N₃O₃ [M+H]⁺ Calc. 1017.0; Found 1017.1.

### Example 37

### 6,6′-((6-Hydroxyhexyl)Azanediyl)Bis(N,N-Didodecylhexanamide) (Compound I-38)

Compound I-38 was prepared according to the general procedures of example 13 to yield the desired product as a colorless oil (259 mg, 0.25 mmol, 51%). ¹HNMR (400 MHz, CDCl₃ at 7.26 ppm) δ: 3.67-3.61 (m, 2H), 3.28 (t, 7.6 Hz, 4H), 3.19 (t, 7.7 Hz, 4H), 2.41-2.35 (m, 6H), 2.27 (t, 7.5 Hz, 4H), 1.64 (quintet, 7.6 Hz, 4H), 1.60-1.40 (m, 16H), 1.40-1.10 (m, 80H), 0.90-0.85 (m, 12H). ESI-MS: MW for C₆₆H₁₃₃N₃O₃ [M+H]⁺ Calc. 1017.0; Found 1017.4.

### Example 38

### N,N-Didecyl-8-((8-(Hexadecyl(Methyl)Amino)-8-Oxooctyl)(Methyl)Amino)Octanamide (Compound I-39)

Compound I-39 was prepared according to the general procedures of example 27 to yield the desired product as a colorless oil (152 mg, 0.18 mmol, 46%). ¹HNMR (400 MHz, CDCl₃ at 7.26 ppm) δ: 3.36-3.16 (m, 6H), 2.96, 2.90 (2 sets of singlet, 3H), 2.31-2.24 (m, 8H), 2.18 (s, 3H), 1.68-1.58 (m, 4H), 1.56-1.38 (m, 10H), 1.38-1.18 (m, 66H), 0.91-0.86 (m, 9H). ESI-MS: MW for C₅₄H₁₀₉N₃O₃ [M+H]⁺ Calc. 832.8; Found 832.8.

### Example 39

### 8,8′-(Methylazanediyl)Bis(N,N-Didodecyloctanamide) (Compound I-40)

Compound I-40 was prepared according to the general procedures of example 10 to yield the desired product as a colorless oil (235 mg, 0.24 mmol, 53%). ¹HNMR (400 MHz, CDCl₃ at 7.26 ppm) δ: 3.28 (t, 7.6 Hz, 4H), 3.19 (t, 7.7 Hz, 4H), 2.30-2.24 (m, 8H), 2.18 (s, 3H), 1.63 (quintet, 7.6 Hz, 4H), 1.57-1.43 (m, 12H), 1.37-1.10 (m, 84H), 0.90-0.86 (m, 12H). ESI-MS: MW for C₆₅H₁₃₁N₃O₂ [M+H]⁺ Calc. 987.0; Found 987.4.

### Example 40

### 8,8′-((3-Hydroxypropyl)Azanediyl)Bis(N,N-Didecyloctanamide) (Compound I-41)

Compound I-41 was prepared according to the general procedures of example 13 to yield the desired product (100 mg, 22%). ¹HNMR (400 MHz, CDCl₃ at 7.26 ppm) δ: 3.78 (t, J=5.1 Hz, 2H), 3.32-3.23 (m, 4H), 3.23-3.15 (m, 4H), 2.64 (t, J=5.6 Hz, 2H), 2.44-2.36 (m, 4H), 2.31-2.22 (m, 4H), 1.65 (dp, J=22.1, 6.3 Hz, 6H), 1.58-1.40 (m, 12H), 1.38-1.18 (m, 70H), 0.88 (td, J=6.8, 3.0 Hz, 12H). ESI-MS: MW for C₅₉H₁₁₉N₃O₃ [M+H]⁺ Calc. 918.9; Found 919.3.

### Example 41

### 8,8′-((2-(2-Hydroxyethoxy)Ethyl)Azanediyl)Bis(N,N-Didecyloctanamide) (Compound I-42)

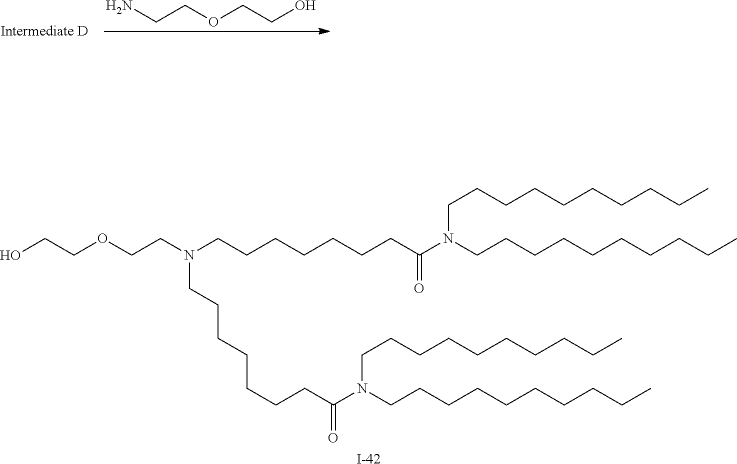

### Synthesis of I-42

A mixture of Intermediate D (1.2 mmol, 600 mg), 2-(2-aminoethoxy)ethan-1-ol (0.74 mmol, 78 mg), and DIEA (3.0 mmol, 0.52 mL) in ACN (7 mL) was heated at 72° C. for 48 h. The reaction mixture was concentrated and the crude material was purified via automated flash chromatography (5% to 60% EtOAc in hexanes with 1% Et₃N) to give compound I-42 (200 mg, 35%). ¹H NMR (400 MHz, CDCl₃) δ 3.68 (m, 3.0 Hz, 2H), 3.63-3.57 (m, 4H), 3.32-3.23 (m, 4H), 3.23-3.14 (m, 4H), 2.62 (t, J=5.4 Hz, 2H), 2.49-2.41 (m, 4H), 2.30-2.22 (m, 4H), 1.61 (m, 4H), 1.48 (m, 12H), 1.38-1.23 (m, 68H), 0.88 (m, 12H). ESI-MS: MW for C₆₀H₁₂₁N₃O₄ [M+H]⁺ Calc. 948.9; Found 949.4.

### Example 42

### 8,8′-((5-Hydroxy-4,4-Dimethylpentyl)Azanediyl)Bis(N,N-Didecyloctanamide) (Compound I-43)

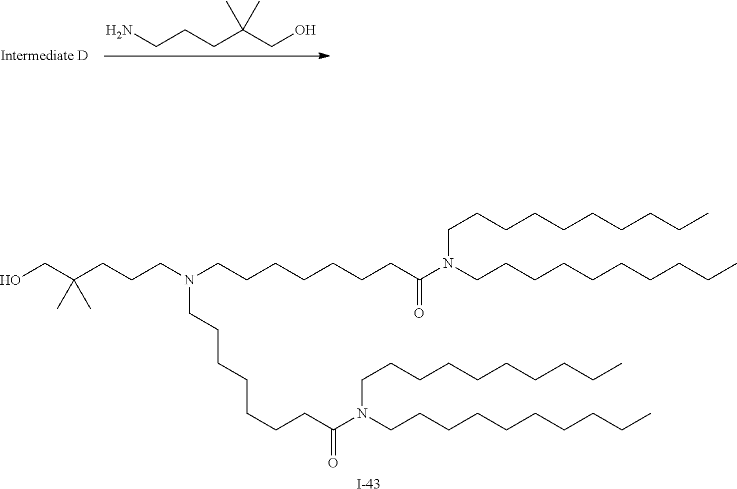

### Synthesis of I-43

A mixture of Intermediate D (1.2 mmol, 600 mg), 5-amino-4,4-dimethylpentan-1-ol (0.74 mmol, 97 mg), and DIEA (3.0 mmol, 0.52 mL) in ACN (7 mL) was heated at 72° C. for 48 h. The reaction mixture was concentrated and the crude material was purified via automated flash chromatography (5% to 60% EtOAc in hexanes with 1% Et₃N) to give compound I-43 (280 mg, 48%). ¹H NMR (400 MHz, CDCl₃) δ 3.31-3.23 (m, 6H), 3.22-3.12 (m, 4H), 2.41-2.30 (m, 6H), 2.30-2.22 (m, 4H), 1.71-1.58 (m, 4H), 1.56-1.46 (m, 5H), 1.44-1.36 (m, 3H), 1.36-1.17 (t, 74H), 0.98-0.81 (m, 18H). ESI-MS: MW for C₆₃H₁₂₇N₃O₃ [M+H]⁺ Calc. 975.0; Found 975.4.

### Example 43

### 8,8′-((3-(2-Methyl-1H-Imidazol--Yl)Propyl)Azanediyl)Bis(N,N-Didecyloctanamide) (Compound I-44)

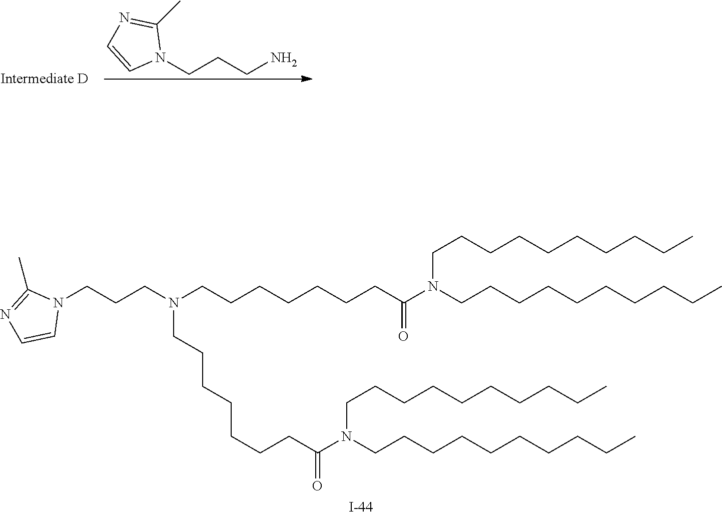

### Synthesis of I-44

A mixture of Intermediate D (1.2 mmol, 600 mg), 3-(2-methyl-1H-imidazol-1-yl)propan-1-amine (0.74 mmol, 103 mg), and DIEA (3.0 mmol, 0.52 mL) in ACN (7 mL) was heated at 72° C. for 48 h. The reaction mixture was concentrated and the crude material was purified via automated flash chromatography (5% to 100% EtOAc in hexanes with 1% Et₃N). A second purification via automated flash chromatography (c18 column, 50% to 100% MeOH in water with 1% TFA) gave pure compound I-44 (60 mg, 10%). ¹H NMR (400 MHz, CDCl₃) δ 6.90 (s, 1H), 6.82 (s, 1H), 3.87 (t, J=7.2 Hz, 2H), 3.34-3.24 (m, 4H), 3.23-3.11 (m, 4H), 2.43-2.34 (m, 8H), 2.31-2.23 (m, 4H), 1.89-1.77 (m, 3H), 1.67-1.59 (m, 4H), 1.58-1.45 (m, 8H), 1.45-1.18 (m, 72H), 0.94-0.83 (m, 12H). ESI-MS: MW for C₆₃H₁₂₃N₃O₂ [M+H]⁺ Calc. 983.0; Found 983.4.

### Example 44

### 8,8′-((7-Hydroxyheptyl)Azanediyl)Bis(N,N-Didecyloctanamide) (Compound I-45)

Compound I-45 was prepared according to the general procedures of example 13 to yield the desired product (320 mg, 55%). ¹HNMR (400 MHz, CDCl₃ at 7.26 ppm) δ: 6.90 (s, 1H), 6.82 (s, 1H), 3.87 (t, J=7.2 Hz, 2H), 3.33-3.22 (m, 4H), 3.23-3.13 (m, 4H), 2.42-2.34 (m, 8H), 2.33-2.20 (m, 4H), 1.90-1.76 (m, 2H), 1.66-1.59 (m, 4H), 1.57-1.45 (m, 8H), 1.45-1.13 (m, 71H), 0.88 (m, 12H). ESI-MS: MW for C₆₃H₁₂₇N₃O₃ [M+H]⁺ Calc. 975.0; Found 975.4.

### Example 45

### 8,8′-((2-(2-Methoxyethoxy)Ethyl)Azanediyl)Bis(N,N-Didecyloctanamide) (Compound I-46)

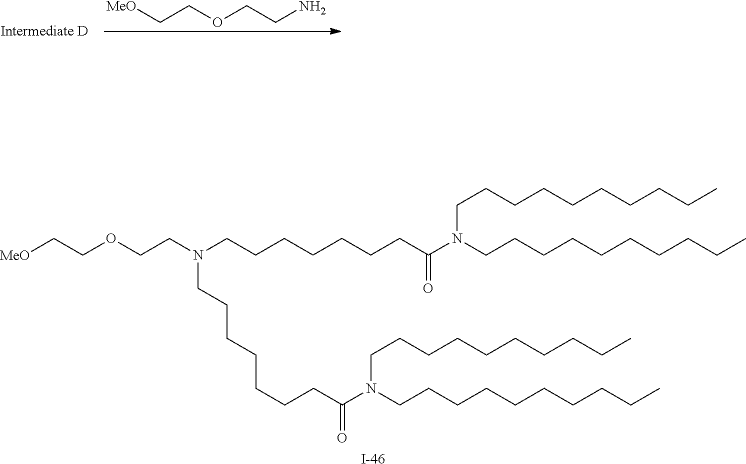

### Synthesis of I-46

A mixture of Intermediate D (1.2 mmol, 600 mg), 2-(2-methoxyethoxy)ethan-1-amine (0.74 mmol, 89 mg), and DIEA (3.0 mmol, 0.52 mL) in ACN (7 mL) was heated at 72° C. for 48 h. The reaction mixture was concentrated and the crude material was purified via automated flash chromatography (5% to 60% EtOAc in hexanes with 1% Et₃N) to give compound I-46 (333 mg, 58%). ¹H NMR (400 MHz, CDCl₃) δ 3.65-3.60 (m, 2H), 3.59-3.51 (m, 4H), 3.40 (s, 3H), 3.33-3.26 (m, 4H), 3.25-3.18 (m, 4H), 2.67 (t, J=6.7 Hz, 2H), 2.48-2.39 (m, 4H), 2.33-2.25 (m, 4H), 1.68-1.61 (m, 4H), 1.59-1.48 (m, 8H), 1.48-1.40 (m, 3H), 1.37-1.24 (m, 69H), 0.95-0.85 (m, 12H). ESI-MS: MW for C₆₁H₁₂₃N₃O₄ [M+H]⁺ Calc. 963.0; Found 963.4.

### Example 46

### 8,8′-((8-Hydroxyoctyl)Azanediyl)Bis(N,N-Didecyloctanamide) (Compound I-47)

Compound I-47 was prepared according to the general procedures of example 13 to yield the desired product (320 mg, 54%). ¹HNMR (600 MHz, CDCl₃ at 7.26 ppm) δ: 3.65 (t, J=6.7 Hz, 2H), 3.32-3.27 (m, 4H), 3.23-3.18 (m, 4H), 2.41-2.34 (m, 6H), 2.32-2.25 (m, 4H), 1.68-1.62 (m, 4H), 1.60-1.48 (m, 10H), 1.47-1.39 (m, 6H), 1.38-1.23 (m, 76H), 0.96-0.84 (m, 12H). ESI-MS: MW for C₆₄H₁₂₉N₃O₃ [M+H]⁺ Calc. 989.0; Found 989.5.

### Example 47

### 8,8′-((3-(1H-Imidazol-1-Yl)Propyl)Azanediyl)Bis(N,N-Didecyloctanamide) (Compound I-48)

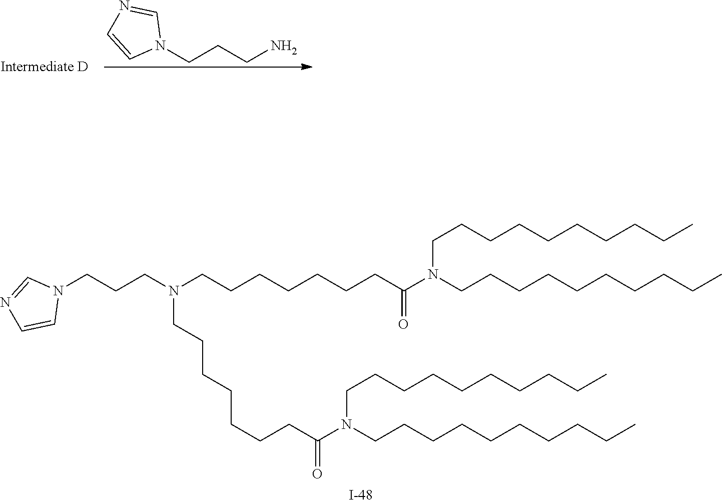

### Synthesis of I-48

A mixture of Intermediate D (1.2 mmol, 600 mg), 3-(1H-imidazol-1-yl)propan-1-amine (0.74 mmol, 93 mg), and DIEA (3.0 mmol, 0.52 mL) in ACN (7 mL) was heated at 72° C. for 48 h. The reaction mixture was concentrated and the crude material was purified via automated flash chromatography (5% to 100% EtOAc in hexanes with 1% Et₃N) to give compound I-48 (82 mg, 14%). ¹H NMR (600 MHz, CDCl₃) δ 7.48 (s, 1H), 7.07 (s, 1H), 6.93 (s, 1H), 4.00 (t, J=7.0 Hz, 2H), 3.30 (t, J=7.7 Hz, 4H), 3.21 (t, J=7.8 Hz, 4H), 2.41-2.34 (m, 6H), 2.29 (t, J=7.6 Hz, 4H), 1.92-1.87 (m, 2H), 1.69-1.61 (m, 4H), 1.59-1.47 (m, 8H), 1.41-1.22 (m, 74H), 0.93-0.87 (m, 12H). ESI-MS: MW for C₆₂H₁₂₁N₅O₂ [M+H]⁺ Calc. 969.0; Found 969.1.

### Example 48

### 8,8′-((2,2-Difluoro-3-Hydroxypropyl)Azanediyl)Bis(N,N-Didecyloctanamide) (Compound I-49)

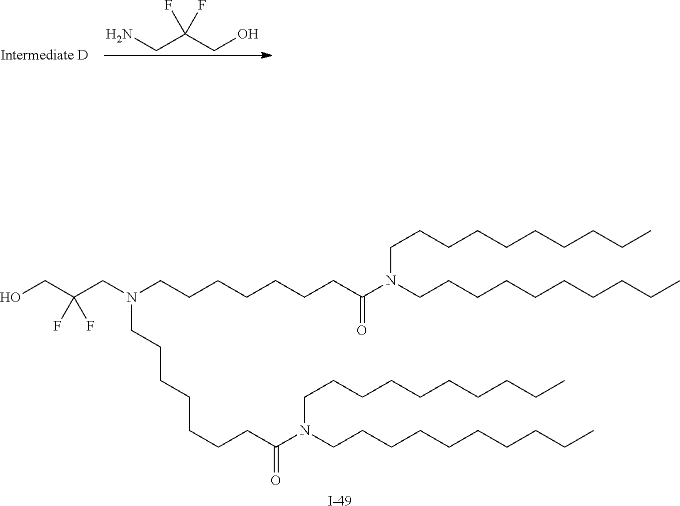

### Synthesis of I-49

A mixture of Intermediate D (1.0 mmol, 500 mg), 3-amino-2,2-difluoropropan-1-ol (0.62 mmol, 69 mg), and DIEA (3.0 mmol, 0.43 mL) in ACN (6 mL) was heated at 72° C. for 48 h. The reaction mixture was concentrated and the crude material was purified via automated flash chromatography (5% to 60% EtOAc in hexanes with 1% Et₃N) to give compound I-49 (55 mg, 12%). ¹H NMR (600 MHz, CDCl₃) δ 4.87 (s, 1H), 3.88 (t, J=12.1 Hz, 2H), 3.33-3.27 (m, 4H), 3.24-3.18 (m, 4H), 2.96 (t, J=12.7 Hz, 2H), 2.56-2.51 (m, 4H), 2.32-2.26 (m, 4H), 1.68-1.61 (m, 6H), 1.59-1.42 (m, 12H), 1.39-1.23 (m, 70H), 0.93-0.87 (m, 12H). ESI-MS: MW for C₅₉H₁₁₇F₂N₃O₃[M+H]⁺ Calc. 954.9; Found 955.1.

### Example 49

### 8,8′-((3-((2-(Methylamino)-3,4-Dioxocyclobut-1-En-Yl)Amino)Propyl)Azanediyl)Bis(N,N-Didecyloctanamide) (Compound 1-50)

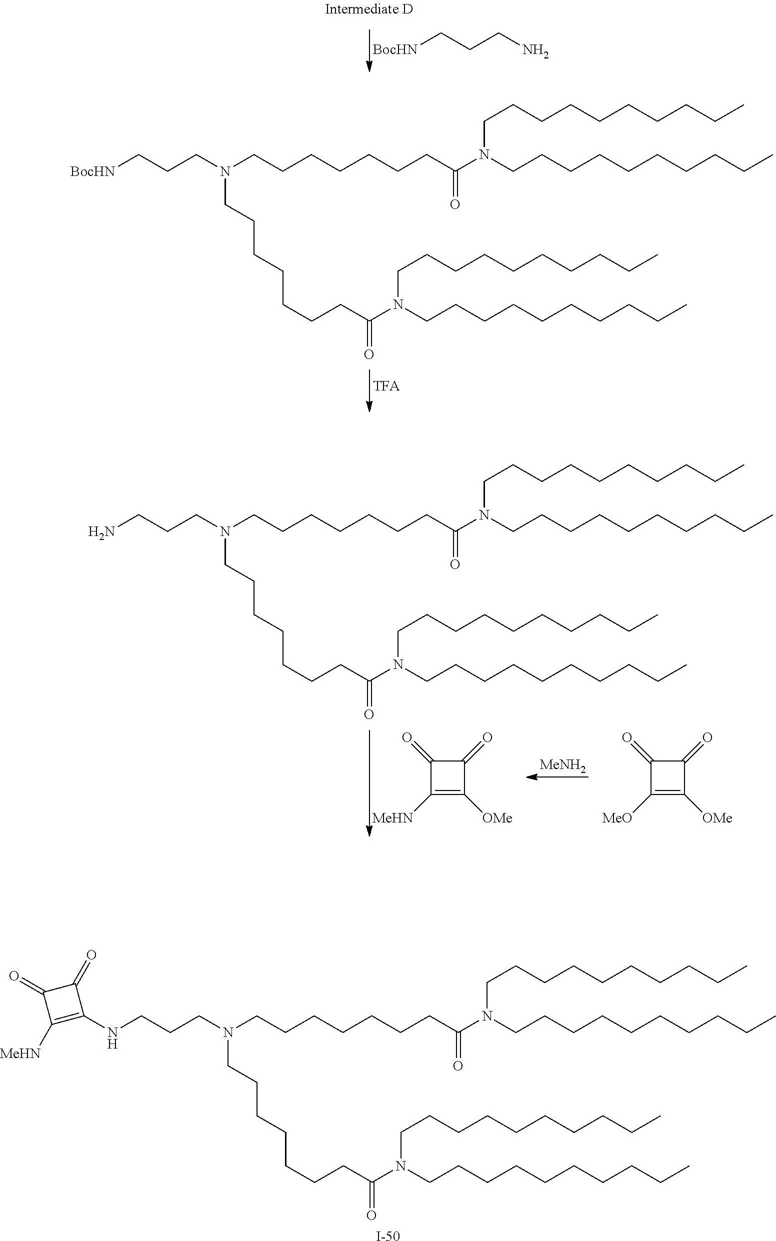

### Synthesis of tert-butyl (3-(bis(8-(didecylamino)-8-oxooctyl)amino)propyl)carbamate

A mixture of Intermediate D (1.60 mmol, 800 mg), tert-butyl (3-aminopropyl)carbamate (0.99 mmol, 173 mg), and DIEA (4.0 mmol, 0.7 mL) in ACN (10 mL) was heated at 72° C. for 48 h. The reaction mixture was concentrated and the crude material was purified via automated flash chromatography (5% to 60% EtOAc in hexanes with 1% Et₃N) to give compound tert-butyl (3-(bis(8-(didecylamino)-8-oxooctyl)amino)propyl)carbamate (388 mg, 48%).

### Synthesis of 8,8′-((3-aminopropyl)azanediyl)bis(N,N-didecyloctanamide)

A mixture of tert-butyl (3-(bis(8-(didecylamino)-8-oxooctyl)amino)propyl)carbamate (0.38 mmol, 385 mg) and TFA (1.0 mL) in DCM (2.0 mL) was stirred at room temperature for 90 min. The reaction mixture was concentrated and the crude material was partitioned between EtOAc and sat. NaHCO₃ to give compound 8,8′-((3-aminopropyl)azanediyl)bis(N,N-didecyloctanamide) (385 mg, quantitative) which was used in the next step without further purification.

### Synthesis of 3-methoxy-4-(methylamino)cyclobut-3-ene-1,2-dione

A mixture of 3,4-dimethoxycyclobut-3-ene-1,2-dione (7.0 mmol, 1 g) and 2M methylamine in THF (7.7 mmol, 3.9 mL) in diethyl ether (100 mL) was stirred at room temperature for 19 h. The solids were filtered and washed with diethyl ether. The crude solids were triturated in hot EtOAc, cooled to 5° C., then filtered and washed with cold EtOAc to give 3-methoxy-4-(methylamino)cyclobut-3-ene-1,2-dione (700 mg, 71%).

### Synthesis of I-50

A mixture of 8,8′-((3-aminopropyl)azanediyl)bis(N,N-didecyloctanamide) (0.11 mmol, 100 mg) and 3-methoxy-4-(methylamino)cyclobut-3-ene-1,2-dione (0.11 mmol, 15 mg) in ethanol (5 mL) was stirred at room temperature for 19 h then heated at 50° C. for 19 h. The reaction mixture was concentrated and the crude material was purified via automated flash chromatography (5% to 100% EtOAc in hexanes with 1% Et₃N, then 2% MeOH in DCM). A second purification via automated flash chromatography (1% to 10% MeOH in DCM) was performed to give I-50 (32 mg, 28%). ¹H NMR (400 MHz, CDCl₃) δ 8.22 (bs, 0.5H), 7.60 (bs, 0.5H), 3.70 (bs, 2H), 3.33-3.23 (m, 7H), 3.23-3.14 (m, 4H), 2.96 (bs, 2H), 2.80 (bs, 4H), 2.28 (t, J=7.4 Hz, 4H), 1.98 (bs, 2H), 1.66-1.45 (m, 16H), 1.40-1.20 (m, 67H). 0.92-0.83 (m, 12H). ESI-MS: MW for C₆₄H₁₂₃N₅O₄ [M+H]⁺ Calc. 1027.0; Found 1027.1.

### Example 50

### 8,8′-((2-Fluoro-3-Hydroxypropyl)Azanediyl)Bis(N,N-Didecyloctanamide) (Compound I-51)

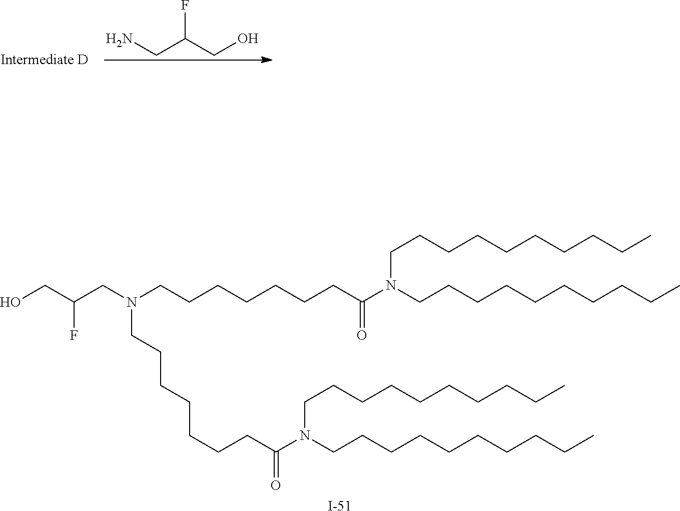

### Synthesis of I-51

A mixture of Intermediate D (1.0 mmol, 500 mg), 3-amino-2-fluoropropan-1-ol hydrochloride (0.62 mmol, 80 mg), and DIEA (3.1 mmol, 0.54 mL) in ACN (6 mL) was heated at 72° C. for 24 h, then at 55° C. for 72 h. The reaction mixture was concentrated and the crude material was purified via automated flash chromatography (5% to 60% EtOAc in hexanes with 1% Et₃N) to give compound I-51 (45 mg, 10%). ¹H NMR (400 MHz, CDCl₃) δ 4.60 (dt, J=46.8, 4.8 Hz, 1H), 3.92-3.80 (m, 2H), 3.35-3.24 (m, 4H), 3.24-3.12 (m, 4H), 2.85-2.72 (m, 2H), 2.53-2.37 (m, 4H), 2.31-2.23 (m, 4H), 1.67-1.40 (m, 20H), 1.38-1.19 (m, 66H), 0.94-0.82 (m, 12H). ESI-MS: MW for C₅₉H₁₁₈FN₃O₃[M+H]⁺ Calc. 936.9; Found 937.0.

### Example 51

### 8,8′-((3,3,3-Trifluoro-2-(Hydroxymethyl)Propyl)Azanediyl)Bis(N,N-Didecyloctanamide) (Compound I-52)

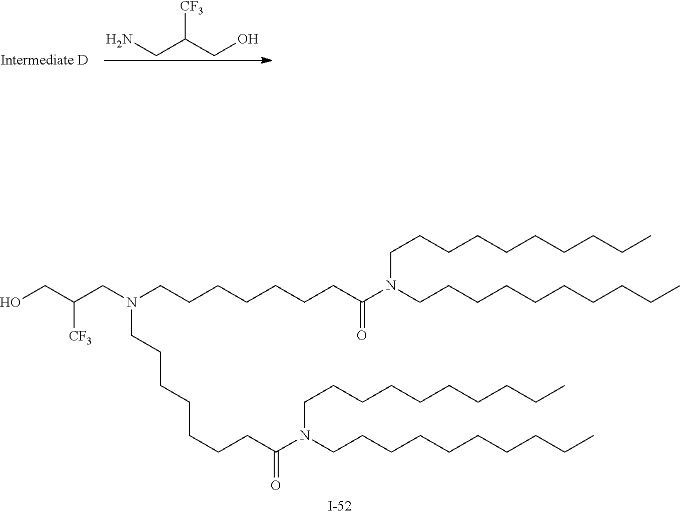

### Synthesis of I-52

A mixture of Intermediate D (1.0 mmol, 500 mg), 2-(aminomethyl)-3,3,3-trifluoropropan-1-ol (0.62 mmol, 111 mg), and DIEA (2.4 mmol, 0.43 mL) in ACN (6 mL) was heated at 72° C. for 24 h, then at 55° C. for 72 h. The reaction mixture was concentrated and the crude material was purified via automated flash chromatography (5% to 60% EtOAc in hexanes with 1% Et₃N) to give compound I-52 (100 mg, 20%). ¹H NMR (400 MHz, CDCl₃) δ 5.88 (s, 1H), 4.01-3.92 (m, 1H), 3.89-3.78 (m, 1H), 3.33-3.24 (m, 4H), 3.23-3.15 (m, 4H), 2.91-2.73 (m, 2H), 2.69-2.50 (m, 3H), 2.34-2.21 (m, 6H), 1.69-1.39 (m, 20H), 1.38-1.21 (m, 68H), 0.95-0.84 (m, 12H). ESI-MS: MW for C₆₀H₁₁₈F₃N₃O₃[M+H]⁺ Calc. 986.9; Found 987.0.

### Example 52

### 8,8′-((5-Methoxypentyl)Azanediyl)Bis(N,N-Didecyloctanamide) (Compound I-53)

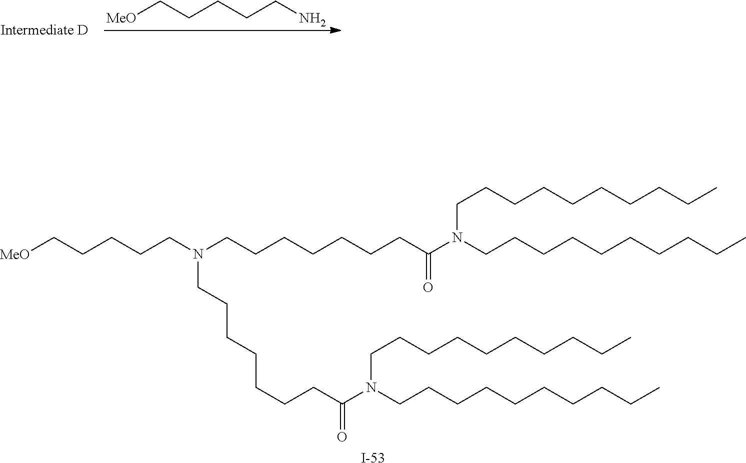

### Synthesis of I-53

A mixture of Intermediate D (0.80 mmol, 400 mg), 5-methoxypentan-1-amine (0.50 mmol, 58 mg), and DIEA (2.0 mmol, 0.35 mL) in ACN (6 mL) was heated at 72° C. for 24 h, then at 55° C. for 72 h. The reaction mixture was concentrated and the crude material was purified via automated flash chromatography (5% to 60% EtOAc in hexanes with 1% Et₃N) to give compound I-53 (210 mg, 55%). ¹H NMR (400 MHz, CDCl₃) δ 3.36 (t, J=6.6 Hz, 2H), 3.32 (s, 3H), 3.30-3.25 (m, 4H), 3.22-3.16 (m, 4H), 2.41-2.32 (m, 6H), 2.31-2.22 (m, 4H), 1.67-1.37 (m, 23H), 1.36-1.19 (m, 69H), 0.91-0.85 (m, 12H). ESI-MS: MW for C₆₂H₁₂₅N₃O₃ [M+H]⁺ Calc. 961.0; Found 961.1.

### Example 53

### N,N-Didecyl-8-((8-(Dioctylamino)-8-Oxooctyl)(Methyl)Amino)Octanamide (Compound I-55)

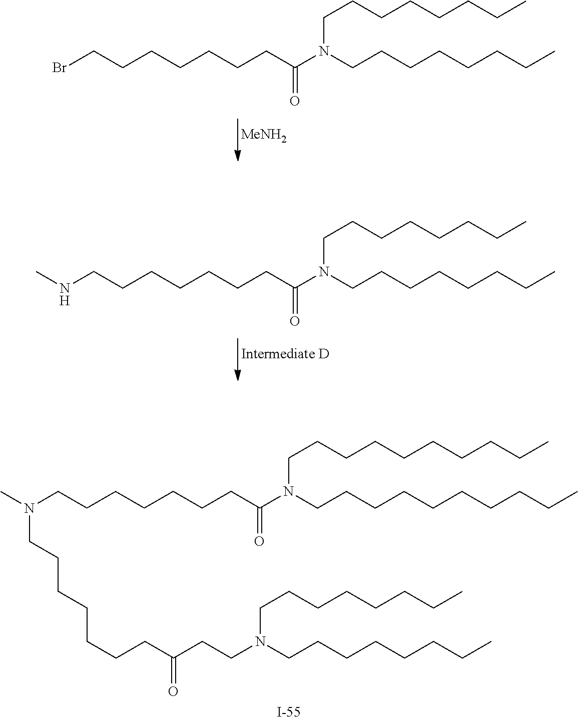

### Synthesis of 8-(methylamino)-N,N-dioctyloctanamide

A mixture of 8-bromo-N,N-dioctyloctanamide (5.8 mmol, 2.6 g) and 8M methylamine in EtOH (30 mL) in ACN (12 mL) was heated at 70° C. for 19 h. The reaction mixture was concentrated and the crude material was purified via automated flash chromatography (5% to 100% EtOAc in hexanes, then 1% to 10% MeOH in DCM) to give compound 8-(methylamino)-N,N-dioctyloctanamide (1.91 g, 83%).

### Synthesis of I-55

A mixture of 8-(methylamino)-N,N-dioctyloctanamide (3.4 mmol, 1.3 g), Intermediate D (3.4 mmol, 1.7 g), and DIEA (13.8 mmol, 2.4 mL) in ACN (120 mL) was heated at 70° C. for 19 h. The reaction mixture was concentrated and the crude material was purified via automated flash chromatography (5% to 60% EtOAc in hexanes with 1% Et₃N) to give compound I-55 (1.38 g, 50%). ¹H NMR (400 MHz, CDCl₃) δ 3.34-3.26 (m, 4H), 3.25-3.17 (m, 4H), 2.34-2.25 (m, 8H), 2.21 (s, 3H), 1.70-1.61 (m, 6H), 1.60-1.41 (m, 12H), 1.39-1.23 (m, 59H), 0.95-0.85 (m, 12H). ESI-MS: MW for C₅₃H₁₀₇N₃O₂ [M+H]⁺ Calc. 818.8; Found 819.0.

### Example 54

### Tert-Butyl (3-(Bis(10-(Didecylamino)-10-Oxodecyl)Amino)Propyl)Carbamate (Compound I-56)

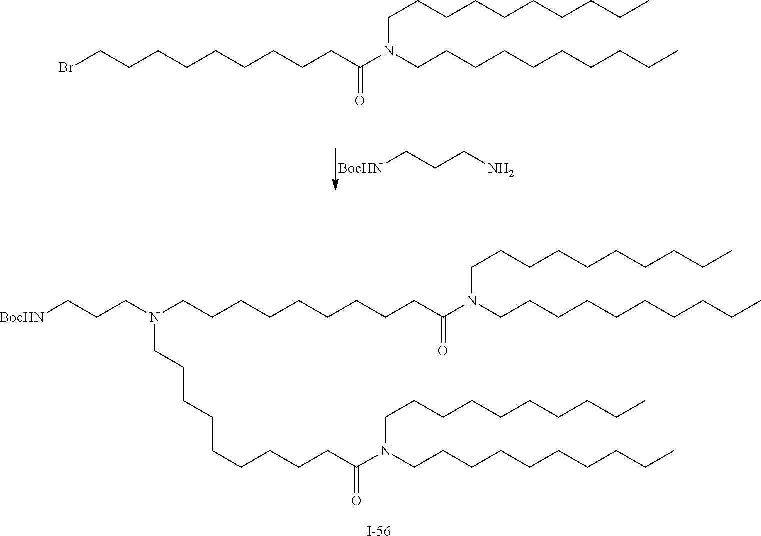

### Synthesis of I-56

To mixture of 10-bromo-N,N-didecyldecanamide (was prepared according to the general procedures of example 5, 1.5 mmol, 800 mg), tert-butyl (3-aminopropyl)carbamate (0.93 mmol, 162 mg), and DIEA (4.5 mmol, 0.78 mL), in ACN (10 mL) was stirred at 70° C. for 48 h. The reaction mixture was concentrated and the crude material was purified via automated flash chromatography (5% to 65% EtOAc in hexanes with 1% Et₃N) to give I-56 (555 mg, 69%). ¹H NMR (400 MHz, CDCl₃) δ 5.72 (bs, 1H), 3.32-3.24 (m, 4H), 3.23-3.14 (m, 6H), 2.44 (t, J=6.4 Hz, 2H), 2.31-2.23 (m, 4H), 2.31-2.23 (m, 4H), 1.67-1.58 (m, 9H), 1.57-1.47 (m, 3H), 1.47-1.37 (m, 12H), 1.35-1.19 (m, 77H), 0.92-0.83 (m, 12H). ESI-MS: MW for C₆₈H₁₃₆N₄O₄ [M+H]⁺ Calc. 1074.1; Found 1074.2.

### Example 55

### 10,10′-((3-(1H-Imidazol-1-Yl)Propyl)Azanediyl)Bis(N,N-Didecyldecanamide) (Compound I-57)

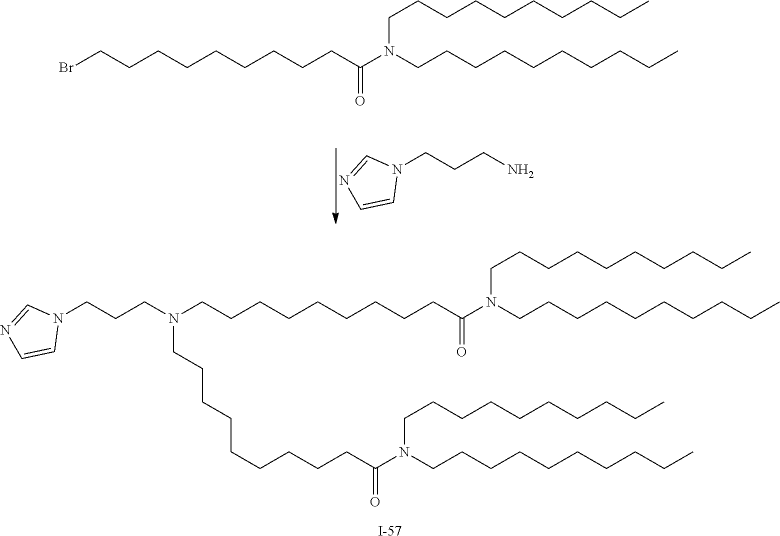

### Synthesis of I-57

A mixture of 10-bromo-N,N-didecyldecanamide (was prepared according to the general procedures of example 5, 1.1 mmol, 600 mg), 3-(1H-imidazol-1-yl)propan-1-amine (0.68 mmol, 88 mg), and DIEA (3.3 mmol, 0.57 mL) in ACN (7 mL) was heated at 70° C. for 48 h. The reaction mixture was concentrated and the crude material was purified via automated flash chromatography (5% to 100% EtOAc in hexanes with 1% Et₃N). A second purification via reverse phase (C₁₈) automated flash chromatography (50% to 100% MeOH in water) gave compound I-57 (45 mg, 8%). ¹H NMR (400 MHz, CDCl₃) δ 7.52 (bs, 1H), 7.06 (s, 1H), 6.92 (s, 1H), 4.00 (t, J=7.0 Hz, 2H), 3.32-3.24 (m, 4H), 3.22-3.15 (m, 4H), 2.38 (bs, 6H), 2.31-2.21 (m, 4H), 1.90 (bs, 2H), 1.73-1.57 (m, 8H), 1.57-1.45 (m, 8H), 1.43-1.16 (m, 81H), 0.95-0.81 (m, 12H). ESI-MS: MW for C₆₆H₁₂₉N₅O₂ [M+H]⁺ Calc. 1025.0; Found 1025.1.

### Example 56

### 8,8′-(Methylazanediyl)Bis(N,N-Dinonyloctanamide) (Compound I-58)

Compound I-58 was prepared according to the general procedures of example 5 to yield the desired product (1.78 g, 59%). ¹H NMR (600 MHz, CDCl₃) δ 3.33-3.27 (m, 4H), 3.24-3.18 (m, 4H), 2.33-2.26 (m, 8H), 2.21 (s, 3H), 1.69-1.61 (m, 7H), 1.59-1.42 (m, 12H), 1.39-1.21 (m, 61H), 0.94-0.86 (m, 12H). ESI-MS: MW for C₅₃H₁₀₇N₃O₂ [M+H]⁺ Calc. 818.8; Found 819.0.

### Example 57

### Tert-Butyl (3-(Bis(10-(Didecylamino)-10-Oxodecyl)Amino)Propyl)Carbamate (Compound I-59)

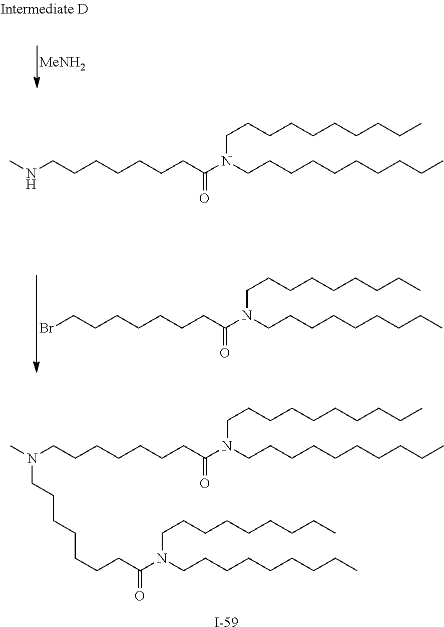

### Synthesis of N,N-didecyl-8-(methylamino)octanamide

A mixture of Intermediate D (6.3 mmol, 2.8 g) and 8M methylamine in EtOH (30 mL) in ACN (12 mL) was heated at 70° C. for 19 h. The reaction mixture was concentrated and the crude material was purified via automated flash chromatography (5% to 100% EtOAc in hexanes, then 1% to 10% MeOH in DCM) to give N,N-didecyl-8-(methylamino)octanamide (2.24 g, 79%).

### Synthesis of I-59

A mixture of N,N-didecyl-8-(methylamino)octanamide (3.3 mmol, 1.5 g), 8-bromo-N,N-dinonyloctanamide (was prepared according to the general procedures of example 5, 3.3 mmol, 1.6 g), and DIEA (13.2 mmol, 2.3 mL) in ACN (10 mL) was heated at 70° C. for 19 h. The reaction mixture was concentrated and the crude material was purified via automated flash chromatography (5% to 60% EtOAc in hexanes with 1% Et₃N) to give compound I-59 (1.5 g, 53%). ¹H NMR (400 MHz, CDCl₃) δ 3.34-3.26 (m, 4H), 3.25-3.17 (m, 4H), 2.34-2.24 (m, 8H), 2.21 (s, 3H), 1.75-1.59 (m, 4H), 1.61-1.41 (m, 12H), 1.38-1.23 (m, 66H), 0.95-0.86 (m, 12H). ESI-MS: MW for C₅₅H₁₁₁N₃O₂[M+H]⁺ Calc. 846.9; Found 847.1.

### Example 58

### 10,10′-((3-((2-(Methylamino)-3,4-Dioxocyclobut-1-En-1-Yl)Amino)Propyl)Azanediyl)Bis(N,N-Didecyldecanamide) (Compound I-60)

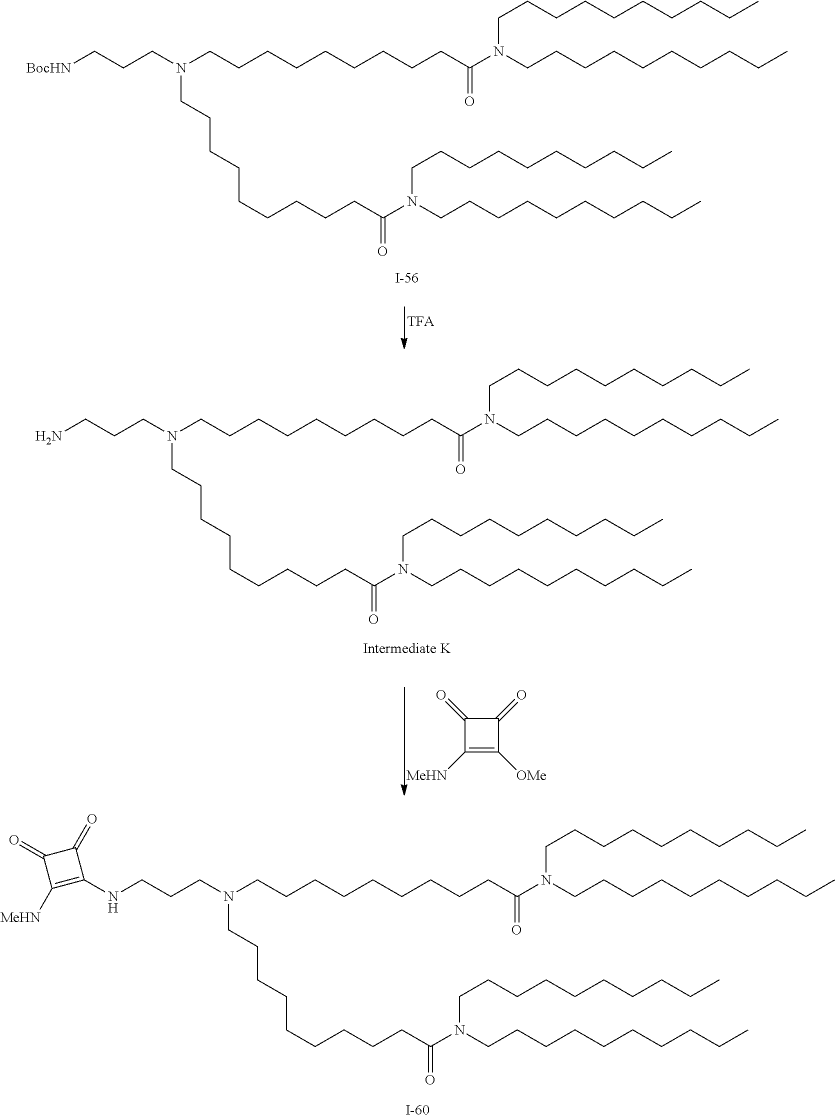

### Synthesis of Intermediate K

A mixture of I-56 (0.42 mmol, 455 mg) and TFA (2.0 mL) in DCM (1.0 mL) was stirred at room temperature for 2 h. The reaction mixture was concentrated and the crude material was partitioned between EtOAc and sat. NaHCO₃ to give 10,10′-((3-aminopropyl)azanediyl)bis(N,N-didecyldecanamide) (Intermediate K, 369 mg, 90%) which was used in the next step without further purification.

### Synthesis of I-60

A mixture of Intermediate K (0.1 mmol, 100 mg) and 3-methoxy-4-(methylamino)cyclobut-3-ene-1,2-dione (was prepared according to the general procedures of example 49, 0.5 mmol, 75 mg) in EtOH (1.0 mL) was heated at 80° C. for 5 h. The reaction mixture was concentrated and the crude material was purified via automated flash chromatography (50% to 100% EtOAc in hexanes with 1% Et₃N, then 2% to 10% MeOH in DCM) to give I-60 (27 mg, 25%). ¹H NMR (400 MHz, CDCl₃) δ 7.15-6.86 (bs, 1H), 3.68 (bs, 2H), 3.33-3.18 (m, 11H), 2.58 (t, J=6.7 Hz, 2H), 2.46 (t, J=7.6 Hz, 4H), 2.30 (t, J=7.6 Hz, 4H), 2.05-1.73 (m, 6H), 1.71-1.41 (m, 18H), 1.38-1.20 (m, 79H), 0.96-0.83 (m, 12H). ESI-MS: MW for C₆₈H₁₃₁N₅O₄ [M+H]⁺ Calc. 1083.0; Found 1083.1.

### Example 59

### Synthetic Pathway for Compounds I-61 to I-68

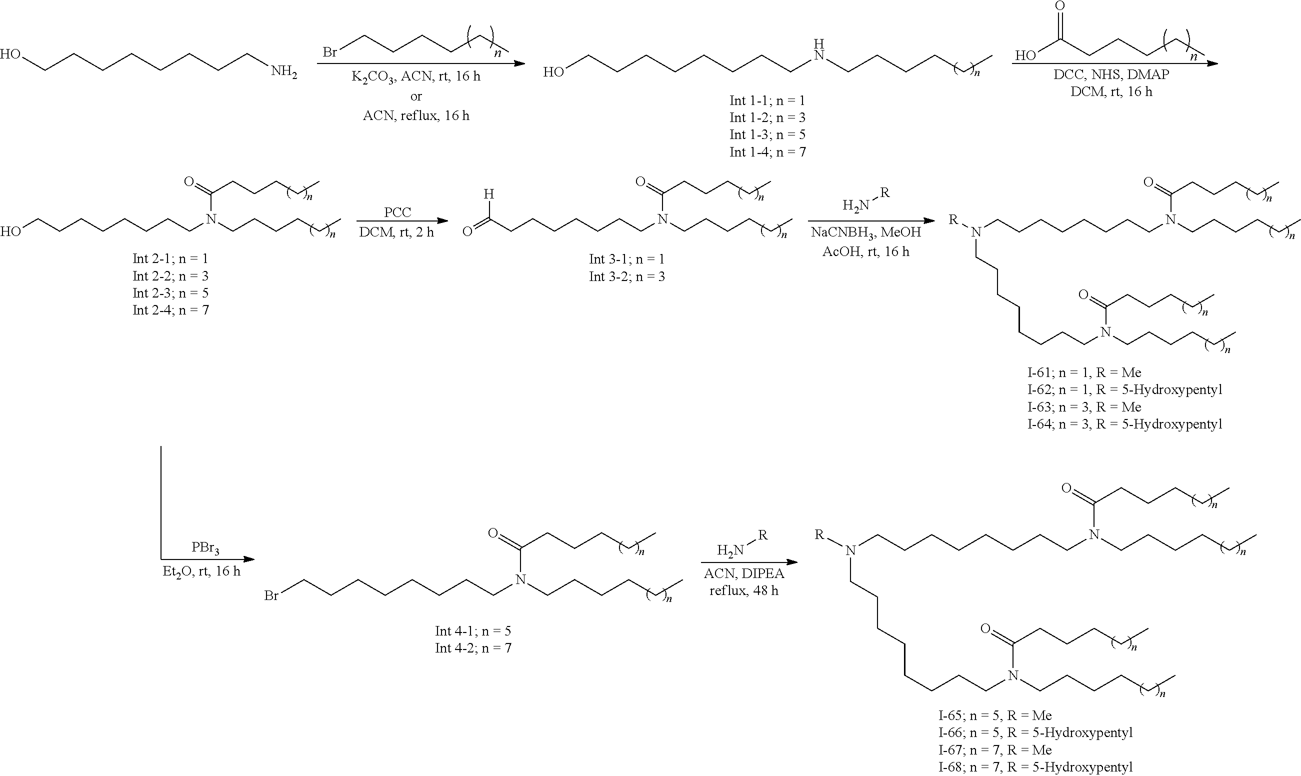

### General Procedure for the Preparation of the Int 1-1 & Int 1-2

To a solution of K₂CO₃ (1.0 eq) in ACN (7 mL/mmol) was added 8-aminooctanol (1.0 eq) at RT under N₂, and the resulting suspension was stirred for 30 min at RT. Appropriate bromide (1.0 eq) was then added dropwise and the reaction mixture was stirred overnight at RT under N₂. Then, the suspension was filtered, and the remaining solution was concentrated. The solid crude product was purified by column chromatography (DCM/3% NH₃ in MeOH, 100:0 to 80:20).

### General Procedure for the Preparation of the Int 1-3 & Int 1-4

To a solution of 8-aminooctanol (1.0 eq) in ACN (7 mL/mmol) was added appropriate bromide (1.0 eq) and the reaction mixture was refluxed overnight under N₂. Then, the reaction mixture was concentrated to afford the crude product. The solid crude product was purified by column chromatography (DCM/3% NH₃ in MeOH, 100:0 to 80:20).

### General Procedure or the Preparation of the Int 2-1 to Int 2-4

To a solution of acid (0.9 eq) in DCM (2.5 mL/mmol) were added N-hydroxysuccinimide (0.9 eq), 4-dimethylaminopyridine (0.9 eq) and dicyclohexylcarbodiimide (0.9 eq) at RT under N₂. The resulting mixture was stirred at RT until complete conversion. The precipitate was filtered off, and the filtrate was added dropwise to a solution of the Int 1-1 or Int 1-2, Int 1-3, or Int 1-4 (1.0 eq) in DCM (2.3 mL/mmol) at RT. The reaction mixture was stirred overnight at RT under N₂. Then, the organic phase was washed with HCl (aq., 1 mol/L), Na₂CO₃ (aq.), and dried over Na₂SO₄. The organic phase was concentrated. The pale-yellow solid was purified by column chromatography (Hex/EtOAc, 100:0 to 0:100).

### General Procedure for the Preparation of the Int 3-1 & Int 3-2

To a solution of the Int 2-1 or Int 2-2 (1.0 eq) in DCM (10-15 mL/mmol) was added PCC (5.0 eq) in small portions. The resulting suspension was stirred at RT for two hours, under N₂. Then, the mixture was concentrated under reduced pressure, and the obtained crude product was purified by chromatography (Hex/EtOAc, 100:0 to 80:20).

### General Procedure for the Preparation of the Int 4-1 & Int 4-2

To a solution of Int 2-3 or Int 2-4 (1.0 eq) in diethyl ether (8.0 mL/mmol) was added PBr₃ (2-3 eq) dropwise at 0° C. under N₂. The reaction mixture was slowly warmed up to RT and stirred at RT under N₂ until the completion of the reaction. Then, ice-cooled water was added gradually until a clear solution was obtained. The aqueous phase was extracted with ethyl acetate three times, dried over Na₂SO₄, and concentrated under reduced pressure. The crude product was purified by column chromatography (Hex/EtOAc, 100:0 to 90:10).

### General Procedure for the Preparation of the Compounds I-61, I-62, I-63 & I-64

To a solution of the Int 3-1 or Int 3-2 (1.0 eq) in MeOH (10 mL/mmol) was added the appropriate primary amine (0.35 eq). The resultant solution was stirred at RT for 30 min under N₂. Then, Na(CNBH₃) (4.0 eq), and a drop of AcOH were added and the reaction mixture was stirred for 2-3 d at RT under N₂. After the complete reaction, it was diluted with DCM (100 mL/mmol) and Na₂CO₃ (aq.) (200 mL/mmol), stirred for 30 min at RT, separated and the organic phase dried over Na₂SO₄. The crude product was purified by chromatography (DCM/3% NH₃ in MeOH, 100:0 to 80:20).

### General Procedure for the Preparation of the Compounds I-65, I-66, I-67 & I-68

To a solution of Int 4-1 or Int 4-2 (1.5 eq) in ACN (5.5 mL/mmol) were added DIPEA (3.8 eq.) and the desired alkylation reagent (1.0 eq). The reaction was carried out in a sealed tube at 80° C. for 24 h. Then, more Int 4-1 or Int 4-2 (0.5 eq) in ACN (2.0 mL/mmol) was added to the reaction mixture, and stirring continued for another 24 h at 80° C. The reaction mixture was then cooled to RT and concentrated under reduced pressure. The crude product was purified by column chromatography (Hex/1% NEt₃ in EtOAc, 95:5 to 0:100).

### Synthesis of N,N′-((methylazanediyl)bis(octane-8,1-diyl))bis(N-hexylhexanamide) (I-61)

N,N′-((methylazanediyl)bis(octane-8,1-diyl))bis(N-hexylhexanamide) (I-61) was prepared according to the general procedure from Int 3-1 (500 mg, 1.5 mmol) and methylamine (2 M in MeOH, 0.26 mL, 0.52 mmol). The product was obtained as pale-yellow oil (250 mg, 0.38 mmol, 73%). ¹H NMR (400 MHz, CDCl₃) δ 3.28 (dd, J=7.8, 7.4 Hz, 4H), 3.19 (dd, J=7.5 Hz, 4H), 2.34 (s, 3H), 2.30-2.17 (m, 7H), 1.63 (dt, J=14.8, 7.6 Hz, 6H), 1.52 (td, J=14.4, 7.4 Hz, 12H), 1.39-1.19 (m, 40H), 0.92-0.87 (m, 12H)., ESI-MS: MW for C₄₁H₈₃N₃O₂ [M+H]⁺ calc. 650.66, found 650.85.

### Synthesis of N,N′-(((5-hydroxypentyl)azanediyl)bis(octane-8,1-diyl))bis(N-hexylhexanamide) (I-62)

N,N′-(((5-hydroxypentyl)azanediyl)bis(octane-8,1-diyl))bis(N-hexylhexanamide) (I-62) was prepared according to the general procedure from Int 3-1 (500 mg, 1.5 mmol) and 5-aminopentan-1-ol (54 mg, 0.52 mmol). The product was obtained as pale-yellow oil (242 mg, 0.34 mmol, 66%). ¹H NMR (400 MHz, CDCl₃) δ 3.64 (t, J=6.5 Hz, 2H), 3.27 (dd, J=10.8, 4.4 Hz, 4H), 3.22-3.16 (m, 4H), 2.45 (s, 1H), 2.29-2.24 (m, 4H), 1.81 (s, 1H), 1.62 (dt, J=16.1, 7.7 Hz, 7H), 1.50 (dd, J=14.4, 7.2 Hz, 15H), 1.40-1.19 (m, 42H), 0.93-0.85 (m, 12H), ESI-MS: MW for C₄₅H₉₁N₃O₃ [M+H]⁺ calc. 722.72, found 722.80.

### Synthesis of N,N′-((methylazanediyl)bis(octane-8,1-diyl))bis(N-octyloctanamide) (I-63)

N,N′-((methylazanediyl)bis(octane-8,1-diyl))bis(N-octyloctanamide) (I-63) was prepared according to the general procedure from Int 3-2 (450 mg, 1.2 mmol) and methylamine (2 M in MeOH, 0.21 mL, 0.42 mmol). The product was obtained as pale-yellow oil (180 mg, 0.24 mmol, 65%). ¹H NMR (400 MHz, CDCl₃) δ 3.30-3.24 (m, 4H), 3.22-3.14 (m, 4H), 2.63-2.59 (m, 3H), 2.47 (t, J=17.2 Hz, 3H), 2.30-2.23 (m, 4H), 1.75-1.57 (m, 8H), 1.57-1.42 (m, 9H), 1.29 (m, 52H), 0.91-0.84 (m, 12H), ESI-MS: MW for C₄₉H₉₉N₃O₂ [M+H]⁺ calc. 762.78, found 762.81.

### Synthesis of N,N′-(((5-hydroxypentyl)azanediyl)bis(octane-8,1-diyl))bis(N-octyloctanamide) (I-64)

N,N′-(((5-hydroxypentyl)azanediyl)bis(octane-8,1-diyl))bis(N-octyloctanamide) (I-64) was prepared according to the general procedure from Int 3-2 (450 mg, 1.2 mmol) and 5-aminopentan-1-ol (43.3 mg, 0.42 mmol). The product was obtained as pale-yellow oil (220 mg, 0.26 mmol, 66%). ¹H NMR (600 MHz, CDCl₃) δ 3.64 (t, J=6.5 Hz, 2H), 3.27 (dd, J=10.9, 4.4 Hz, 4H), 3.21-3.17 (m, 4H), 2.41 (s, 1H), 2.28-2.25 (m, 4H), 1.67-1.57 (m, 10H), 1.57-1.46 (m, 11H), 1.46-1.35 (m, 6H), 1.35-1.21 (m, 55H), 0.91-0.85 (m, 12H), ESI-MS: MW for C₅₃H₁₀₇N₃O₃ [M+H]⁺ calc. 834.84, found 834.70.

### Synthesis of N,N′-((methylazanediyl)bis(octane-8,1-diyl))bis(N-octyloctanamide) (I-65)

N,N′-((methylazanediyl)bis(octane-8,1-diyl))bis(N-octyloctanamide) (I-65) was prepared according to the general procedure from Int 4-1 (493 mg, 1.1 mmol) and methylamine (2 M in MeOH, 0.19 mL, 0.385 mmol). The product was obtained as pale-yellow oil (232 mg, 0.27 mmol, 66%). ¹H NMR (400 MHz, CDCl₃) δ 3.30-3.24 (m, 4H), 3.22-3.15 (m, 4H), 2.33-2.23 (m, 7H), 2.20 (s, 3H), 1.73-1.58 (m, 10H), 1.58-1.40 (m, 12H), 1.37-1.20 (m, 71H), 0.92-0.84 (m, 12H), ESI-MS: MW for C₅₇H₁₅N₃O₂ [M+H]⁺ calc. 874.91, found 875.60.

### Synthesis of N,N′-(((5-hydroxypentyl)azanediyl)bis(octane-8,1-diyl))bis(N-decyldecanamide) (I-66)

N,N′-(((5-hydroxypentyl)azanediyl)bis(octane-8,1-diyl))bis(N-decyldecanamide) (I-66) was prepared according to the general procedure from Int 4-1 (493 mg, 1.1 mmol) and 5-aminopentan-1-ol (40 mg, 0.385 mmol). The product was obtained as pale-yellow oil (247 mg, 0.26 mmol, 65%). The product was obtained as an oil (76.8 mg, 0.08 mmol, 28%). ¹H NMR (400 MHz, CDCl₃) δ 3.65 (t, J=6.4 Hz, 2H), 3.32-3.23 (m, 4H), 3.22-3.12 (m, 4H), 2.52 (s, 1H), 2.30-2.22 (m, 4H), 1.62 (dt, J=15.0, 7.5 Hz, 12H), 1.57-1.43 (m, 14H), 1.34-1.21 (m, 70H), 0.91-0.84 (m, 12H), ESI-MS: MW for C₆₁H₁₂₃N₃O₃ [M+H]⁺ calc. 946.97, found 946.73.

### Synthesis of N,N′-((methylazanediyl)bis(octane-8,1-diyl))bis(N-dodecyldodecanamide) (I-67)

N,N′-((methylazanediyl)bis(octane-8,1-diyl))bis(N-dodecyldodecanamide) (I-67) was prepared according to the general procedure from Int 4-2 (700 mg, 1.25 mmol) and methylamine (2 M in MeOH, 0.21 mL, 0.425 mmol). The product was obtained as pale-yellow oil (510 mg, 0.52 mmol, 84%). ¹H NMR (600 MHz, CDCl₃) δ 3.29-3.25 (m, 4H), 3.21-3.16 (m, 4H), 2.28 (dt, J=15.4, 8.4 Hz, 8H), 2.19 (t, J=2.8 Hz, 3H), 1.67-1.58 (m, 15H), 1.57-1.40 (m, 12H), 1.35-1.20 (m, 87H), 0.88 (t, J=7.0 Hz, 12H), ESI-MS: MW for C₆₅H₁₃₁N₃O₂ [M+H]⁺ calc. 987.03, found 986.87.

### Synthesis of N,N′-(((5-hydroxypentyl)azanediyl)bis(octane-8,1-diyl))bis(N-dodecyldodecanamide) (I-68)

N,N′-(((5-hydroxypentyl)azanediyl)bis(octane-8,1-diyl))bis(N-dodecyldodecanamide) (I-68) was prepared according to the general procedure from Int 4-2 (700 mg, 1.25 mmol) and 5-aminopentan-1-ol (44 mg, 0.425 mmol). The product was obtained as pale-yellow oil (471 mg, 0.45 mmol, 72%). ¹H NMR (600 MHz, CDCl₃) δ 3.63 (t, J=6.5 Hz, 2H), 3.27 (dd, J=10.7, 4.5 Hz, 4H), 3.22-3.15 (m, 4H), 2.43-2.33 (m, 6H), 2.29-2.23 (m, 4H), 1.69 (s, 4H), 1.65-1.34 (m, 24H), 1.34-1.19 (m, 88H), 0.91-0.84 (m, 12H), ESI-MS: MW for C₆₉H₁₃₉N₃O₃ [M+H]⁺ calc. 1059.09, found 1058.89.

### Example 60

### Synthetic Pathway for Compounds I-69 & I-71

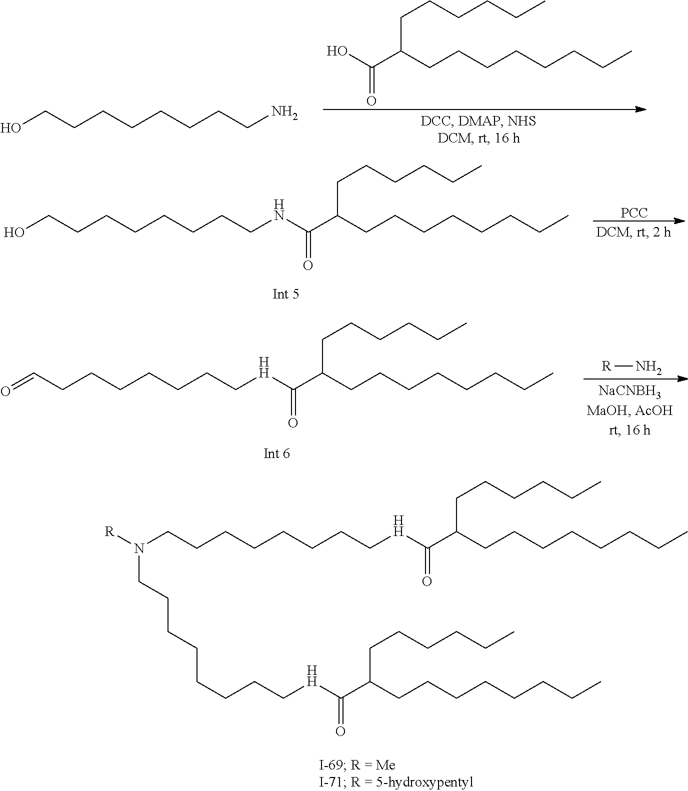

### Synthesis of N,N′-((methylazanediyl)bis(octane-8,1-diyl))bis(2-hexyldecanamide) (I-69)

N,N′-((methylazanediyl)bis(octane-8,1-diyl))bis(2-hexyldecanamide) (I-69) was prepared according to the general procedures of example 59 from Int 6 (0.408 g, 1.06 mmol) and methylamine (2M in THF, 0.181 mL, 0.36 mmol). The product was obtained as pale-yellow oil (210 mg, 0.27 mmol, 26%). ¹H NMR (400 MHz, CDCl₃) δ 3.32-3.23 (m, 4H), 3.23-3.14 (m, 4H), 2.75-2.37 (m, 7H), 2.26 (t, J=8.3 Hz, 4H), 1.71-1.58 (m, 8H), 1.50 (dq, J=14.4, 7.2, 6.8 Hz, 8H), 1.37-1.18 (m, 52H), 0.92-0.82 (m, 12H). ESI-MS: MW for C₄₉H₉₉N₃O₂ [M+H]⁺ calc. 762.78, found 762.88.

### Synthesis of N,N′-(((5-hydroxypentyl)azanediyl)bis(octane-8,1-diyl))bis(2-hexyldecanamide) (I-71)

N,N′-(((5-hydroxypentyl)azanediyl)bis(octane-8,1-diyl))bis(2-hexyldecanamide) (I-71) was prepared according to the general procedures of example 59 from Int 6 (458 mg, 1.20 mmol) and 5-Amino-1-pentanol (22 mg, 0.40 mmol). The product was obtained as pale-yellow oil (250 mg, 0.30 mmol, 25%). ¹H NMR (400 MHz, CDCl₃-d) δ 5.52 (t, J=5.6 Hz, 2H), 3.66 (t, J=6.2 Hz, 2H), 3.24 (q, J=6.6 Hz, 4H), 3.02-2.94 (m, 2H), 2.77 (m, 5H), 2.04-1.89 (m, 4H), 1.67-1.13 (m, 86H), 0.94-0.78 (m, 13H). ESI-MS: MW for C₅₃H₁₀₇N₃O₃ [M+H]⁺ calc. 834.84, found 834.98.

### Example 61

### Synthetic Pathway for Compounds I-70 & I-72

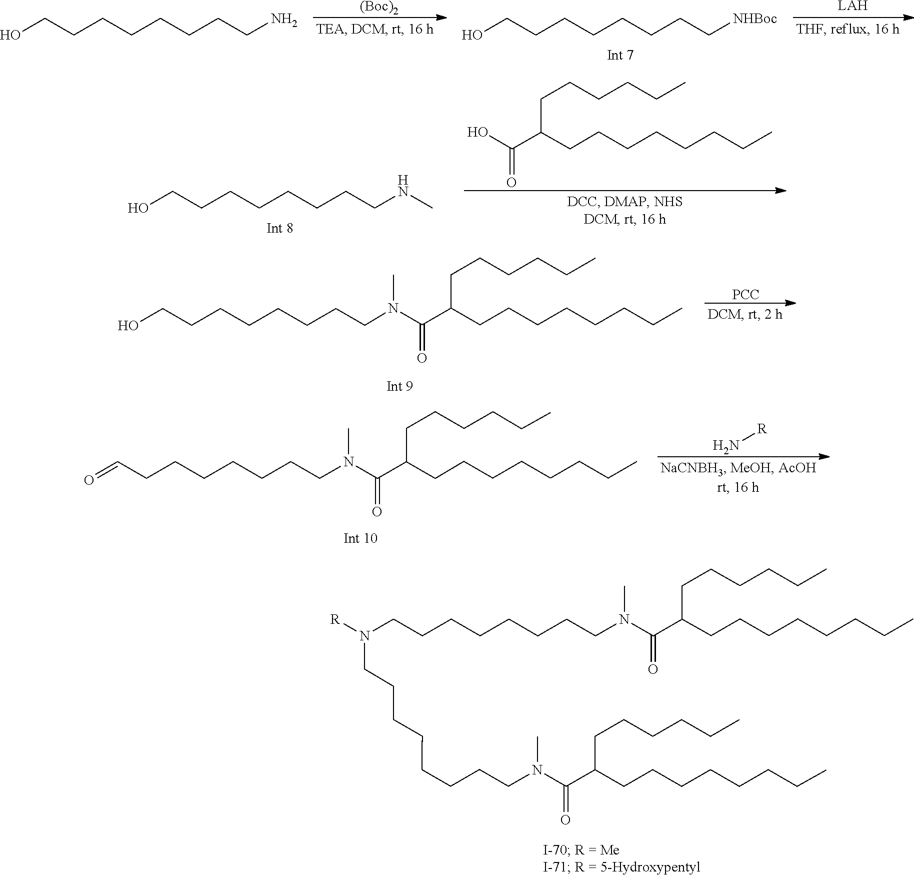

### Synthesis of tert-butyl (8-hydroxyoctyl)carbamate (Int 7)

To a solution of 8-amino-1-octanol (10 g, 68.9 mmol) in DCM (100 mL) was added triethylamine (13.9 g, 137.74 mmol) at RT under N₂. Then, Boc-anhydride (16.5 g, 75.75 mmol) was added dropwise at 0° C. over 10 min. The resulting mixture was stirred at RT for 16 h. Water (200 mL) was then added to the reaction mixture and stirred for 20 min. The organic layer was separated, and the aqueous layer was extracted with DCM (100 mL×2). The combined organic layer was dried over Na₂SO₄, filtered, and concentrated under reduced pressure to yield the crude product. The obtained crude was purified by flash column chromatography (Hex/EtOAc 100:0 to 55:45) to yield Int 7 as solid (15.2 g, 61.94 mmol, 90%). ESI-MS: MW for C₁₃H₂₇NO₃Na [M+Na]⁺ calc. 268.35, found 268.31.

### Synthesis of 8-(methylamino)octan-1-ol (Int 8)

To a stirred suspension of LAH (10.12 g, 266.80 mmol) in dry THF (100 mL) at 0° C. under N₂, was added a solution of Int 7 (15 g, 61.13 mmol) in dry THF (50 mL) dropwise over 30 min and the resulting suspension was refluxed for 16 h. The reaction mixture was then cooled to 0° C. and water (10 mL) was added dropwise. Subsequently, a solution of NaOH (15%, 10 mL) followed by water (10 mL) were added dropwise. To the resulting cake solution, MgSO4 was added, and the mixture stirred at rt for 30 min. The precipitate was discarded by filtration and the filtrate was washed with brine and dried over Na₂SO₄. The organic layer was evaporated under reduced pressure to afford the crude 8-(methylamino)octan-1-ol (Int 8) (7.2 g, 45.20 mmol, 74%). ESI-MS: MW for C₉H₂₁NO [M+H]⁺ calc. 160.28; found 160.21.

### Synthesis of 2-hexyl-N-(8-hydroxyoctyl)-N-methyldecanamide (Int 9)

Int 9 was prepared according to the general procedures of example 59 from Int 8 (2.6 g, 16.38 mmol) and 2-hexyldecanoic acid (3.78 g, 14.7 mmol). The product was obtained as colourless oil (2.2 g, 5.53 mmol, 34%). ESI-MS: MW for C₂₅H₅₁NO₂ [M+H]⁺ calc. 398.70; found 398.41.

### Synthesis of 2-hexyl-N-methyl-N-(8-oxooctyl)decanamide (Int 10)

According to the general procedures of example 59, Int 9 (2.05 g, 5.16 mmol) was converted into Int 10. The product was obtained as colourless oil (1.38 g, 3.49 mmol, 67.5%). ESI-MS: MW for C₂₅H₄₉NO₂ [M+H]⁺ calc. 396.68; found 396.45.

### Synthesis of N,N′-((methylazanediyl)bis(octane-8,1-diyl))bis(2-hexyl-N-methyldecanamide) (I-70)

N,N′-((methylazanediyl)bis(octane-8,1-diyl))bis(2-hexyl-N-methyldecanamide) (I-70) was prepared according to the general procedure of example 59 from Int 10 (500 mg, 1.26 mmol) and methylamine (2 M in MeOH, 13.4 mg, 0.225 mL, 0.43 mmol). The product was obtained as pale-yellow oil (200 mg, 0.25 mmol, 20%). ¹H NMR (400 MHz, CDCl₃) δ 3.40-3.34 (m, 2H), 3.31-3.24 (m, 2H), 3.01 (s, 3H), 2.92 (s, 2H), 2.68-2.22 (m, 8H), 1.78-1.46 (m, 16H), 1.45-1.36 (m, 4H), 1.27 (m, 53H), 0.87 (t, J=6.8 Hz, 12H). ESI-MS: MW for C₅₁H₁₀₃N₃O₂ [M+H]⁺ calc. 790.81; found 790.73.

### Synthesis of N,N′-(((5-hydroxypentyl)azanediyl)bis(octane-8,1-diyl))bis(2-hexyl-N-methyldecanamide) (I-72)

N,N′-(((5-hydroxypentyl)azanediyl)bis(octane-8,1-diyl))bis(2-hexyl-N-methyldecanamide) (I-72) was prepared according to the general procedure of example 59 from Int-10 (500 mg, 1.26 mmol) and 5-Amino-1-pentanol (44.4 mg, 0.43 mmol). The product was obtained as pale-yellow oil (235 mg, 0.27 mmol, 22%). ¹H NMR (400 MHz, CDCl₃) δ 3.64 (t, J=6.5 Hz, 2H), 3.41-3.34 (m, 2H), 3.32-3.25 (m, 2H), 3.01 (s, 3H), 2.92 (s, 3H), 2.64-2.51 (m, 2H), 2.49-2.34 (m, 6H), 1.74 (bs, 6H), 1.65-1.53 (m, 8H), 1.43 (m, 14H), 1.26 (m, 56H), 0.87 (t, J=6.8 Hz, 12H). ESI-MS: MW for C₅₅H₁₁₁N₃O₃ [M+H]⁺ calc. 862.87; found 862.69.

### Example 62

### Synthetic Pathway for Compounds I-75 to I-78

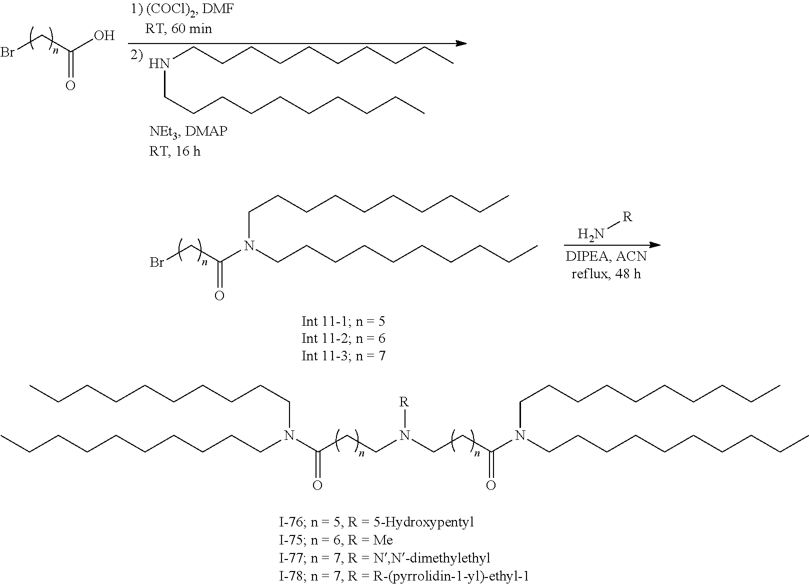

### General Procedure for the Preparation of Int 11-1, Int 11-2, & Int 11-3

To a solution of the appropriate carboxylic acid (1.0 eq.) in DCM (2 mL/mmol) were added a catalytical amount of DMF and oxalyl chloride (3.0 eq). The reaction mixture was stirred at RT under N₂ until complete conversion. Excess oxalyl chloride and DCM were then evaporated under vacuum. A solution of this resulting acyl chloride in dry DCM (1 mL/mmol) was slowly added to a solution of didecylamine (1.1 eq), NEt₃ (6.0 eq), and DMAP (cat.) in dry DCM (3 mL/mmol). Then, the mixture was stirred for 16 h at RT under N₂. After concentrating under reduced pressure, the residue was partitioned between H₂O (10 mL/mmol) and ethyl acetate (10 mL/mmol). The aqueous phase was extracted with ethyl acetate, and the combined organic phase was dried over Na₂SO₄, and concentrated under reduced pressure. The crude product was purified by column chromatography (hexane/ethyl acetate, 100:0 to 80:20).

### General Procedure for the Preparation of Compounds I-77, I-78, I-75 & I-76

To a solution of Int 11-1, Int 11-2 or Int 11-3 (1.5 eq) in ACN (5.5 mL/mmol) were added DIPEA (3.8 eq.) and the desired alkylation reagent (1.0 eq). The reaction was carried out in a sealed tube at 80° C. for 24 h. More bromide (Int 11-1, Int 11-2 or Int 11-3) (0.5 eq, in ACN) (2.0 mL/mmol) was added to the reaction mixture, and it was stirred at 80° C. for another 24 h. The reaction mixture was then cooled to RT and concentrated under reduced pressure. The crude product was purified by column chromatography (Hex/1% NEt₃ in EtOAc, 95:5 to 0:100).

### Synthesis of 6,6′-((5-hydroxypentyl)azanediyl)bis(N,N-didecylhexanamide) (I-77)

6,6′-((5-hydroxypentyl)azanediyl)bis(N,N-didecylhexanamide) (I-77) was prepared according to the general procedure from Int 11-1 (700 mg, 1.47 mmol) and 5-aminopentan-1-ol (0.053 g, 0.51 mmol). The product was obtained as pale-yellow oil (471 mg, (0.45 mmol, 72%). ¹H NMR (400 MHz, CDCl₃) δ 3.63 (t, J=6.5 Hz, 2H), 3.32-3.23 (m, 4H), 3.23-3.14 (m, 4H), 2.44-2.34 (m, 6H), 2.27 (t, J=7.5 Hz, 4H), 1.79-1.39 (m, 26H), 1.26 (m, 59H), 0.88 (td, J=6.8, 3.1 Hz, 12H). ESI-MS: MW for C₅₇H₁₅N₃O₃ [M+H]⁺ calc. 890.90, found 890.81.

### Synthesis of 7,7′-(methylazanediyl)bis(N,N-didecylheptanamide) (I-75)

7,7′-(methylazanediyl)bis(N,N-didecylheptanamide) (I-75) was prepared according to the general procedure from Int 11-2 (800 mg, 1.63 mmol) and methylamine (0.37 mL, 0.74 mmol, 2 M in MeOH). The product was obtained as pale-yellow oil (368 mg, 0.43 mmol, 43%). ¹H NMR (400 MHz, CDCl₃) δ 3.30-3.24 (m, 4H), 3.21-3.15 (m, 4H), 2.27 (dd, J=15.1, 7.5 Hz, 8H), 2.18 (s, 3H), 1.72-1.58 (m, I0H), 1.57-1.41 (m, 13H), 1.38-1.18 (m, 67H), 0.88 (td, J=6.8, 3.1 Hz, 12H), ESI-MS: MW for C₅₅H₁₁₁N₃O₂[M+H]⁺ calc. 846.88, found 846.51.

### Synthesis of 8,8′-((2-(dimethylamino)ethyl)azanediyl)bis(N,N-didecyloctanamide) (I-77)

8,8′-((2-(dimethylamino)ethyl)azanediyl)bis(N,N-didecyloctanamide) (I-77) was prepared according to the general procedure from Int 11-3 (800 mg, 1.59 mmol) and N,N-dimethylethyldiamine (0.07 mL, 0.7 mmol). The product was obtained as pale-yellow oil (197 mg, 0.21 mmol, 30%). ¹H NMR (400 MHz, CDCl₃) δ 3.31-3.23 (m, 4H), 3.23-3.13 (m, 4H), 2.63-2.51 (m, 2H), 2.49-2.35 (m, 6H), 2.31-2.20 (m, I0H), 1.84 (s, 3H), 1.67-1.58 (m, 4H), 1.57-1.39 (m, 12H), 1.28 (mi, 71H), 0.88 (td, J=6.8, 3.1 Hz, 12H), ESI-MS: MW for C₆₀H₁₂₂N₄O₂ [M+H]⁺ calc. 931.97, found 931.98.

### Synthesis of 8,8′-((2-(pyrrolidin-1-yl)ethyl)azanediyl)bis(N,N-didecyloctanamide) (I-78)

8,8′-((2-(pyrrolidin-1-yl)ethyl)azanediyl)bis(N,N-didecyloctanamide) (I-78) was prepared according to the general procedure from Int 11-3 (700 mg, 1.39 mmol) and 1-(2-aminoethyl)pyrrolidine (0.08 mL, 0.7 mmol). The product was obtained as pale-yellow oil (260 mg, 0.27 mmol, 39%). ¹H NMR (400 MHz, CDCl₃) δ 3.32-3.23 (m, 4H), 3.22-3.12 (m, 4H), 2.64-2.47 (m, 7H), 2.45-2.36 (m, 4H), 2.30-2.23 (m, 4H), 1.84-1.68 (m, 9H), 1.67-1.58 (m, 4H), 1.57-1.37 (m, 12H), 1.26 (mi, 71H), 0.88 (td, J=6.7, 3.0 Hz, 12H), ESI-MS: MW for C₆₂H₁₂₄N₄O₂ [M+H]⁺ calc. 957.98, found 957.92.

### Example 63

### Synthetic Pathway for Compounds I-73 & I-74

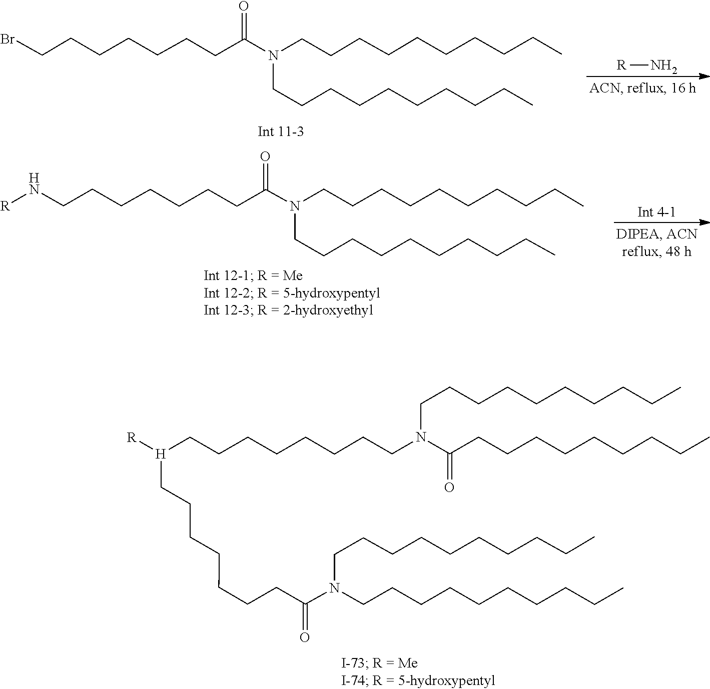

### General Procedure for the Preparation of Int 12-1, Int 12-2, & Int 12-3

A solution of Int 11-3 (1 eq) and the appropriate amine (5 eq) in acetonitrile (10 mL/mmol) was heated to reflux for overnight. The reaction mixture was concentrated, and the crude material was purified by column chromatography (Hex/EtOAc 95:5 to 0:100 then DCM/3% NH₃ in MeOH 100:0 to 90:10).

### General Procedure for the Preparation of I-73 & I-74

A mixture of Int 12-1 or Int 12-2 (1 eq), Int 4-1 (1.2 eq), and DIPEA (4 eq) in ACN (10 mL/mmol) was heated to reflux for overnight. The reaction mixture was then concentrated, and the crude material was purified via column chromatography (Hex/1% NEt₃ in EtOAc 100:0 to 35:65).

### Synthesis of N-decyl-N-(8-((8-(didecylamino)-8 oxooctyl) (methyl) amino) octyl)decanamide (I-73)

N-decyl-N-(8-((8-(didecylamino)-8 oxooctyl) (methyl) amino) octyl)decanamide (I-73) was prepared according to the general procedure from Int 12-1 (0.4 g, 0.88 mmol) and Int 4-1 (0.53 g, 1.06 mmol). The product was obtained as pale-yellow oil (0.38 g, 0.43 mmol 49.3%). ¹H NMR (400 MHz, CDCl₃) δ 3.34-3.23 (m, 4H), 3.22-3.11 (m, 4H), 2.34-2.22 (m, 8H), 2.18 (s, 3H), 1.67-1.58 (m, 4H), 1.50 (m, 12H), 1.37-1.18 (m, 68H), 0.87 (t, J=6.8 Hz, 12H). ESI-MS: MW for C₅₇H₁₁₅N₃O₂ [M+H]⁺ calc. 874.91, found 874.56.

### Synthesis of N-decyl-N-(8-((8-(didecylamino)-8-oxooctyl)(5-hydroxypentyl)amino)octyl)decanamide (I-74)

N-decyl-N-(8-((8-(didecylamino)-8-oxooctyl)(5-hydroxypentyl)amino)octyl)decanamide (I-74) was prepared according to the general procedure from Int 12-2 (0.4 g, 0.76 mmol) and Int 4-1 (0.46 g, 0.91 mmol). The product was obtained as pale-yellow oil (0.4 g, 0.42 mmol 55.5%). ¹H NMR (400 MHz, CDCl₃) δ 3.63 (t, J=6.5 Hz, 2H), 3.32-3.24 (m, 4H), 3.22-3.13 (m, 4H), 2.45-2.32 (m, 6H), 2.32-2.22 (m, 4H), 1.78-1.56 (m, 11H), 1.56-1.36 (m, 16H), 1.36-1.18 (m, 68H), 0.87 (t, J=6.8 Hz, 13H). ESI-MS: MW for C₆₁H₁₂₃N₃O₃ [M+H]⁺ calc. 946.97, found 946.73.

### Example 64

### Synthetic Pathway for Compounds I-73 & I-74

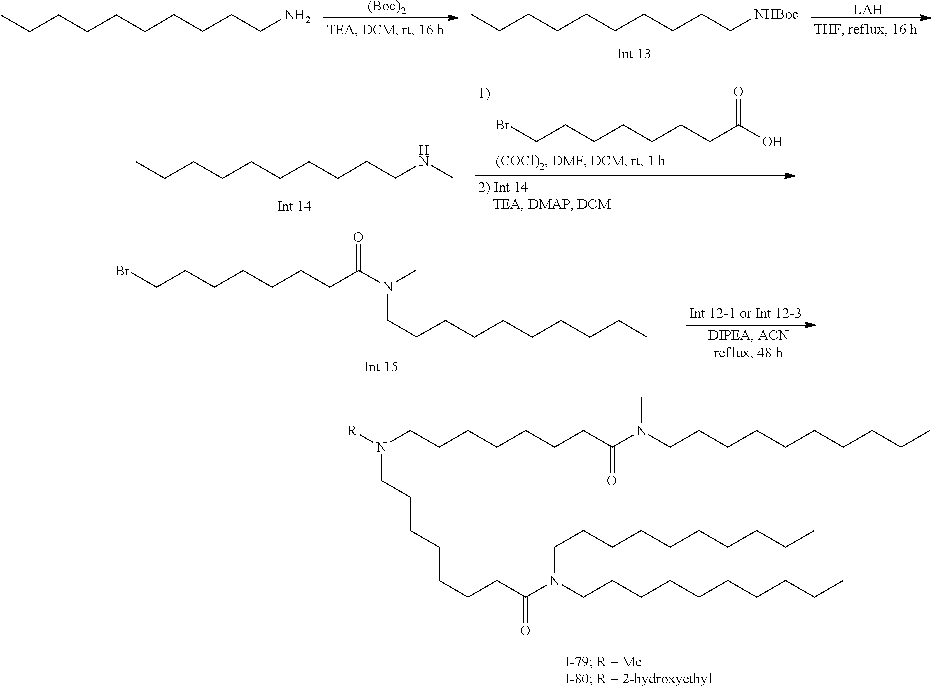

### Synthesis of tert-butyl decylcarbamate (Int 13)

To a solution of decan-1-amine (10 g, 63.57 mmol) in DCM (100 mL) was added triethylamine (12.9 g, 127.14 mmol), at RT under N₂. Then the Boc-anhydride (15.3 g, 69.93 mmol) was added dropwise at 0° C. over 10 min. The resulting mixture was stirred at RT for 16 h. Water (200 mL) was added to the reaction mixture and stirred for 20 min. The organic layer was separated, and the aqueous layer was extracted with DCM (100 mL×2). The combined organic layer was dried over Na₂SO₄, filtered, and concentrated under reduced pressure to yield the crude product. The obtained crude was purified by column chromatography (Hex/EtOAc 100:0 to 75:25). The product tert-butyl decylcarbamate Int 13 was obtained as solid (16 g, 62.15 mmol, 97%). ESI-MS: MW for C₁₅H₃₁NO₂ [M+Na]⁺ calc. 280.22; found 280.26.

### Synthesis of N-methyldecylamine (Int 14)

To a stirred suspension of LAH (5.9 g, 155.40 mmol) in dry THF (100 mL) at 0° C. under N₂, was added a solution of Int 13 (10 g, 38.85 mmol) in dry THF (50 mL) dropwise over 30 min. The resulted suspension was refluxed for 16 h, and then it was cooled to 0° C. and water (10 mL) was added dropwise. subsequently, a solution of NaOH (15%, 10 mL) followed by water (10 mL) were added dropwise. To the resulting cake solution, MgSO4 was added, and the resulting mixture stirred at rt for 30 min. The precipitate was discarded by filtration, and filtrate was washed with brine and dried over Na₂SO₄. The organic layer was evaporated under reduced pressure to afford the crude Int 14 (5.5 g 32.10 mmol, 82.6%). ESI-MS: MW for C_{II}H₂₅N [M+H]⁺ calc. 172.21; found 172.25.

### Synthesis of 8-bromo-N-decyl-N-methyloctanamide (Int 15)

Step 1: To a solution of 1-bromooctanoic acid (3.5 g, 15.69 mmol) in DCM (50 mL) was added DMF (3 drops) followed by oxalyl chloride (4 mL, 47.06 mmol). The reaction mixture was stirred at RT for 1 h. The reaction mixture was then concentrated to give 8-bromooctanoyl chloride which was used in the next step without further purification.

Step 2: To a solution of N-methyldecylamine (3 g, 17.25 mmol), triethylamine (13.2 mL, 94.12 mmol) and DMAP (cat.) in DCM (50 mL) was added a solution of 8-bromooctanoyl chloride (˜15.69 mmol) in DCM (20 mL). After stirring at RT overnight, the reaction mixture was concentrated and the remaining residue was partitioned between EtOAc and brine. The organic layer was separated, and the aqueous layer was extracted again with EtOAc. The combined organic layer was dried over Na₂SO₄, filtered, and concentrated. Purification via automated flash chromatography (5% to 35% EtOAc in hexanes) yielded Int 15 as pale-yellow oil (4.8 g, 12.75 mmol, 81%, 2 steps). ESI-MS: MW for C₁₉H₃₈BrNO [M+H]⁺ calc. 376.22; found 376.23.

### General Procedure for the Preparation of I-79 & I-80

A mixture of Int 12-1 or Int 12-3 (1 eq), Int 15 (1.2 eq), and DIPEA (4 eq) in ACN (10 mL/mmol) was heated to reflux for overnight. The reaction mixture was then concentrated under reduced pressure, and the crude material was purified via automated flash chromatography (Hex/1% NEt₃ in EtOAc 95:5 to 35:65).

### Synthesis of N,N-didecyl-8-((8-(decyl(methyl)amino)-8-oxooctyl)(methyl)amino)octanamide (I-79)

N,N-didecyl-8-((8-(decyl(methyl)amino)-8-oxooctyl)(methyl)amino)octanamide (I-79) was prepared according the general procedure from Int 12-1 (0.5 g, 1.10 mmol), and Int 15 (0.5 g, 1.33 mmol). The product was obtained as pale-yellow oil (0.25 g, 0.33 mmol 30.3%). ¹H NMR (400 MHz, CDCl₃) δ 3.38-3.14 (m, 6H), 2.92 (d, J=23.1 Hz, 3H), 2.33-2.22 (m, 8H), 2.18 (s, 3H), 1.67-1.59 (m, 4H), 1.57-1.40 (m, 10H), 1.37-1.21 (m, 55H), 0.87 (q, J=3.8 Hz, 9H). ESI-MS: MW for C₄₈H₉₇N₃O₂ [M+H]⁺ calc. 748.77; found 748.68.

### Synthesis of N,N-didecyl-8-((8-(decyl(methyl)amino)-8-oxooctyl)(2-hydroxyethyl)amino)octanamide (I-80)

N,N-didecyl-8-((8-(decyl(methyl)amino)-8-oxooctyl)(2-hydroxyethyl)amino)octanamide (I-80) was prepared according to the general procedure from Int12-3 (0.5 g, 1.03 mmol) and Int-15 (0.47 g, 1.24 mmol). The product was obtained as pale-yellow oil (0.260 g, 0.33 mmol 32.4%). ¹H NMR (400 MHz, CDCl₃) δ 3.51 (t, J=5.4 Hz, 2H), 3.37-3.15 (m, 7H), 2.93 (d, J=23.4 Hz, 3H), 2.56 (t, J=5.3 Hz, 2H), 2.47-2.37 (m, 4H), 2.33-2.22 (m, 4H), 1.62 (s, 5H), 1.57-1.38 (m, 11H), 1.36-1.17 (m, 58H), 0.92-0.83 (m, 9H). ESI-MS: MW for C₄₉H₉₉N₃O₃ [M+H]⁺ calc. 778.78; found 778.85.

### Example 65

### 8,8′-((5-Hydroxypentyl)Azanediyl)Bis(N,N-Dinonyloctanamide) (Compound I-81)

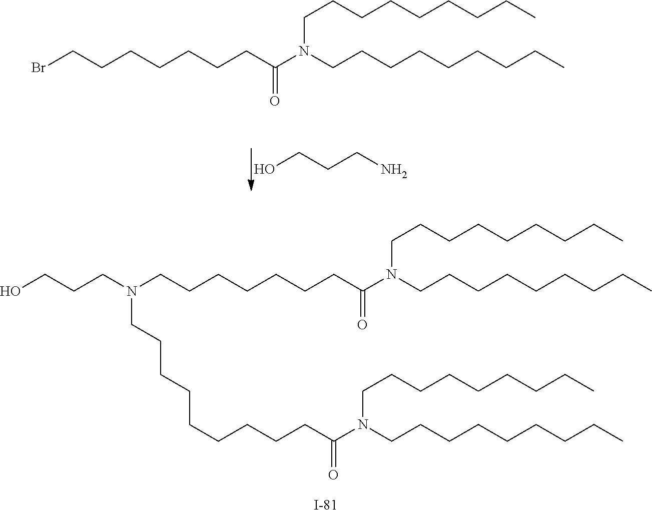

### Synthesis of 8,8′-((5-hydroxypentyl)azanediyl)bis(N,N-dinonyloctanamide) (I-81)

A mixture of 8-bromo-N,N-dinonyloctanamide (was prepared according to the general procedures of example 5, 0.7 mmol, 330 mg), 5-aminopentan-1-ol (0.42 mmol, 43 mg), DIEA (1.3 mmol, 0.23 mL), and potassium iodide (0.7 mmol, 116 mg) in ACN (4 mL) was heated at 75° C. for 48 h. The reaction mixture was concentrated and the crude material was purified via automated flash chromatography (5% to 65% EtOAc in hexanes) to give compound I-81 (88 mg, 28%). ¹H NMR (400 MHz, CDCl₃) δ 3.63 (t, J=6.5 Hz, 2H), 3.32-3.24 (m, 4H), 3.23-3.15 (m, 4H), 2.41-2.32 (m, 6H), 2.31-2.22 (m, 4H), 1.69-1.34 (m, 24H), 1.35-1.19 (m, 61H), 0.92-0.83 (m, 12H). ESI-MS: MW for C₅₇H₁₅N₃O₃ [M+H]⁺ Calc. 890.9; Found 891.0.

### Example 66

### 8,8′-((5-Hydroxypentyl)Azanediyl)Bis(N,N-Didecyl-2-Fluorooctanamide) (Compound I-82)

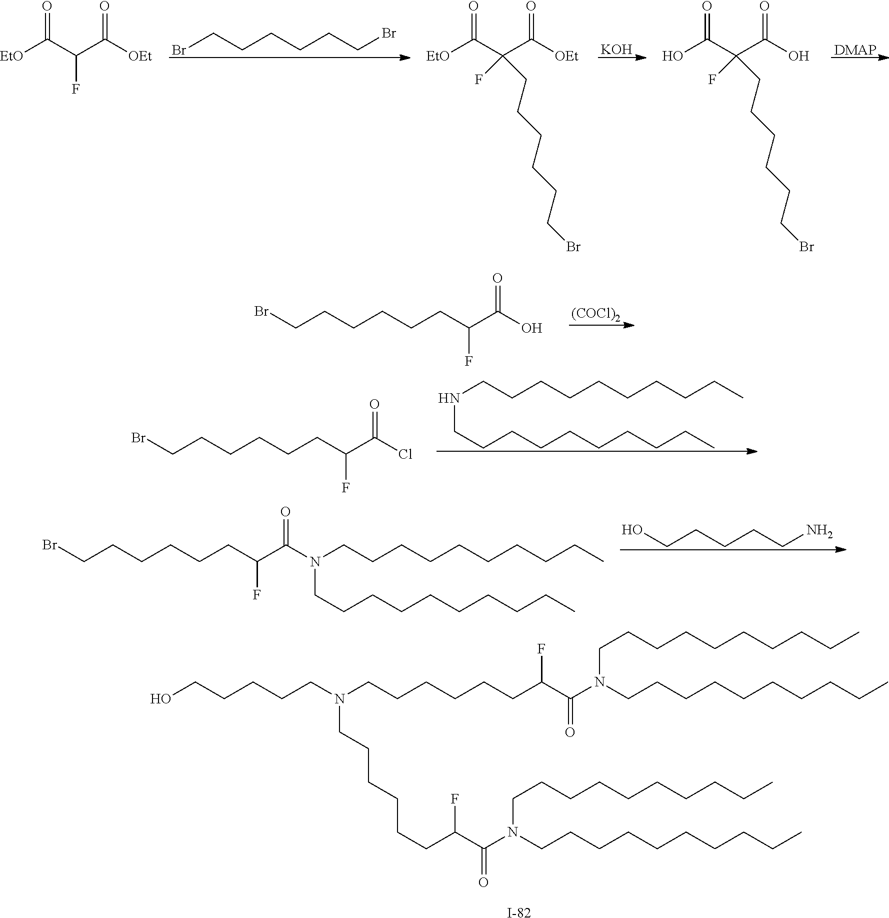

### Synthesis of diethyl 2-(6-bromohexyl)-2-fluoromalonate

A mixture of diethyl 2-fluoromalonate (56.1 mmol, 10.0 g), 1,6-dibromohexane (168 mmol, 26 mL), and sodium methoxide (61.7 mmol, 3.3 g) in EtOH (110 m,L) was stirred at room temperature for 24 h. The reaction mixture was concentrated and the crude material was partitioned between DCM and water. The organic layer was separated, dried over Na₂SO₄, filtered and concentrated. Purification via automated flash chromatography (0% to 25% EtOAc in hexanes) gave diethyl 2-(6-bromohexyl)-2-fluoromalonate (11.7 g, 61%).

### Synthesis of 2-(6-bromohexyl)-2-fluoromalonic acid

A mixture of diethyl 2-(6-bromohexyl)-2-fluoromalonate (5.9 mmol, 2.0 g) and KOH (11.8 mmol, 660 mg) in MeOH (20 mL), THF (2 mL), and water (4 mL) was stirred at room temperature for 3 h. The reaction mixture was concentrated and the crude material was diluted with 0.1M NaOH (20 mL). The aqueous layer was washed with DCM (3×10 mL), acidified with 1M HCl, then extracted with EtOAc (2×20 mL). The combined EtOAc layers were dried over Na₂SO₄, filtered and concentrated to give 2-(6-bromohexyl)-2-fluoromalonic acid (1.57 g, 94%) which was used in the next step without further purification.

### Synthesis of 8-bromo-2-fluorooctanoic acid

A mixture of 2-(6-bromohexyl)-2-fluoromalonic acid (4.1 mmol, 1.2 g) and DMAP (cat.) in DMF (3 mL), was heated at 180° C. for 12 min. The reaction mixture was partitioned between EtOAc and 1M HCl. The organic layer was separated, dried over Na₂SO₄, filtered and concentrated to give 8-bromo-2-fluorooctanoic acid (960 mg, 98%) which was used in the next step without further purification.

### Synthesis of 8-bromo-2-fluorooctanoyl chloride

A mixture of 8-bromo-2-fluorooctanoic acid (4.0 mmol, 960 mg), oxalyl chloride (12 mmol, 1.0 mL), and DMF (cat.) in DCM (10 mL) was stirred at room temperature for 20 min. The reaction mixture was concentrated to give 8-bromo-2-fluorooctanoyl chloride which was used in the next step without further purification.

### Synthesis of 8-bromo-N,N-didecyl-2-fluorooctanamide

To mixture of didecylamine (4.0 mmol, 1.2 g), triethylamine (24 mmol, 3.4 mL), and DMAP (cat.) in DCM (10 mL) was added a solution of crude 8-bromo-2-fluorooctanoyl chloride (4.0 mmol) in DCM (5 mL). The reaction mixture was stirred at room temperature for 1 h. The reaction was concentrated and purified via automated flash chromatography (5% to 25% EtOAc in hexanes) gave 8-bromo-N,N-didecyl-2-fluorooctanamide (1.2 g, 58% over 2 steps).

### Synthesis of 8,8′-((5-hydroxypentyl)azanediyl)bis(N,N-didecyl-2-fluorooctanamide) (I-82)

A mixture of 8-bromo-N,N-didecyl-2-fluorooctanamide (0.56 mmol, 290 mg), 5-aminopentanol (0.34 mmol, 35 mg), and DIEA (1.0 mmol, 0.18 mL), potassium iodide (0.56 mmol, 93 mg) in ACN (4 mL) was heated at 75° C. for 19 h. The reaction mixture was concentrated and the crude material was purified via automated flash chromatography (5% to 100% EtOAc in hexanes) to give compound I-82 (158 mg, 57%). ¹H NMR (400 MHz, CDCl₃) δ 5.03 (ddd, J=49.4, 8.5, 4.3 Hz, 2H), 3.64 (t, J=6.5 Hz, 2H), 3.37-3.25 (m, 6H), 3.25-3.12 (m, 2H), 2.43-2.33 (m, 6H), 1.97-1.68 (m, 6H), 1.64-1.34 (m, 26H), 1.33-1.22 (m, 62H), 0.93-0.83 (m, 12H). ESI-MS: MW for C₆₁H₁₂₁F₂N₃O₃[M+H]⁺ Calc. 982.9; Found 983.0.

### Example 67

### 8,8′-(Methylazanediyl)Bis(N,N-Didecyl-2-Fluorooctanamide) (Compound I-83)

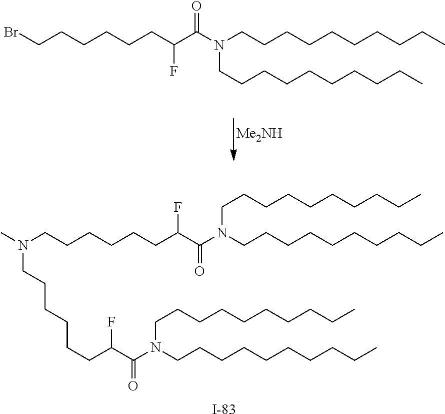

### Synthesis of 8,8′-(methylazanediyl)bis(N,N-didecyl-2-fluorooctanamide) (I-83)

A mixture of 8-bromo-N,N-didecyl-2-fluorooctanamide (was prepared according to the general procedures of example 66, 0.58 mmol, 300 mg), 8M methylamine in EtOH (0.36 mmol, 0.045 mL), and DIEA (1.1 mmol, 0.19 mL) in ACN (4 mL) was heated at 75° C. for 48 h. The reaction mixture was concentrated and the crude material was purified via automated flash chromatography (5% to 65% EtOAc in hexanes) to give compound I-83 (160 mg, 61%). ¹H NMR (400 MHz, CDCl₃) δ 5.14-4.93 (m, 2H), 3.40-3.24 (m, 6H), 3.24-3.10 (m, 2H), 2.33-2.25 (m, 4H), 2.19 (s, 3H), 1.99-1.71 (m, 4H), 1.62-1.17 (m, 80H), 0.92-0.84 (m, 12H). ESI-MS: MW for C₅₇H₁₁₃F₂N₃O₂[M+H]⁺ Calc. 910.9; Found 911.0.

### Example 68

### 2,2′-((5-Hydroxypentyl)Azanediyl)Bis(N,N-Didecylacetamide) (Compound I-84)

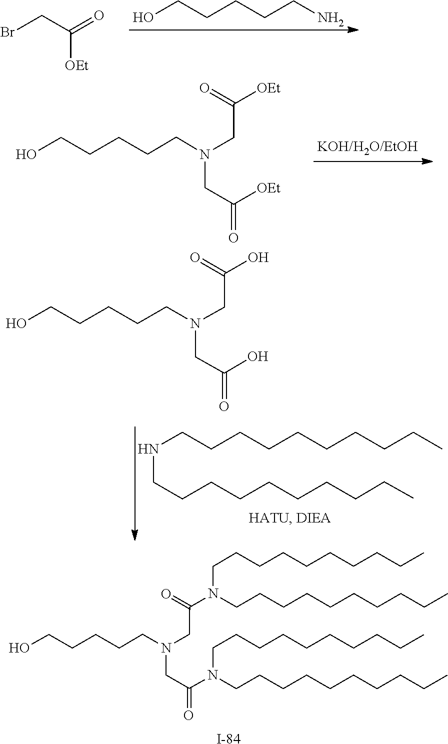

### Synthesis of diethyl 2,2′-((5-hydroxypentyl)azanediyl)diacetate

A mixture of ethyl 2-bromoacetate (14.2 mmol, 2.38 g, 1.57 ml), 5-aminopentan-1-ol (7.3 mmol, 0.75 g) DIEA (26.0 mmol, 2.5 mL), in ACN (10 mL) was stirred at 90° C. for 1.5 h in a pressure flask. The reaction mixture was concentrated, residue partitioned between water and EtOAc. The crude product, obtained after removal of EtOAc under vacuum, was purified on SiO₂ column (gradient; from Hexanes: Et₃N=99:1 to EtOAc: Et₃N=99:1) to give 1.6 g of pure diethyl 2,2′-((5-hydroxypentyl)azanediyl)diacetate (yield: 82%).

### Synthesis of 2,2′-((5-hydroxypentyl)azanediyl)diacetic acid

To diethyl 2,2′-((5-hydroxypentyl)azanediyl)diacetate (1.6 g, 5.8 mmol), dissolved in 10 ml of EtOH, 15 ml of 1M water solution of KOH were added. The reaction mixture was stirred for 1 h at room temperature. After that, EtOH was removed under vacuum and pH of the residue adjusted to 4. Water was removed under vacuum to give crude product that was used in the next step without any further purification.

### Synthesis of 2,2′-((5-hydroxypentyl)azanediyl)bis(N,N-didecylacetamide) (I-84)

To crude 2,2′-((5-hydroxypentyl)azanediyl)diacetic acid, Int-PB2 (0.26 mmol), THF (20 ml), DMF (2 ml), didecylamine (0.42 g, 1.4 mmol), DIEA (0.25 ml, 2.6 mmol) and HATU (0.34 g, 0.68 mmol) were added in that order. Reaction mixture was stirred at room temperature for 30 min. The reaction mixture was concentrated, and the crude material was purified via automated flash chromatography (5% to 75% EtOAc in hexanes with 1% Et₃N) to give I-84 (85 mg, 42%). ¹H NMR (400 MHz, CDCl₃) δ 3.62 (t, J=6.3 Hz, 2H), 3.50 (bs, 4H), 3.31-3.18 (m, 8H), 2.83-2.64 (m, 2H), 1.74-1.37 (m, 18H), 1.36-1.14 (m, 62H), 0.95-0.80 (m, 12H). ESI-MS: MW for C₄₉H₉₉N₃O₃ [M+H]⁺ Calc. 778.8; Found 778.8.

### Example 69

### 4,4′-((5-Hydroxypentyl)Azanediyl)Bis(N,N-Didecylbutanamide) (Compound I-85)

Compound I-85 was prepared according to the general procedures of example 68 to yield the desired product (445 mg, 76%). ¹H NMR (400 MHz, CDCl₃) δ 3.67-3.60 (m, 2H), 3.33-3.24 (m, 4H), 3.24-3.14 (m, 4H), 2.52-2.25 (m, 10H), 1.86-1.69 (m, 4H), 1.69-1.35 (m, 10H), 1.35-1.18 (m, 60H), 0.93-0.83 (m, 12H). ESI-MS: MW for C₅₃H₁₀₇N₃O₃ [M+H]⁺ Calc. 834.8; Found 834.9.

1. A compound having the following structure (I): 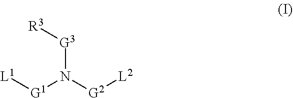
or a pharmaceutically acceptable salt, prodrug or stereoisomer thereof, wherein:
L¹ is —NR^{a}C(═O)R¹ or —C(═O)NR^{b}R^{c};
L² is —NR^{d}C(═O)R² or —C(═O)NR^{e}R^{f};
G¹ and G² are each independently C₂-C₁₂ alkylene;
G³ is C₁-C₂₄ alkylene;
R^{a}, R^{b}, R^{d} and R^{e} are each independently H or C₁-C₁₆ alkyl;
R^{c} and R^{f} are each independently C₁-C₁₆ alkyl;
R¹ and R² are each independently C₆-C₂₄ alkyl or C₆-C₂₄ alkenyl;
R³ is H, —OH, —N; —N(R⁴)R⁵; —C(═O)N(R⁴)R⁵; —N(R⁴)C(═O)R⁵; —N(R⁴)C(═O)OR⁵; and
R⁴ and R⁵ are each independently is H, C₁-C₁₂ alkyl, or R⁴ and R⁵, together with the nitrogen or carbon atom to which they are bound, form a 5 to 7-membered heterocyclic ring.

2-3. (canceled)

4. The compound of claim 1, wherein G³ is unsubstituted.

5-7. (canceled)

8. The compound of claim 1, having the following structure (IA): 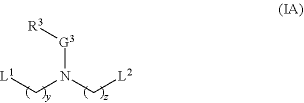
wherein y and z are each independently integers ranging from 2 to 12.

9. The compound of claim 1, wherein:
L¹ is —C(═O)NR^{b}R^{c}, and L² is —C(═O)NR^{e}R^{f} or
L¹ is —NR^{a}C(═O)R¹, and L² is —NR^{d}C(═O)R².

10-18. (canceled)

19. The compound of claim 1, wherein R¹ or R², or both, independently has one of the following structures:

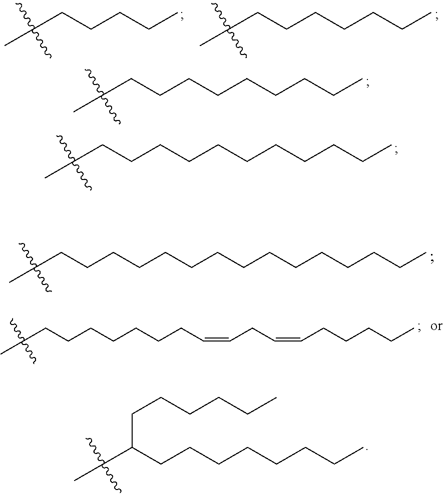

20-27. (canceled)

28. The compound of claim 1, wherein R³ is H.

29. The compound of claim 1, wherein R³ is —OH.

30-31. (canceled)

32. The compound of claim 1, wherein R³ is —N(R⁴)R⁵.

33-42. (canceled)

43. A compound having one of the following structures:

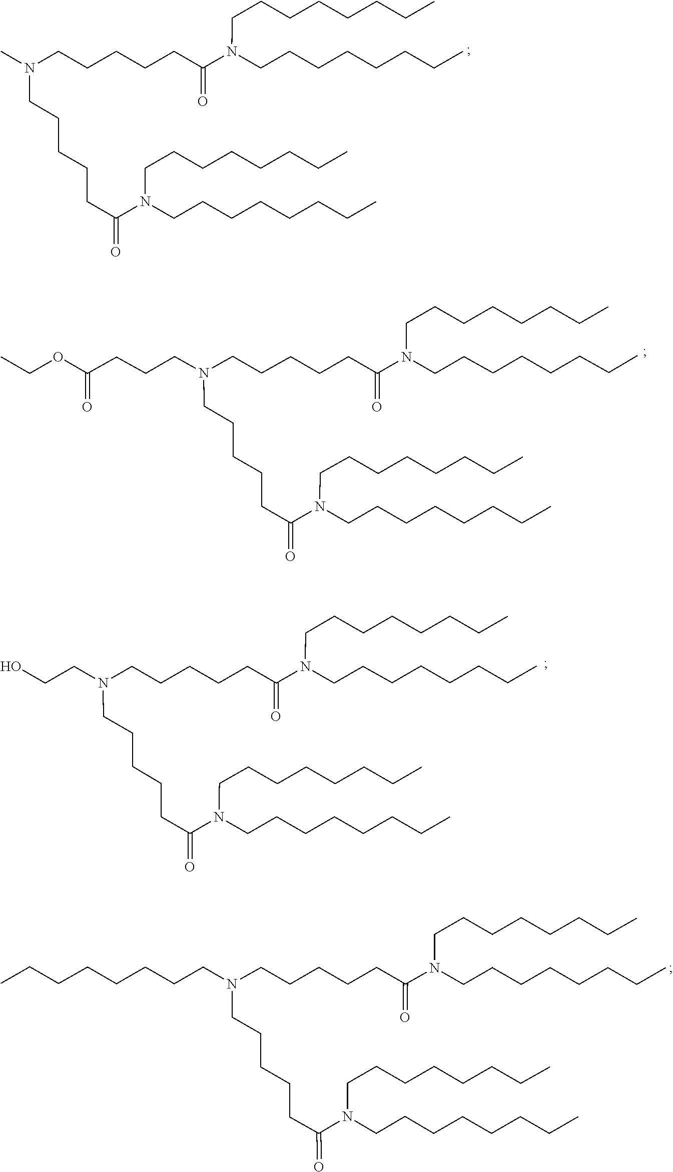

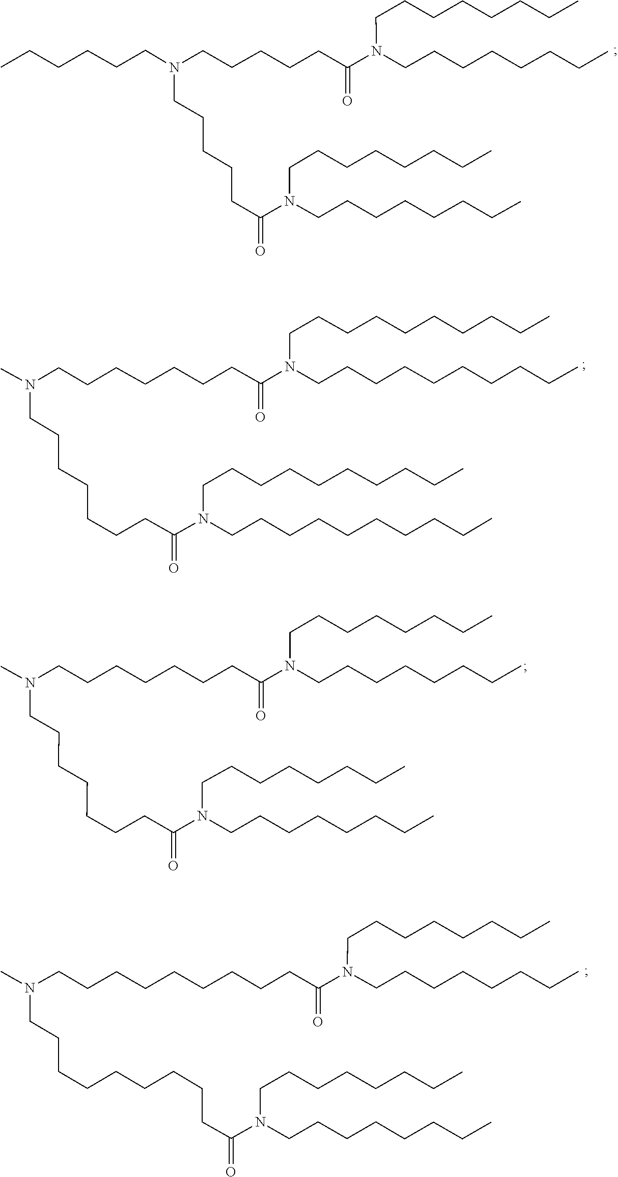

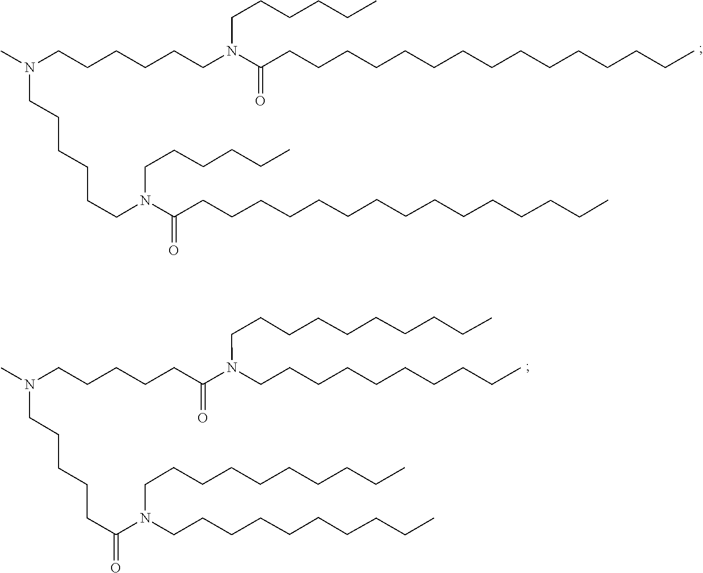

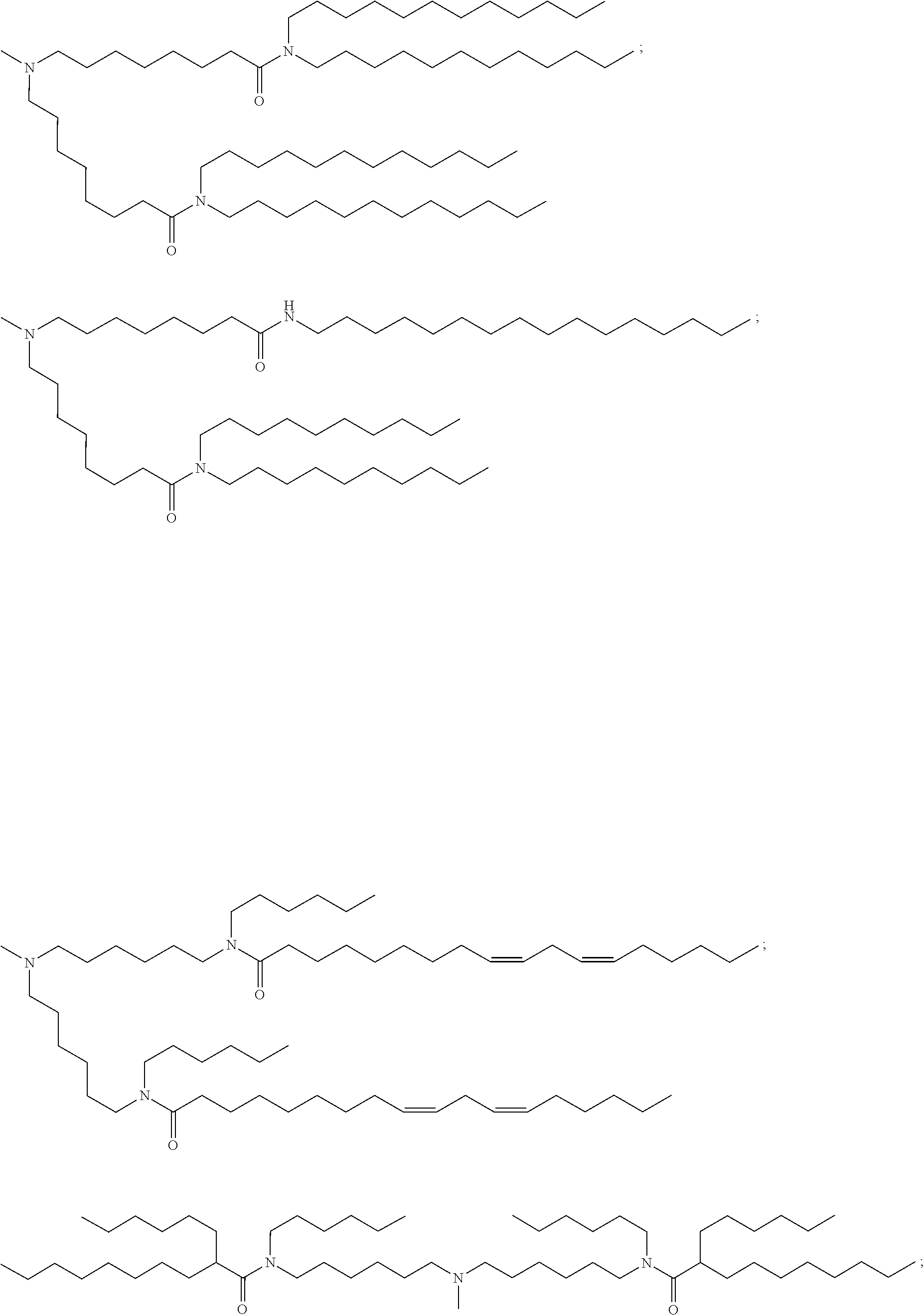

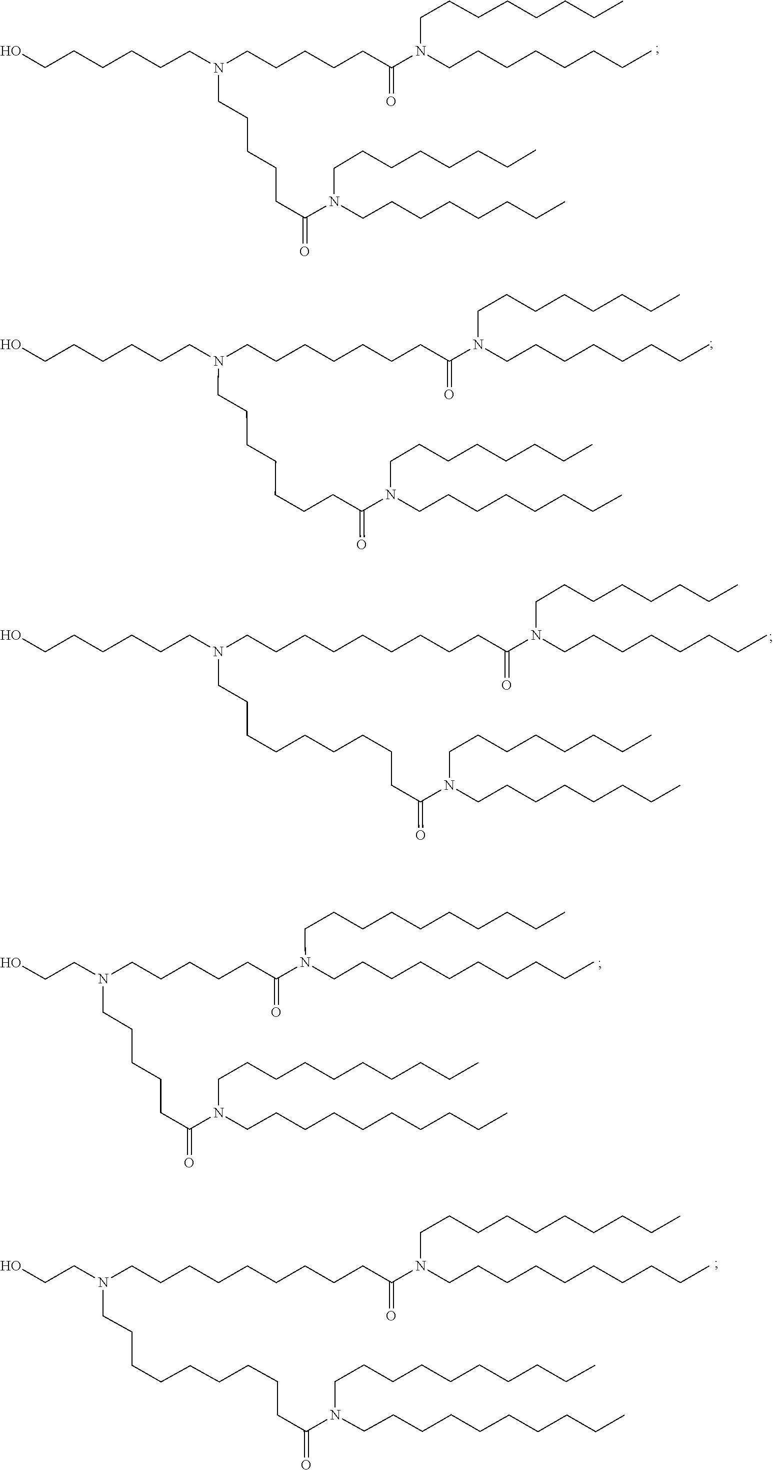 [SMILES: CCCCCCCCN(CCCCCCCC)C(=O)CCCCCN(CCCCCCO)CCCCCC(=O)N(CCCCCCCC)CCCCCCCC ; CCCCCCCCN(CCCCCCCC)C(=O)CCCCCCCN(CCCCCCO)CCCCCCCC(=O)N(CCCCCCCC)CCCCCCCC ; CCCCCCCCN(CCCCCCCC)C(=O)CCCCCCCCCN(CCCCCCO)CCCCCCCCCC(=O)N(CCCCCCCC)CCCCCCCC ; CCCCCCCCCCN(CCCCCCCCCC)C(=O)CCCCCN(CCO)CCCCCC(=O)N(CCCCCCCCCC)CCCCCCCCCC ; CCCCCCCCCCN(CCCCCCCCCC)C(=O)CCCCCCCCCN(CCO)CCCCCCCCCC(=O)N(CCCCCCCCCC)CCCCCCCCCC]

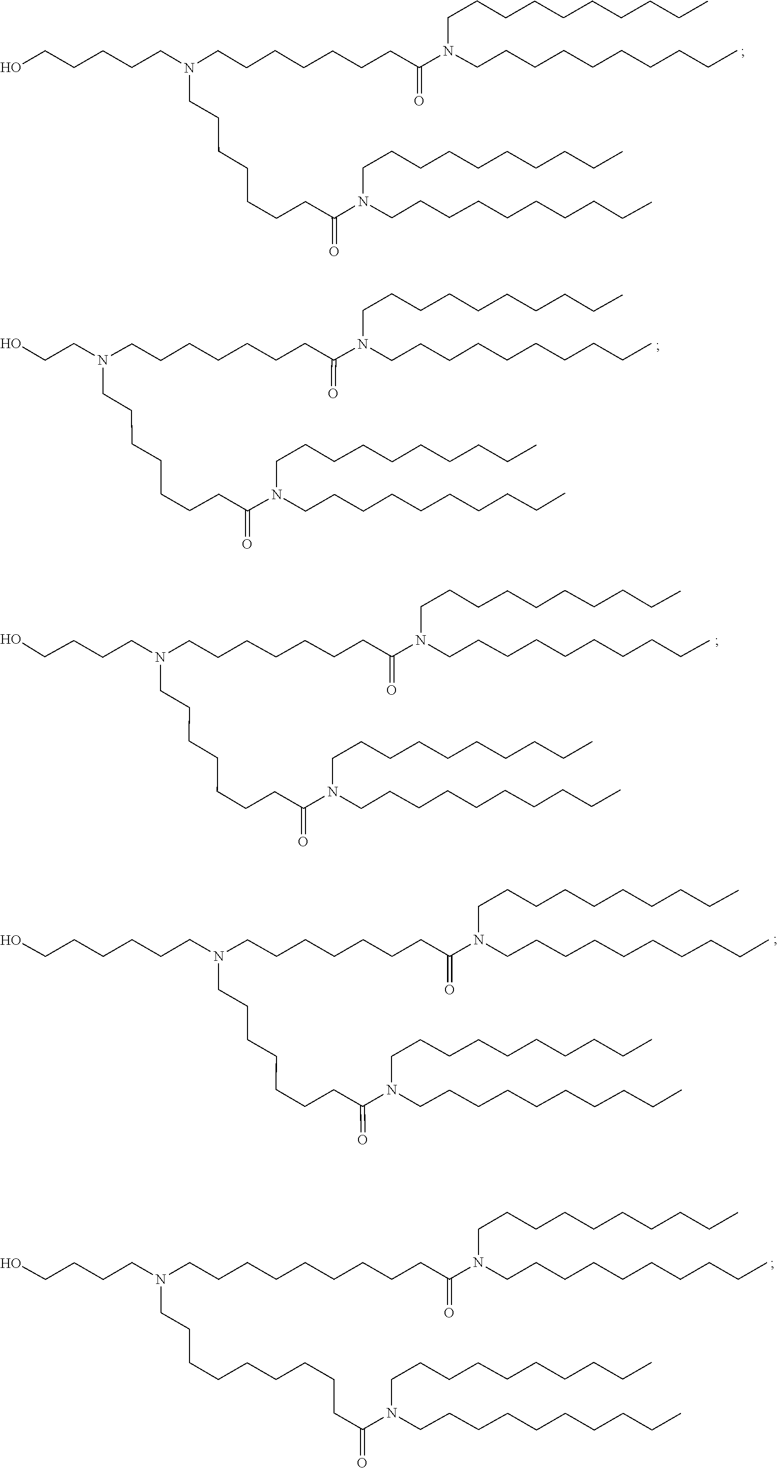 [SMILES: CCCCCCCCCCN(CCCCCCCCCC)C(=O)CCCCCCCN(CCCCCO)CCCCCCCC(=O)N(CCCCCCCCCC)CCCCCCCCCC ; CCCCCCCCCCN(CCCCCCCCCC)C(=O)CCCCCCCN(CCO)CCCCCCCC(=O)N(CCCCCCCCCC)CCCCCCCCCC ; CCCCCCCCCCN(CCCCCCCCCC)C(=O)CCCCCCCN(CCCCO)CCCCCCCC(=O)N(CCCCCCCCCC)CCCCCCCCCC ; CCCCCCCCCCN(CCCCCCCCCC)C(=O)CCCCCCCN(CCCCCCO)CCCCCCCC(=O)N(CCCCCCCCCC)CCCCCCCCCC ; CCCCCCCCCCN(CCCCCCCCCC)C(=O)CCCCCCCCCN(CCCCO)CCCCCCCCCC(=O)N(CCCCCCCCCC)CCCCCCCCCC]

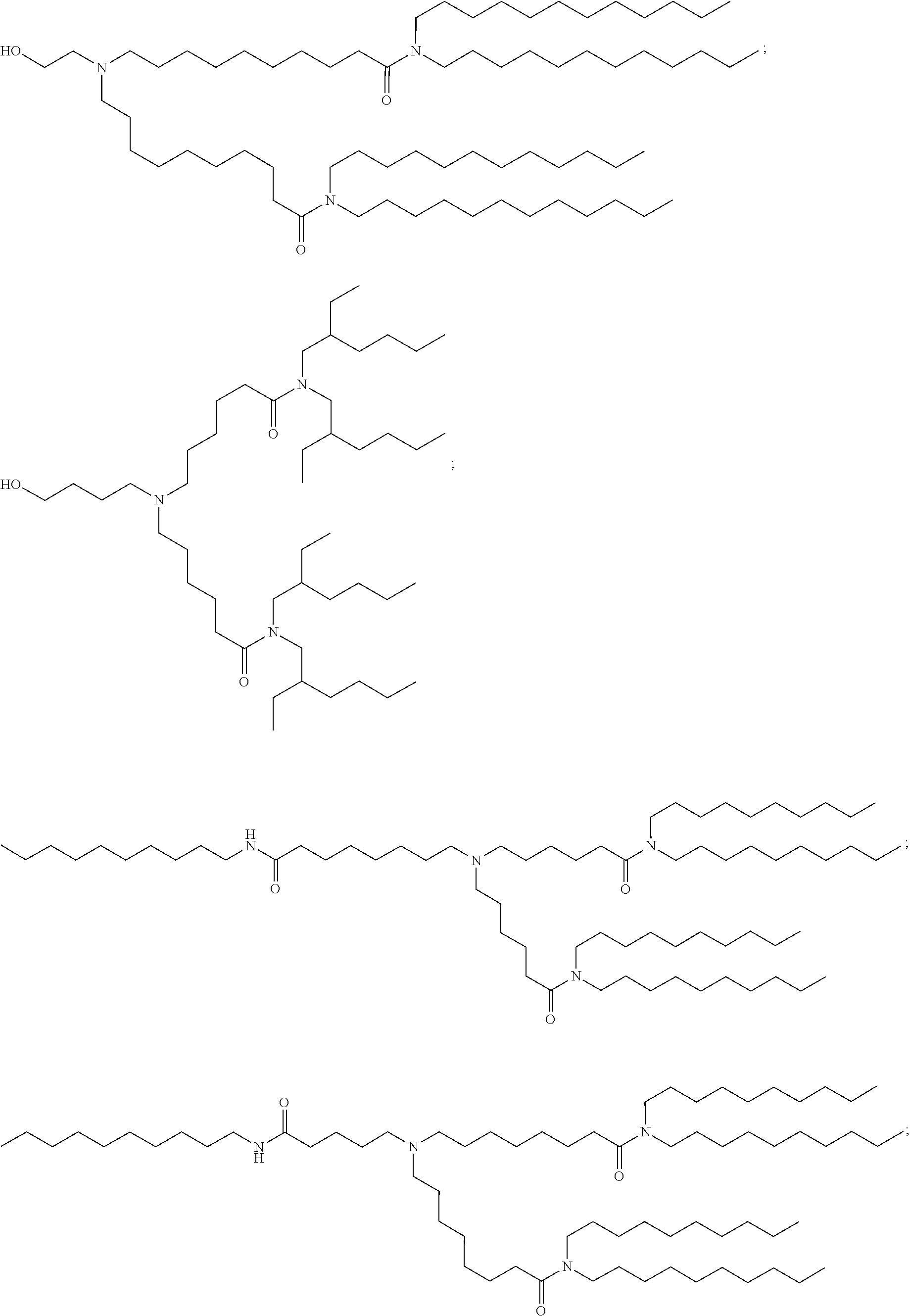

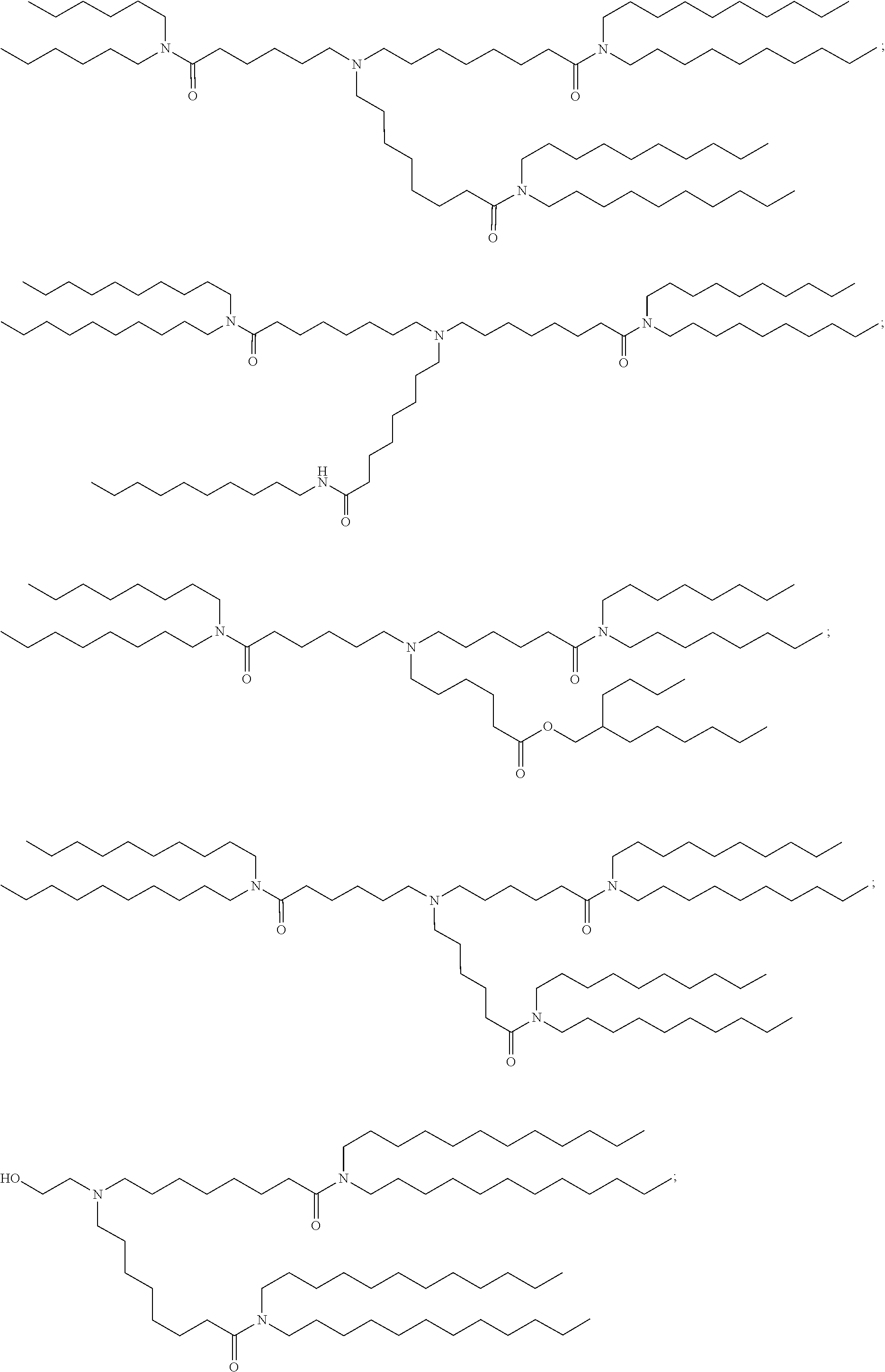

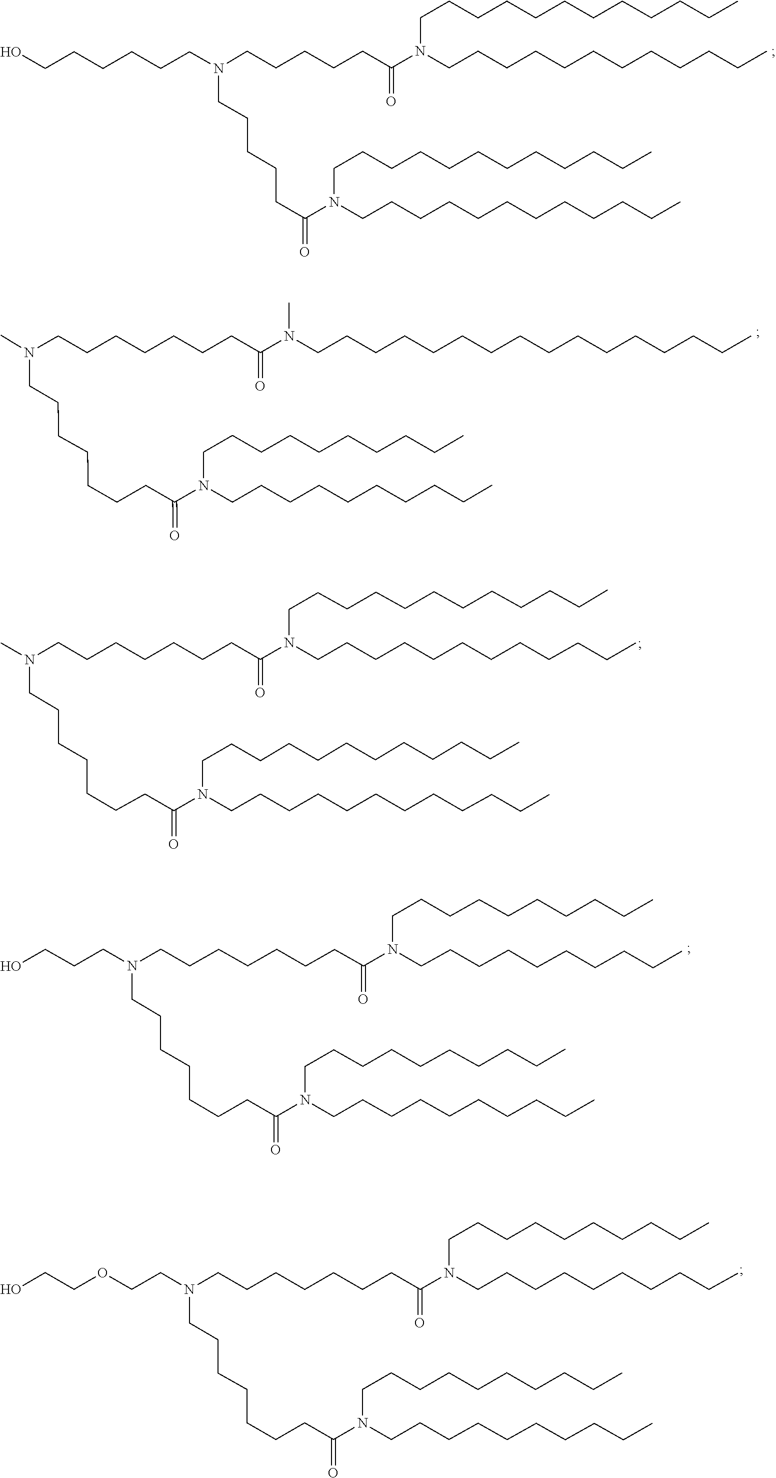

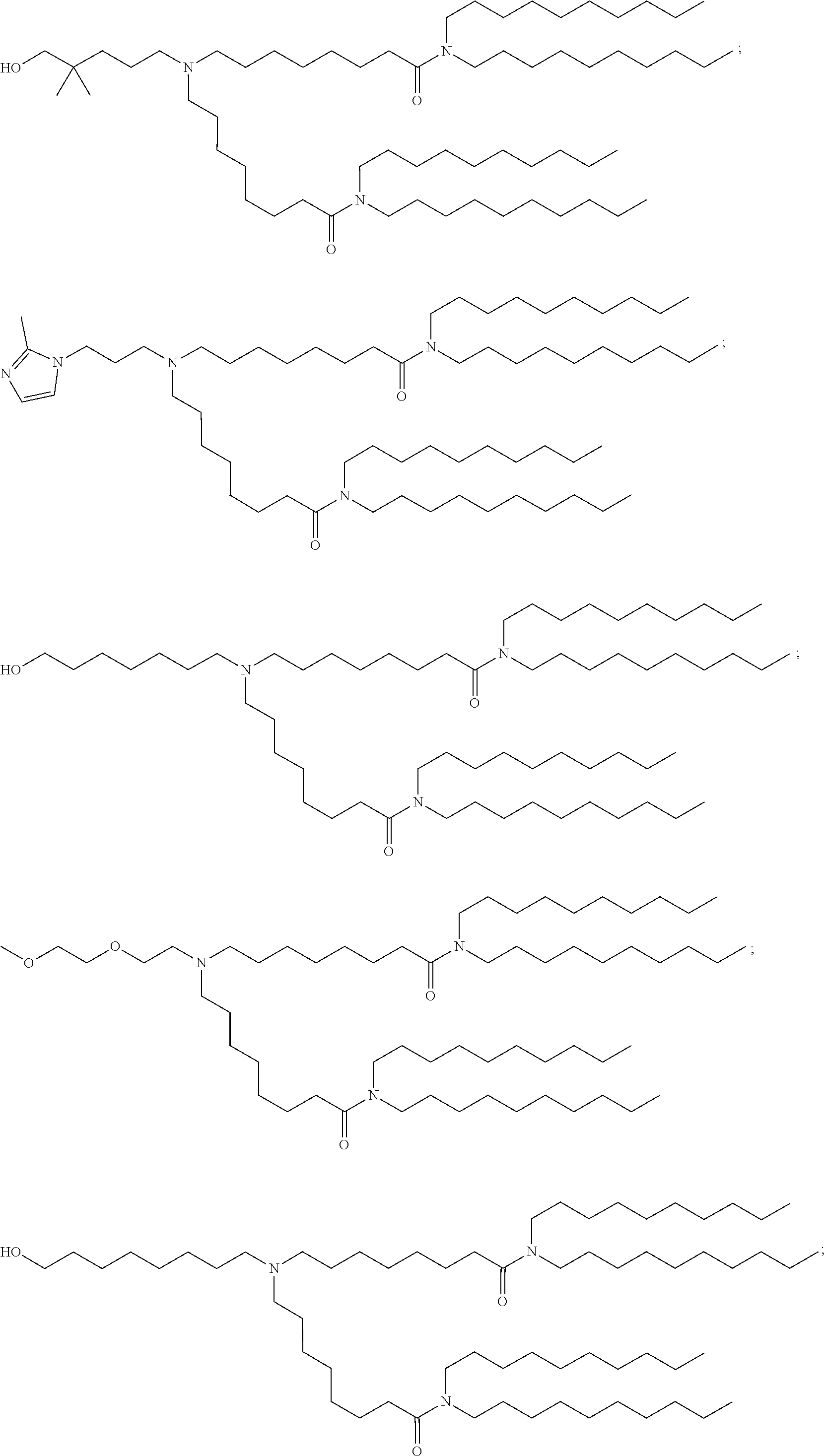

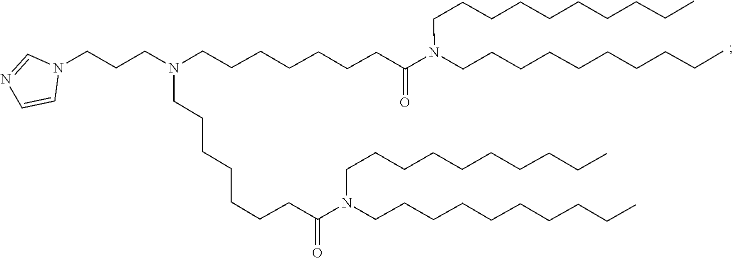 [SMILES: CCCCCCCCCCN(CCCCCCCCCC)C(=O)CCCCCCCN(CCCCCCCC(=O)N(CCCCCCCCCC)CCCCCCCCCC)CCCn1ccnc1]

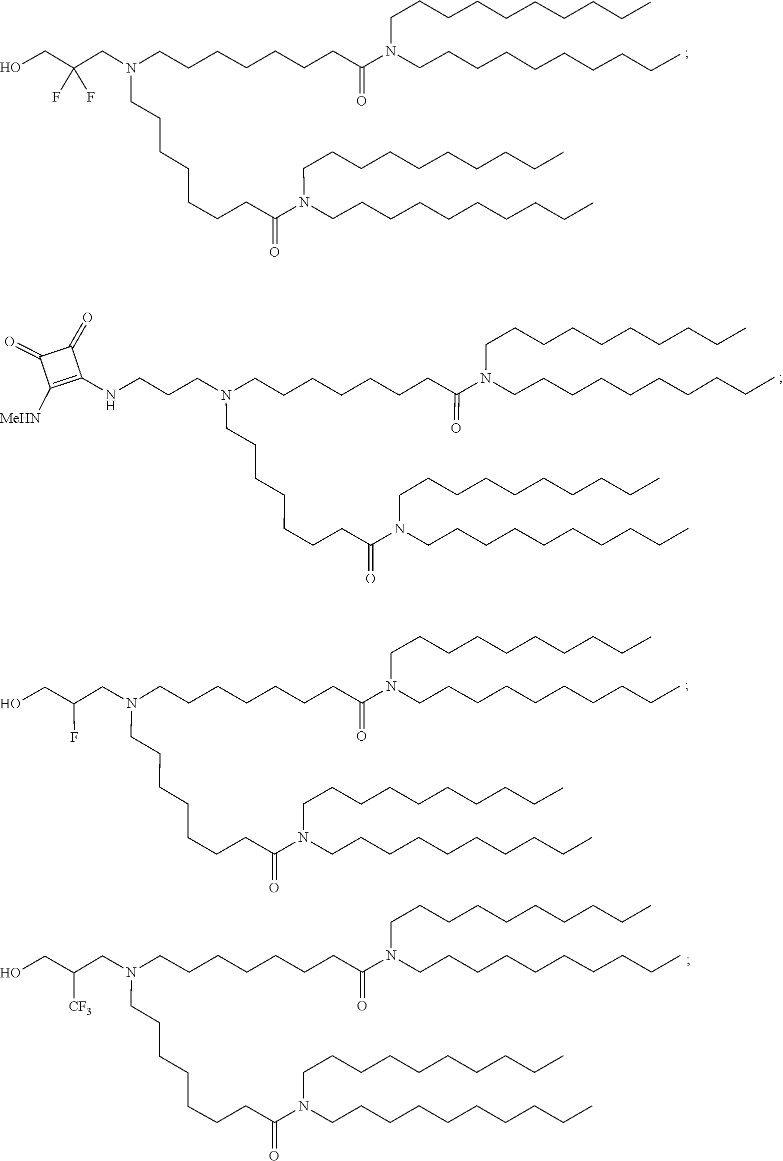 [SMILES: CCCCCCCCCCN(CCCCCCCCCC)C(=O)CCCCCCCN(CCCCCCCC(=O)N(CCCCCCCCCC)CCCCCCCCCC)CC(F)(F)CO ; CCCCCCCCCCN(CCCCCCCCCC)C(=O)CCCCCCCN(CCCCCCCC(=O)N(CCCCCCCCCC)CCCCCCCCCC)CCCNc1c(NC)c(=O)c1=O ; CCCCCCCCCCN(CCCCCCCCCC)C(=O)CCCCCCCN(CCCCCCCC(=O)N(CCCCCCCCCC)CCCCCCCCCC)CC(F)CO ; CCCCCCCCCCN(CCCCCCCCCC)C(=O)CCCCCCCN(CCCCCCCC(=O)N(CCCCCCCCCC)CCCCCCCCCC)CC(CO)C(F)(F)F]

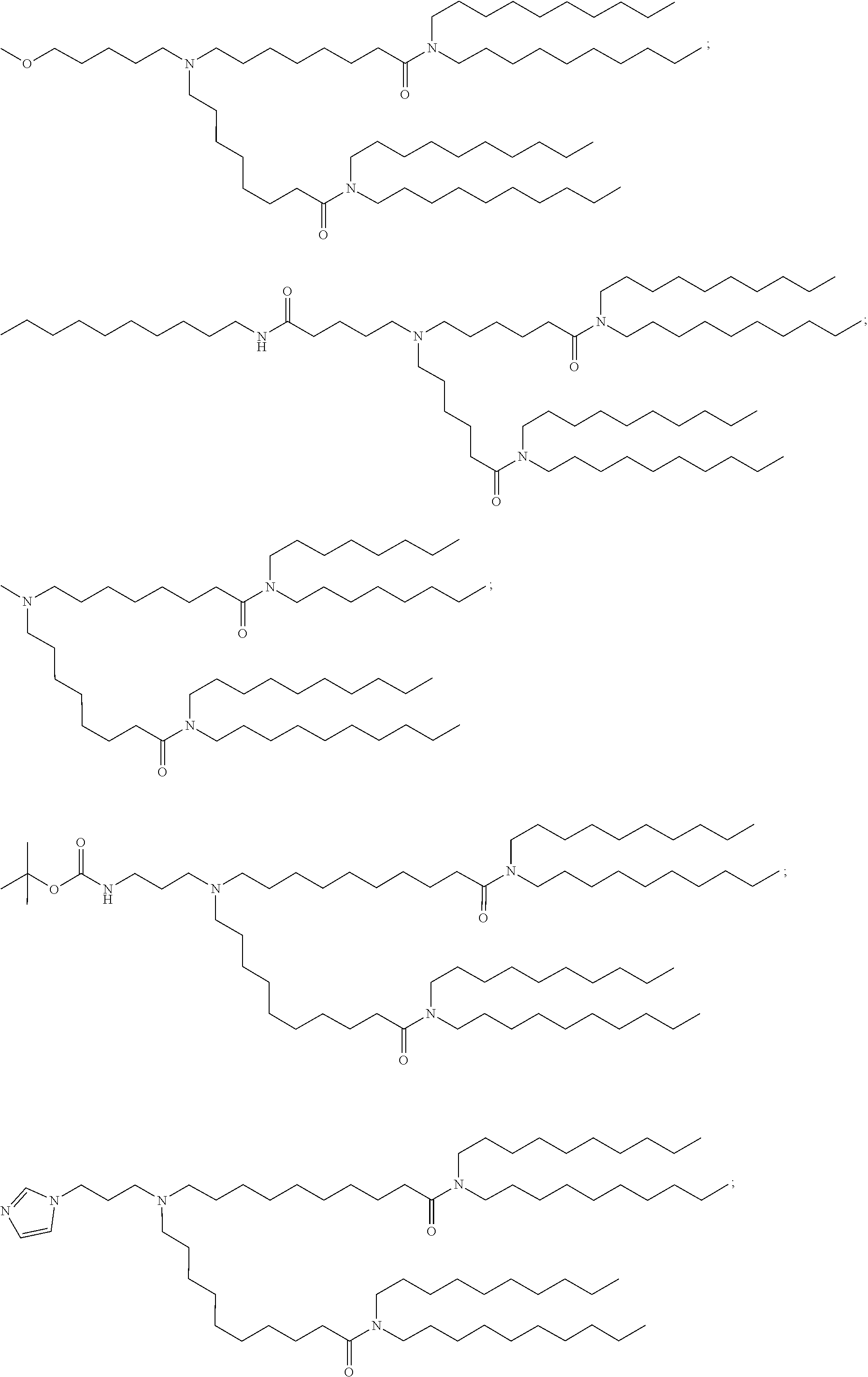

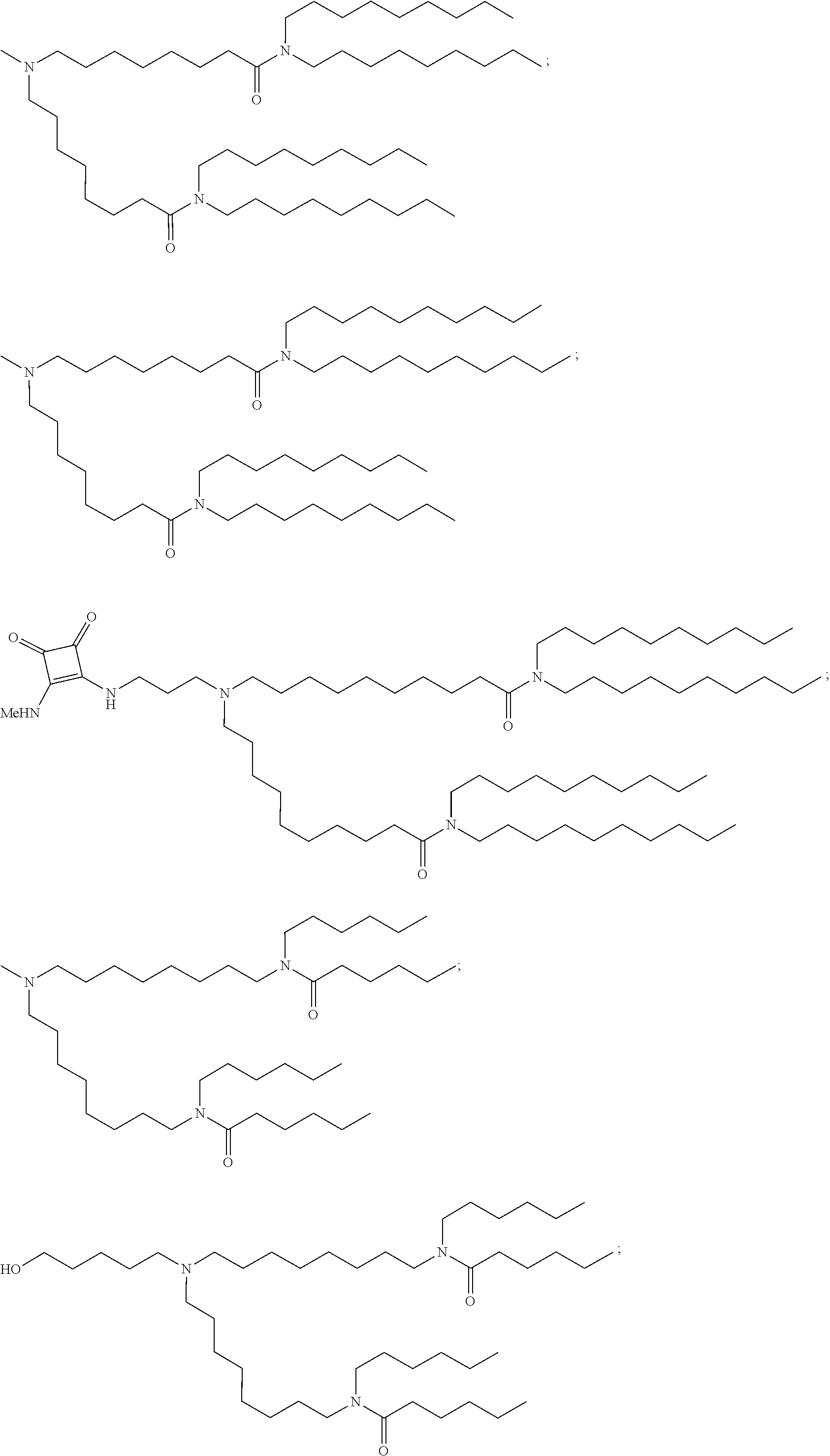

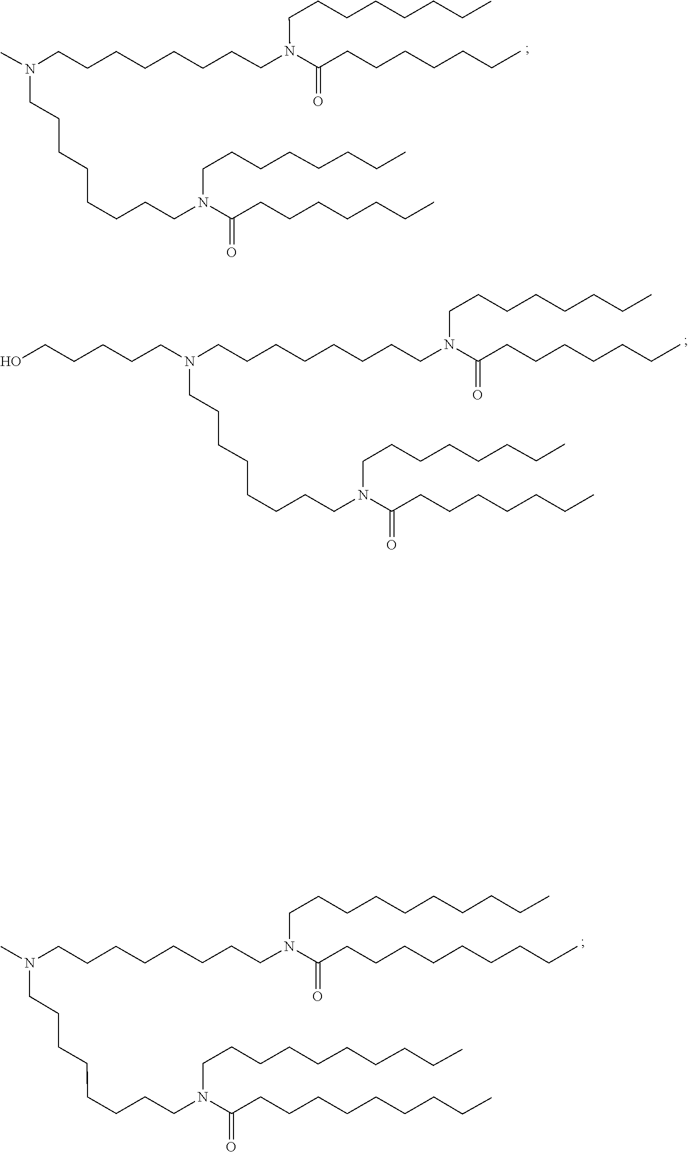

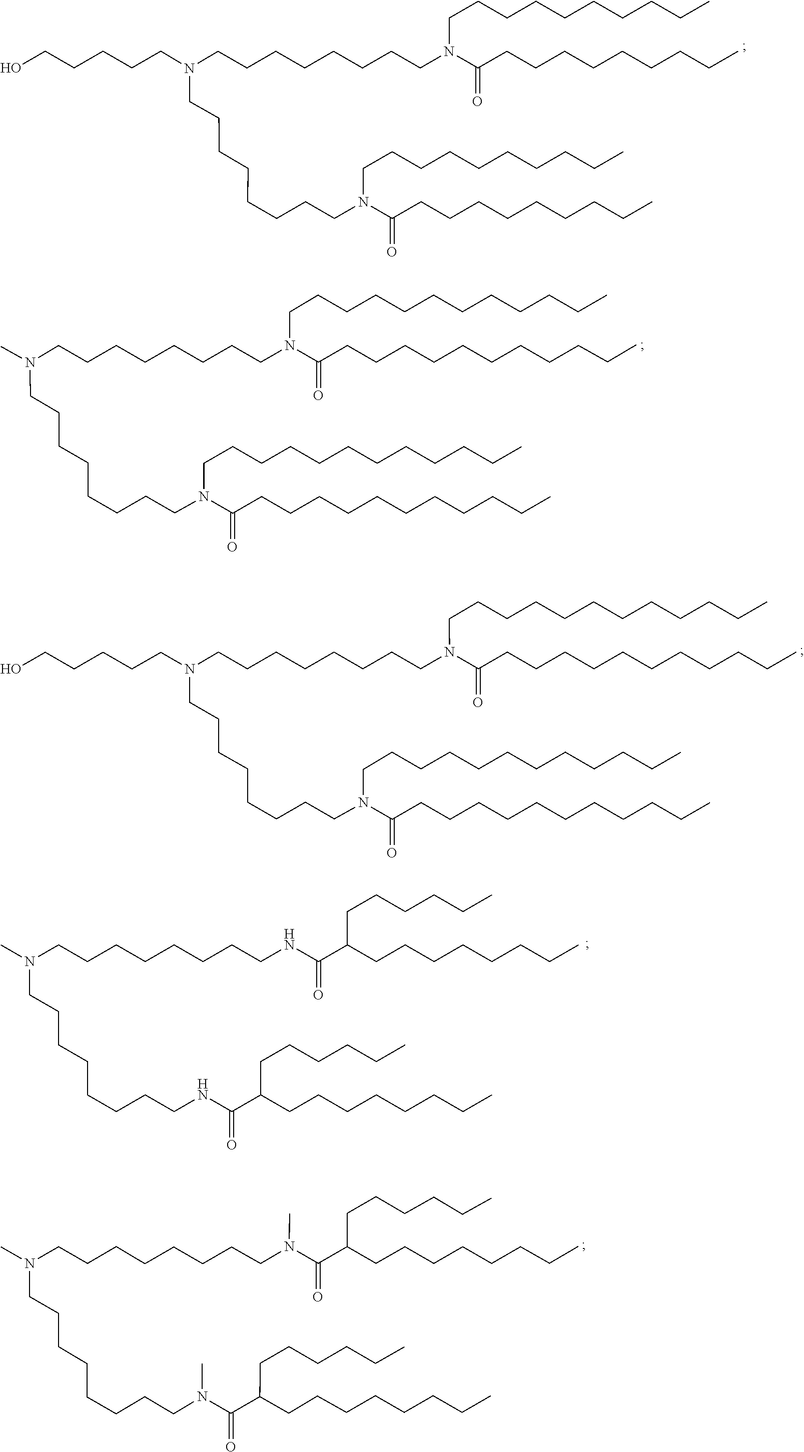

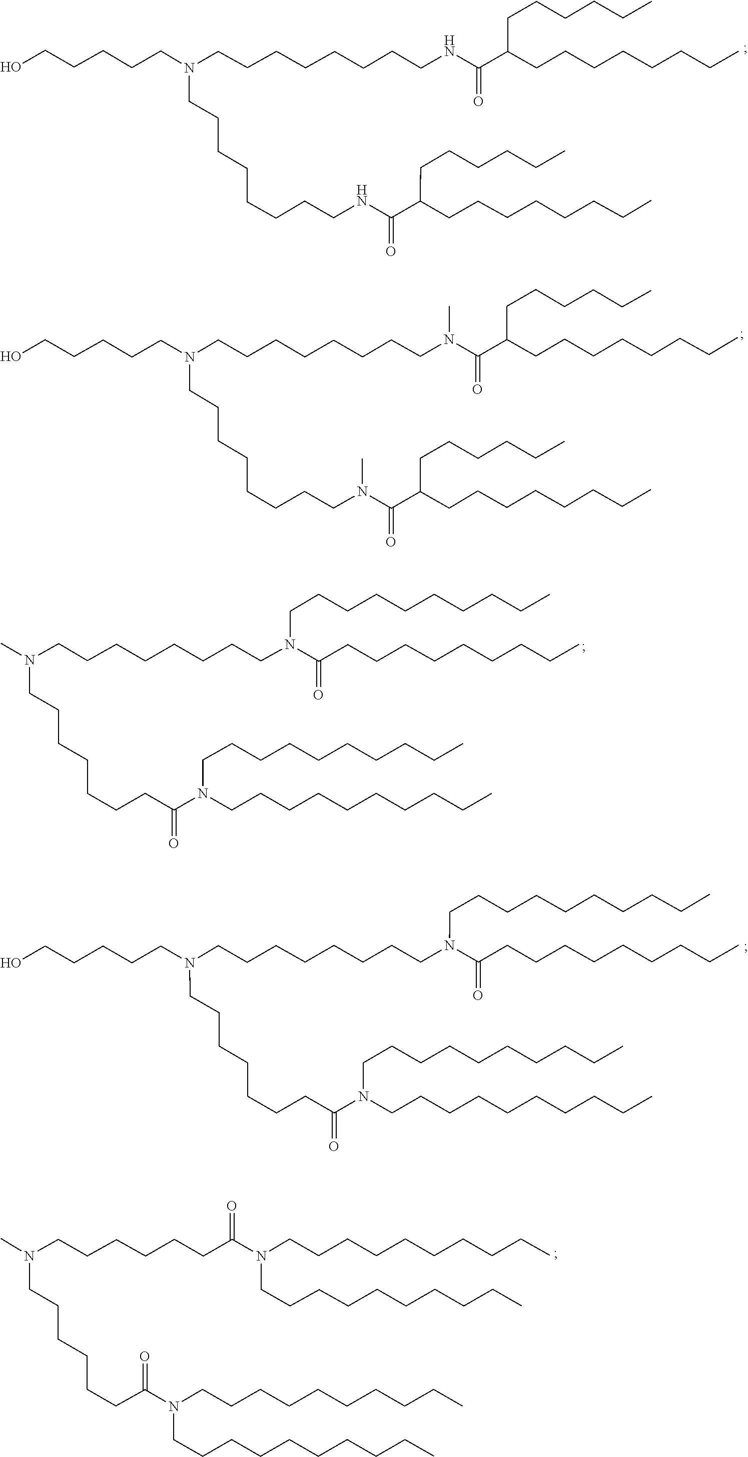

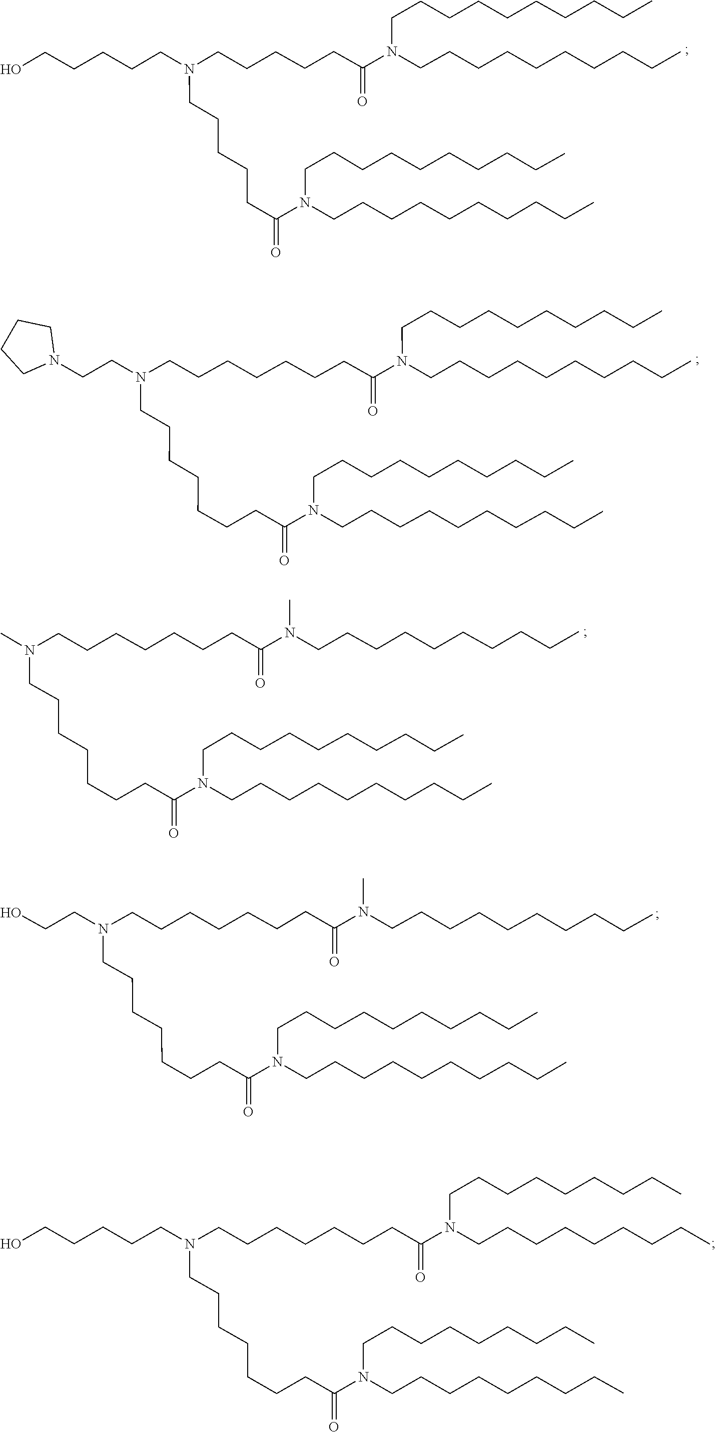

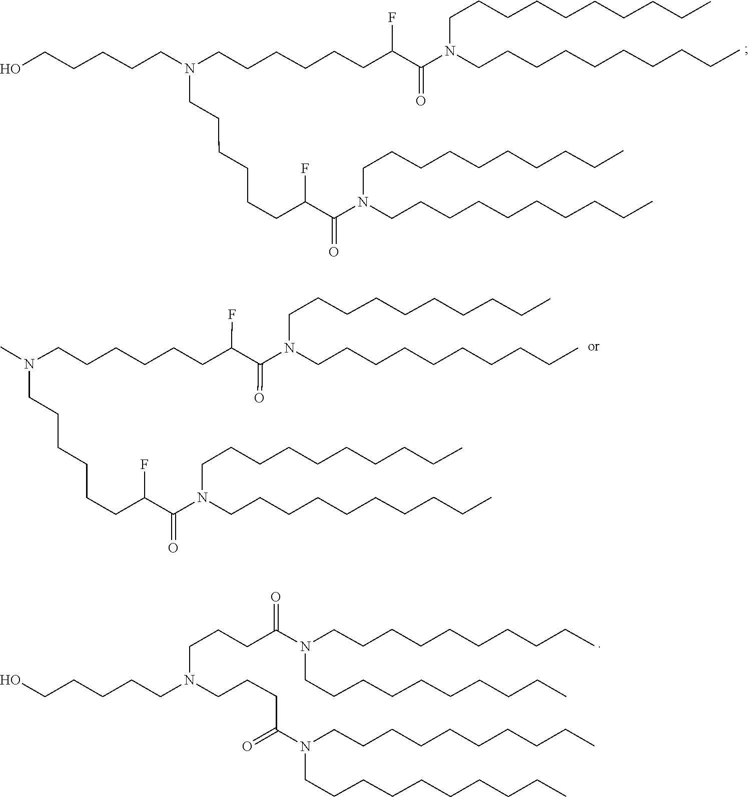

44. A lipid nanoparticle comprising a compound having the following structure (I): 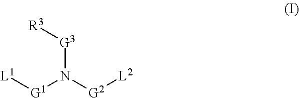
or a pharmaceutically acceptable salt, prodrug or stereoisomer thereof, wherein:
L¹ is —NR^{a}C(═O)R¹ or —C(═O)NR^{b}R^{c}:
L² is —NR^{d}C(═O)R² or —C(═O)NR^{c}R^{f};
G¹ and G² are each independently C₂-C₁₂ alkylene, or C₂-C₁₂ alkenylene;
G³ is C₁-C₂₄ alkylene, or C₂-C₂₄ alkenylene;
R^{a}, R^{b}, R^{d} and R^{e} are each independently H, C₁-C₁₆ alkyl or C₂-C₁₆ alkenyl;
R^{c} and R^{f} are each independently C₁-C₁₆ alkyl or C₂-C₁₆ alkenyl;
R¹ and R² are each independently C₆-C₂₄ alkyl or C₆-C₂₄ alkenyl;
R³ is H, —OH, CN, —N(R⁴)R⁵: —C(═O)N(R⁴)R⁵; —N(R⁴)C(═O)R⁵; —N(R⁴)C(═O)OR; —C(═O)OR⁶, —OC(═O)R⁶, —OR⁷, heteroaryl or aryl;
R⁴ and R⁵ are each independently is H, C₁-C₁₂ alkyl, C₃-C₆ cycloalkyl or C₃-C₆ cycloalkenyl, or R⁴ and R⁵, together with the nitrogen or carbon atom to which they are bound, form a 5 to 7-membered heterocyclic ring;
R⁶ is H, C₁-C₁₂ alkyl, C₂-C₁₂ alkenyl or aralkyl;
R⁷ is C₁-C₁₂ alkyl optionally substituted with hydroxyl or alkoxy; and
wherein each alkyl, alkenyl, alkylene, alkenylene, aryl and aralkyl is independently substituted or unsubstituted unless otherwise specified.

45. A composition comprising the compound of claim 1 and a therapeutic agent.

46-54. (canceled)

55. A method for inducing expression of a protein in a subject, comprising administering to the subject a lipid nanoparticle comprising an mRNA encoding the protein, and wherein the lipid nanoparticle comprises a compound having the following structure (I): 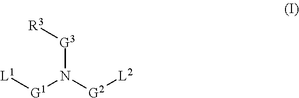
or a pharmaceutically acceptable salt or stereoisomer thereof, wherein:
L¹ is —NR^{a}C(═O)R¹ or —C(═O)NR^{b}R^{c};
L² is —NR^{d}C(═O)R² or —C(═O)NR^{c}R^{f};
G¹ and G² are each independently C₂-C₁₂ alkylene, or C₂-C₁₂ alkenylene;
G³ is C₁-C₂₄ alkylene, or C₂-C₂₄ alkenylene;
R^{a}, R^{b}, R^{d} and R^{e} are each independently H, C₁-C₁₆ alkyl or C₂-C₁₆ alkenyl;
R^{c} and R^{f} are each independently C₁-C₁₆ alkyl or C₂-C₁₆ alkenyl;
R¹ and R² are each independently C₆-C₂₄ alkyl or C₆-C₂₄ alkenyl;
R³ is H, —OH, CN, —N(R⁴)R⁵; —C(═O)N(R⁴)R⁵; —N(R⁴)C(═O)R⁵; —N(R⁴)C(═O)OR⁵; —C(═O)OR⁶, —OC(═O)R⁶, —OR⁷, heteroaryl or aryl;
R⁴ and R⁵ are each independently is H, C₁-C₁₂ alkyl, C₃-C₆ cycloalkyl or C₃-C₆ cycloalkenyl, or R⁴ and R⁵, together with the nitrogen or carbon atom to which they are bound, form a 5 to 7-membered heterocyclic ring;
R⁶ is H, C₁-C₁₂ alkyl, C₂-C₁₂ alkenyl or aralkyl;
R⁷ is C₁-C₁₂ alkyl optionally substituted with hydroxyl or alkoxy; and
wherein each alkyl, alkenyl, alkylene, alkenylene, aryl and aralkyl is independently substituted or unsubstituted unless otherwise specified.
